SEQUENCE LISTING (73 sequences)

SEQ ID NO: 1 - protein, 275 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSR

SEQ ID NO: 2 - protein, 311 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 3 - protein, 19 aa, synthetic construct
  REGION 1..19: note = Synthetic
ATNFSLLKQAGDVEENPGP

SEQ ID NO: 4 - protein, 227 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 5 - protein, 262 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRAD

SEQ ID NO: 6 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 7 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 55 aa, Homo sapiens
KNRKAKAKPVTRGAGAGGRQRGQNKERPPPVPNPDYEPIRKGQRDLYSGLNQRRI

SEQ ID NO: 9 - protein, 612 aa, synthetic construct
  REGION 1..612: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 10 - protein, 883 aa, synthetic construct
  REGION 1..883: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 769 aa, synthetic construct
  REGION 1..769: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSKNRKAKAKPVTRGAGAGGRQRGQNKERPPPVPNPDYEPIRKGQRDLYSGLNQRRIAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKFWVLVVVGGVLACYSLLVTVAFIIFWVRSKRSRLLHSDYMNMTPRRPGPTRKHYQPYAPPRDFAAYRSKNRKAKAKPVTRGAGAGGRQRGQNKERPPPVPNPDYEPIRKGQRDLYSGLNQRRI

SEQ ID NO: 12 - DNA, 1839 bp, synthetic construct
  misc_feature 1..1839: note = Synthetic
  source 1..1839: mol_type = other DNA
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagccgggccaagcggtctgggtctggggccaccaacttcagcctgctgaagcaggccggcgacgtggaggagaaccccggccccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggctatggtcaagagaaaggattccagaggctaa

SEQ ID NO: 13 - DNA, 2652 bp, synthetic construct
  misc_feature 1..2652: note = Synthetic
  source 1..2652: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - DNA, 2310 bp, synthetic construct
  misc_feature 1..2310: note = Synthetic
  source 1..2310: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtaaaaaccgcaaagctaaagctaaacccgtcactaggggggccggagcaggagggcgccagcgcggtcagaataaagaacgccctcctcccgtccctaatcctgattacgaaccgattagaaaggggcaaagagatctctacagcggactcaaccaacggagaattgccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagaatagaaaggccaaggccaagcctgtgacacgaggagcgggtgctggcggcaggcaaaggggacaaaacaaggagaggccaccacctgttcccaacccagactatgagcccatccgcaaaggccagcgggacctgtattctggcctgaatcagagacgcatctaa

SEQ ID NO: 15 - protein, 141 aa, synthetic construct [sequence omitted]

SEQ ID NO: 16 - DNA, 540 bp, synthetic construct
  misc_feature 1..540: note = Synthetic
  source 1..540: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 204 bp, Homo sapiens
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 18 - DNA, 204 bp, synthetic construct
  misc_feature 1..204: note = Synthetic
  source 1..204: mol_type = other DNA
ttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagt

SEQ ID NO: 19 - DNA, 336 bp, Homo sapiens
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 336 bp, synthetic construct
  misc_feature 1..336: note = Synthetic
  source 1..336: mol_type = other DNA
cgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccaga

SEQ ID NO: 21 - DNA, 540 bp, synthetic construct
  misc_feature 1..540: note = Synthetic
  source 1..540: mol_type = other DNA
ttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccaga

SEQ ID NO: 22 - DNA, 165 bp, Homo sapiens
  source 1..165: mol_type = other DNA
aagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatc

SEQ ID NO: 23 - DNA, 165 bp, synthetic construct
  misc_feature 1..165: note = Synthetic
  source 1..165: mol_type = other DNA
aagaatagaaaggccaaggccaagcctgtgacacgaggagcgggtgctggcggcaggcaaaggggacaaaacaaggagaggccaccacctgttcccaacccagactatgagcccatccgcaaaggccagcgggacctgtattctggcctgaatcagagacgcatc

SEQ ID NO: 24 - DNA, 165 bp, synthetic construct
  misc_feature 1..165: note = Synthetic
  source 1..165: mol_type = other DNA
aaaaaccgcaaagctaaagctaaacccgtcactaggggggccggagcaggagggcgccagcgcggtcagaataaagaacgccctcctcccgtccctaatcctgattacgaaccgattagaaaggggcaaagagatctctacagcggactcaaccaacggagaatt

SEQ ID NO: 25 - protein, 9 aa, Homo sapiens
SLLMWITQC

SEQ ID NO: 26 - protein, 40 aa, Homo sapiens
KIEVMYPPPYLDNEKSNGTIIHVKGKHLCPSPLFPGPSKP

SEQ ID NO: 27 - protein, 27 aa, Homo sapiens
FWVLVVVGGVLACYSLLVTVAFIIFWV

SEQ ID NO: 28 - protein, 41 aa, Homo sapiens
RSKRSRLLHSDYMNMTPRRPGPTRKHYQPYAPPRDFAAYRS

SEQ ID NO: 29 - protein, 42 aa, Homo sapiens
KRGRKKLLYIFKQPFMRPVQTTQEEDGCSCRFPEEEEGGCEL

SEQ ID NO: 30 - protein, 46 aa, Homo sapiens
AKPTTTPAPRPPTPAPTIASQPLSLRPEACRPAAGGAVHTRGLDFA

SEQ ID NO: 31 - protein, 26 aa, Homo sapiens
CDIYIWAPLAGTCGVLLLSLVITLYC

SEQ ID NO: 32 - protein, 883 aa, synthetic construct
  REGION 1..883: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKCDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 885 aa, synthetic construct
  REGION 1..885: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 967 aa, synthetic construct
  REGION 1..967: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 883 aa, synthetic construct
  REGION 1..883: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 771 aa, synthetic construct
  REGION 1..771: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCEL

SEQ ID NO: 37 - protein, 884 aa, synthetic construct
  REGION 1..884: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 882 aa, synthetic construct
  REGION 1..882: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKCDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 965 aa, synthetic construct
  REGION 1..965: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKCDIYIWAPLAGTCGVLLLSLVITLYCRSKRSRLLHSDYMNMTPRRPGPTRKHYQPYAPPRDFAAYRSRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCELKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 1059 aa, synthetic construct
  REGION 1..1059: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 859 aa, synthetic construct
  REGION 1..859: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADGSPKAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSGSPKCDIYIWAPLAGTCGVLLLSLVITLYCKRGRKKLLYIFKQPFMRPVQTTQEEDGCSCRFPEEEEGGCEL

SEQ ID NO: 42 - DNA, 120 bp, Homo sapiens
  source 1..120: mol_type = other DNA
aaaattgaagttatgtatcctcctccttacctagacaatgagaagagcaatggaaccattatccatgtgaaagggaaacacctttgtccaagtcccctatttcccggaccttctaagccc

SEQ ID NO: 43 - DNA, 120 bp, Homo sapiens
  source 1..120: mol_type = other DNA
aagatcgaggtaatgtacccaccgccctatcttgataacgaaaaatctaacggtacaataattcacgtcaagggcaagcatttgtgcccttccccgttgttcccgggcccaagcaaaccg

SEQ ID NO: 44 - DNA, 81 bp, Homo sapiens
  source 1..81: mol_type = other DNA
ttttgggtgctggtggtggttggtggagtcctggcttgctatagcttgctagtaacagtggcctttattattttctgggtg

SEQ ID NO: 45 - DNA, 81 bp, Homo sapiens
  source 1..81: mol_type = other DNA
ttctgggttctcgtcgtcgtgggaggtgtgttagcatgttactctctcttggttactgtcgctttcataatcttttgggtc

SEQ ID NO: 46 - DNA, 123 bp, Homo sapiens
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 47 - DNA, 123 bp, synthetic construct
  misc_feature 1..123: note = Synthetic
  source 1..123: mol_type = other DNA
cgctcaaaacgctctcgcttgttacattccgattatatgaatatgacacctaggagacctggcccgactaggaaacactatcaaccttacgcacctcccagagattttgctgcttacaggagt

SEQ ID NO: 48 - DNA, 126 bp, Homo sapiens
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 49 - DNA, 126 bp, synthetic construct
  misc_feature 1..126: note = Synthetic
  source 1..126: mol_type = other DNA
aagagagggcgtaaaaagctgctctacatctttaagcagcctttcatgcgtcctgttcagacaacacaggaagaggacggatgctcttgcaggttccctgaggaggaggagggtggttgcgagctc

SEQ ID NO: 50 - DNA, 138 bp, Homo sapiens
  source 1..138: mol_type = other DNA
gctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcc

SEQ ID NO: 51 - DNA, 138 bp, synthetic construct
  misc_feature 1..138: note = Synthetic
  source 1..138: mol_type = other DNA
gctaagcccactactaccccagctcccaggcctcccacacctgccccaacaatcgccagccagccactgtcccttaggcccgaggcctgtaggcccgccgccggaggagccgtgcacacccgcggactggattttgct

SEQ ID NO: 52 - DNA, 78 bp, Homo sapiens
  source 1..78: mol_type = other DNA
tgtgatatctacatctgggcgcccttggccgggacttgtggggtccttctcctgtcactggttatcaccctttactgc

SEQ ID NO: 53 - DNA, 78 bp, synthetic construct
  misc_feature 1..78: note = Synthetic
  source 1..78: mol_type = other DNA
tgcgacatttatatttgggcccctctcgctggcacatgcggcgtgttgttgctcagcctcgtgattacactttattgt

SEQ ID NO: 54 - DNA, 2652 bp, synthetic construct
  misc_feature 1..2652: note = Synthetic
  source 1..2652: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagtgcgacatttatatttgggcccctctcgctggcacatgcggcgtgttgttgctcagcctcgtgattacactttattgtaagagagggcgtaaaaagctgctctacatctttaagcagcctttcatgcgtcctgttcagacaacacaggaagaggacggatgctcttgcaggttccctgaggaggaggagggtggttgcgagctccgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 55 - DNA, 2658 bp, synthetic construct
  misc_feature 1..2658: note = Synthetic
  source 1..2658: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtcaagagagggcgtaaaaagctgctctacatctttaagcagcctttcatgcgtcctgttcagacaacacaggaagaggacggatgctcttgcaggttccctgaggaggaggagggtggttgcgagctccgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaattttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 56 - DNA, 2904 bp, synthetic construct
  misc_feature 1..2904: note = Synthetic
  source 1..2904: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtaagagagggcgtaaaaagctgctctacatctttaagcagcctttcatgcgtcctgttcagacaacacaggaagaggacggatgctcttgcaggttccctgaggaggaggagggtggttgcgagctccgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 57 - DNA, 2652 bp, synthetic construct
  misc_feature 1..2652: note = Synthetic
  source 1..2652: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 58 - DNA, 2316 bp, synthetic construct
  misc_feature 1..2316: note = Synthetic
  source 1..2316: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaa

SEQ ID NO: 59 - DNA, 2655 bp, synthetic construct
  misc_feature 1..2655: note = Synthetic
  source 1..2655: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaattttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 60 - DNA, 2649 bp, synthetic construct
  misc_feature 1..2649: note = Synthetic
  source 1..2649: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagtgcgacatttatatttgggcccctctcgctggcacatgcggcgtgttgttgctcagcctcgtgattacactttattgtcgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 61 - DNA, 2898 bp, synthetic construct
  misc_feature 1..2898: note = Synthetic
  source 1..2898: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaagtgcgacatttatatttgggcccctctcgctggcacatgcggcgtgttgttgctcagcctcgtgattacactttattgtaggagtaagaggagcaggctcctgcacagtgactacatgaacatgactccccgccgccccgggcccacccgcaagcattaccagccctatgccccaccacgcgacttcgcagcctatcgctcccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaagagagggcgtaaaaagctgctctacatctttaagcagcctttcatgcgtcctgttcagacaacacaggaagaggacggatgctcttgcaggttccctgaggaggaggagggtggttgcgagctcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 62 - DNA, 3180 bp, synthetic construct
  misc_feature 1..3180: note = Synthetic
  source 1..3180: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaaggcaaaaccgacgaccacccctgcccccaggcctcctactcccgccccgacgattgccagccaaccgttaagtttaagaccggaagcatgtagaccggcagctggtggggctgttcatacacgtggcttagattttgcgcctaggaagatcgaggtaatgtacccaccgccctatcttgataacgaaaaatctaacggtacaataattcacgtcaagggcaagcatttgtgcccttccccgttgttcccgggcccaagcaaaccgttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaagctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 63 - DNA, 2580 bp, synthetic construct
  misc_feature 1..2580: note = Synthetic
  source 1..2580: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggctctcctaaggcaaaaccgacgaccacccctgcccccaggcctcctactcccgccccgacgattgccagccaaccgttaagtttaagaccggaagcatgtagaccggcagctggtggggctgttcatacacgtggcttagattttgcgcctaggaagatcgaggtaatgtacccaccgccctatcttgataacgaaaaatctaacggtacaataattcacgtcaagggcaagcatttgtgcccttccccgttgttcccgggcccaagcaaaccgttctgggttctcgtcgtcgtgggaggtgtgttagcatgttactctctcttggttactgtcgctttcataatcttttgggtccgctcaaaacgctctcgcttgttacattccgattatatgaatatgacacctaggagacctggcccgactaggaaacactatcaaccttacgcacctcccagagattttgctgcttacaggagtcgggtcaaattttcacgctccgctgatgctcctgcctatcaacaagggcaaaatcaattgtacaatgaattgaacttgggtagaagggaagaatatgacgtgctcgataaacggagggggagagatccagaaatgggcggtaaaccacggcgcaaaaatccacaagagggattgtataacgagctccaaaaggacaaaatggcagaagcttattcagaaataggaatgaagggggaaaggagacgaggtaaaggtcatgacggattgtatcaaggattgtcaaccgctactaaagatacatatgatgctttgcatatgcaagctttgcctcccagagccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccggctccccaaaatgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaa

SEQ ID NO: 64 - protein, 612 aa, synthetic construct
  REGION 1..612: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRGAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSR

SEQ ID NO: 65 - DNA, 1839 bp, synthetic construct
  misc_feature 1..1839: note = Synthetic
  source 1..1839: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggctatggtcaagagaaaggattccagaggcgccaagcggtctgggtctggggccaccaacttcagcctgctgaagcaggccggcgacgtggaggagaaccccggccccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagccggtaa

SEQ ID NO: 66 - protein, 48 aa, synthetic construct
  REGION 1..48: note = Synthetic
CDVKLVEKSFETDTNLNFQNLSVIGFRILLLKVAGFNLLMTLRLWSSR

SEQ ID NO: 67 - protein, 7 aa, synthetic construct
  REGION 1..7: note = Synthetic
AKRSGSG

SEQ ID NO: 68 - protein, 49 aa, synthetic construct
  REGION 1..49: note = Synthetic
CGFTSESYQQGVLSATILYEILLGKATLYAVLVSALVLMAMVKRKDSRG

SEQ ID NO: 69 - protein, 4 aa, synthetic construct
  REGION 1..4: note = Synthetic
GSPK

SEQ ID NO: 70-71 - skipped (2 entries)

SEQ ID NO: 72 - DNA, 14 bp, synthetic construct
  misc_feature 1..14: note = Synthetic
  source 1..14: mol_type = other DNA
cagccagcggccgc

SEQ ID NO: 73 - skipped

(19) United States
(12) Patent Application Publication
(10) Pub. No.: US 2026/0116949 A1
(43) Pub. Date: Apr. 30, 2026

(54) COMPOSITION AND METHOD FOR ADOPTIVE IMMUNOTHERAPY

(71) Applicant: Wen Yang, Sharon, MA (US)

(72) Inventor: Wen Yang, Sharon, MA (US)

(21) Appl. No.: 19/428,820

(22) Filed: Dec. 22, 2025

Related U.S. Application Data
(62) Division of application No. 17/628,032, now Pat. No. 12,534,506.
(60) Provisional application No. 62/877,331, filed on Jul. 23, 2019.
(60) Provisional application No. 63/044,059, filed on Jun. 25, 2020.

(51) Int. Cl. C07K 14/725; A61K 40/11; A61K 40/31; A61K 40/32; A61K 40/42; C07K 14/705; C07K 16/30; C12N 5/0783

(52) U.S. Cl. CPC C07K 14/7051; A61K 40/11; A61K 40/31; A61K 40/32; A61K 40/4255; A61K 40/4269; C07K 14/70521; C07K 14/70578; C07K 16/30; C12N 5/0636; A61K 2239/31; A61K 2239/38; A61K 2239/46; C07K 2319/02; C07K 2319/03; C07K 2319/33

(57) ABSTRACT

The present invention relates to agents, compositions, and methods to confer and/or increase immune responses mediated by cellular immunotherapy.

### CROSS REFERENCE TO RELATED APPLICATION

This application is a divisional application of U.S. patent application Ser. No. 17/628,032, filed on Jan. 18, 2022, which is a U.S. national phase of International Patent Application No. PCT/US20/43072, filed on Jul. 22, 2020, which claims priority to U.S. Provisional Application No. 62/877,331 filed on Jul. 23, 2019 and U.S. Provisional Application No. 63/044,059 filed on Jun. 25, 2020. The contents of the applications are incorporated herein by reference in their entireties.

### REFERENCE TO AN ELECTRONIC SEQUENCE LISTING

The contents of the electronic sequence listing (180423.00212SeqList.xml; Size: 131,857 bytes; and Date of Creation: Dec. 19, 2025) is herein incorporated by reference in its entirety.

### FIELD OF THE INVENTION

This invention relates to recombinant antigen receptors and uses thereof. T cells engineered to express such antigen receptors are useful in the treatment of diseases characterized by expression of one or more antigens bound by the antigen receptors.

### BACKGROUND

Adoptive immunotherapy involves administration of immune effector cells into a patient to produce therapeutic effects. The advent of chimeric antigen receptor (CAR) T cells has provided a useful tool to improve adoptive immunotherapy. To that end, use of genetic engineering to insert antigen-targeted receptors of defined specificity into T cells has greatly extended the capabilities of adoptive immunotherapy. CARs are a type of antigen-targeted receptor composed of intracellular T cell signaling domains fused to extracellular antigen-binding domains. Antibody-based CARs directly recognize cell surface antigens, independent of major histocompatibility complex (MHC)-mediated presentation, permitting the use of a single receptor construct specific for any given antigen in all patients. Initial CARs fused antigen-recognition domains to the CD3ζ (CD3zeta) activation chain of the T cell receptor (TCR) complex. Subsequent CAR iterations have included secondary costimulatory signals in tandem with CD3ζ including intracellular domains from CD28 or a variety of TNF receptor family molecules such as 4-1BB (CD137) and OX40 (CD134). Third generation receptors include two different costimulatory signals in addition to CD3ζ most commonly from CD28 and 4-1BB. The second and third generation of antibody-based CARs improved antitumor efficacy in vitro and in vivo.

Transgenic T cell receptors (tTCRs) and TCR-based CARs (TCR-CARs) are different from antibody-based CARs, primarily in which tTCR and TCR-based CARs bind to the antigen in the MHC (HLA) restricted fashion. Thus, the tTCRs and TCR-based CARs greatly expand the list of possible targets. Creation of TCR-CAR of 2^{nd} gen that comprises the cytoplasmic domains of human CD28 and CD3zeta (CD3Z) or cytoplasmic domains of human CD28 and CD3epsilon (CD3E) in two separate viral vector format were described previously (Govers et al., Journal of Immunology, 2014, 193:5315-5326), and that comprises the cytoplasmic domains of human CD28 and CD3zeta in a single viral vector form was previously described in the U.S. Pat. No. 9,206,440 and Im E J et al., Recombination-deletion between homologous cassettes in retrovirus is suppressed via a strategy of degenerate codon substitution. Molecular Therapy—Methods & Clinical Development (2014) Article number: 14022). In addition, creation of TCR-CAR of 2^{nd} gen that comprises a single chain TCR and the cytoplasmic domains of human CD28 and CD3zeta in a single viral vector format were also described previously (Walseng et al., A TCR-based Chimeric Antigen Receptor. Sci Rep. 2017 Sep. 6; 7 (1): 10713). Third generation receptors include two different costimulatory signals in addition to CD3ζ, most commonly from CD28 and 4-1BB. The the second and third generation of antibody-based CARs improved antitumor efficacy in vitro and in vivo.

Unlike to 2^{nd} or 3^{rd} generation of antibody-based CARs, tTCR and TCR-based CARs face various obstacles, such as loss of stability for efficient tTCR or TCR-based CAR expression and activity. While tTCR itself lacks co-stimulatory signaling in its own molecule, current reported TCR-based CARs either lack sufficient co-stimulatory signaling elements such as 4-1BB or lacks optimal designs for optimal cell surface expression and CAR-mediated T cell activity. There is a need for novel TCR-based CAR designs and adoptive therapies that provide cells with enhanced functionalities.

### SUMMARY

This disclosure addresses the need mentioned above in a number of aspects. In one aspect, the disclosure provides an antigen receptor comprising (I) a first polypeptide chain that comprises an extracellular domain comprising a TCR beta chain or an antigen-binding fragment thereof, a transmembrane (TM or Tm) domain (TMD), and a cytoplasmic domain (Cyt or cyt); and (II) a second polypeptide chain that comprises an extracellular domain (Ec) comprising a TCR alpha chain or an antigen-binding fragment thereof, a transmembrane domain, and a cytoplasmic domain. The TCR beta chain and the TCR alpha chain form an antigen-binding site. In embodiments, the first polypeptide chain is substantially different from the second polypeptide chain in one or more of the transmembrane domain, and the cytoplasmic domain.

The cytoplasmic domain of the first polypeptide chain or of the second polypeptide chain comprises (a) 0, 1, or 2 copies of a cytoplasmic domain of a human 4-1BB or a fragment thereof, or (b) 0, 1, or 2 copies of a cytoplasmic domains of human CD3zeta (CD3Z) or a fragment thereof, or (c) 0, 1, or 2 copies of a cytoplasmic domains of human CD3 epsilon (CD3E) or a fragment thereof, or (d) 0, 1, or 2 copies of a cytoplasmic domain of human CD28 or a fragment thereof.

In the antigen receptor, the transmembrane domain of the first or the second polypeptide chain may comprise one selected from the group consisting of a transmembrane domain of CD8 and a transmembrane domain of CD28.

In some embodiments, the antigen receptor comprises 1 or 2 copies of the cytoplasmic domain of a human 4-1BB. Examples of such antigen receptor include those encoded by vectors NT 4, 5, 6, 21, 22, 23, 24 25, and 27 described herein. In some examples, the antigen receptor can comprise 1 or 2 copies of the cytoplasmic domain of human CD3zeta. In other examples, the antigen receptor may comprise 1 or 2 copies of the cytoplasmic domain of human CD28. Examples include those encoded by vectors NT 6, 21, 22, 23, 24 25, and 27.

In one embodiment, the antigen receptor comprises only 1 copy of the cytoplasmic domain of human 4-1BB, 1 copy of the cytoplasmic domains of human CD28, and 1 copy of the cytoplasmic domains of human CD3zeta. The antigen receptor can further comprise only one copy of the transmembrane domain of CD28 and one copy of the transmembrane domain of CD8. In one example (such as that encoded by NT22), the first polypeptide chain may contain the extracellular domain of a TCR beta chain, 1 copy of the CD28 transmembrane domain, 1 copy of the cytoplasmic domains of human CD28, and 1 copy of the cytoplasmic domains of human CD3zeta, while the second polypeptide chain may contain the extracellular domain of a TCR alpha chain, 1 copy of the CD8 transmembrane domain, and 1 copy of the cytoplasmic domain of human 4-1BB.

In one embodiment, the antigen receptor comprises 2 copies of the cytoplasmic domain of human 4-1BB, 2 copies of the cytoplasmic domain of human CD28, and 2 copies of the cytoplasmic domains of human CD3zeta. For example, each of the two chains can contain 1 copy for each of the cytoplasmic domain of human 4-1BB, the cytoplasmic domain of human CD28, and the cytoplasmic domains of human CD3zeta (e.g., that encoded by NT6). Alternatively, one polypeptide chain (e.g., the first polypeptide chain) may contain 2 copies of the cytoplasmic domain of human CD28 while the other (e.g., the second polypeptide chain) may contain 2 copies of the cytoplasmic domain of human 4-1BB. Examples include that encoded by vectors NT25.

In one embodiment of the antigen receptor, one of the two chains has the cytoplasmic domain of human CD3zeta. For instance, the first polypeptide chain may comprise 1 copy of the transmembrane domain of human CD28, 1 copy of the cytoplasmic domain human CD28, and 1 copy of the cytoplasmic domain of human CD3zeta. The second polypeptide chain may comprise 1 copy of the transmembrane domain of human CD8 and 1 copy of the cytoplasmic domain human 4-1BB, but no cytoplasmic domain of human CD3zeta. Examples include that encoded by NT22.

In one embodiment, the first and the second polypeptide chains have different co-stimulatory domains. For instance, the first polypeptide chain may comprise 1 copy of the transmembrane domain of human CD8, 1 copy of the cytoplasmic domain human CD28, and 1 copy of the cytoplasmic domain of human CD3zeta. The second polypeptide chain may comprise 1 copy of the transmembrane domain of human CD8, 1 copy of the cytoplasmic domain human 4-1BB, and 1 copy of the cytoplasmic domain of human CD3zeta. Examples include that encoded by NT24.

In one embodiment, the first polypeptide chain comprises 1 copy of the transmembrane domain of human CD8, 2 copies of the cytoplasmic domain human CD28, and 1 copy of the cytoplasmic domain of human CD3zeta. The second polypeptide chain comprises 1 copy of the transmembrane domain of human CD8, 2 copies of the cytoplasmic domain human 4-1BB, and 1 copy of the cytoplasmic domain of human CD3zeta. Examples include that encoded by NT25.

It was surprising and un-expected that (1) the anti-NY-ESO-1/A2 TCR-based CARs of 2^{nd} gen (e.g., NT2, NT3, NT4, and NT5) and 3^{rd} gen (e.g., NT24) show higher surface expression on virus transduced human T cells as determined by flow cytometry measurement, compared to the TCRs of native form (e.g., NT1 and NT1b), (2) human T cells expressing the anti-NY-ESO-1/A2 TCR-based CARs of 2^{nd} gen (for example Nt2, NT3, NT4, NT5) and 3^{rd} gen (e.g., NT24), are more potent in TCR-CAR-mediated cytokine secretion (e.g., IL-2) and cell expansion post engagement with tumor target cells, compared to those expressing the TCRs of native form (e.g., NT1, NT1b), and (3) in the in vivo therapeutic assay involving a Xenograft Saos-2 tumor murine model, the data suggest that the 2^{nd} gen anti-NY-ESO-1 TCR-CARs (e.g., NT2, NT4) show superior anti-tumor activity than the native from NT1 (NT1a), while the 3^{rd} gen anti-NY-ESO-1/A2 TCR-CAR (e.g., NT24) is significantly more potent that its counter parts of 2^{nd} gen (e.g., NT2, NT4) with only TCR signal 2 only from human CD28 (e.g., NT2) or only from 4-1BB (e.g., NT4), and even more potent than the native form NT1 (e.g., NT1a).

In the antigen receptor described above, the antigen-binding site may bind to a tumor antigen, or a tumor-related antigen (TAA), or a viral antigen in the context of MHC (HLA) restricted fashion. That is, the extracellular domains, when expressed on a cell, bind to the tumor antigen, TAA, or viral antigen, in the context of MHC (HLA) restricted fashion. Various such antigens are known in the art and some examples are listed herein, including the tumor antigen is NY-ESO-1.

Methods of making the antigen receptor and cells expressing it are disclosed herein. The first polypeptide chain and second polypeptide chain can be expressed from two separate expression cassettes or vectors of from one common expression cassette/vector with the aid of an internal ribosomal entry site (IRES).

The two chains can be expressed as one fusion protein. Accordingly, the disclosure also provides a fusion protein comprising the first polypeptide chain and the second polypeptide chain. In that case, the first polypeptide chain and second polypeptide chain may be linked by a protein linker sequence or a self-cleaving peptide sequence. Examples of the self-cleaving peptide sequence include a P2A, E2A, F2A or T2A sequence.

This disclosure also provides an isolated nucleic acid or a set of isolated nucleic acids encoding the antigen receptor or the fusion protein described above. In one embodiment, the disclosure further provides an isolated nucleic acid or a set of isolated nucleic acids may comprise (I) a first nucleic acid sequence encoding the first polypeptide chain containing a first polypeptide segment of at least 10 (e.g., 10, 20, 30, 40, 50, 60, 70, 80, 100, 150, 200, 250, and 300) amino acids (aa.) in length, and (II) a second nucleic acid sequence encoding the second polypeptide chain containing a second polypeptide segment of at least 10 (e.g., 10, 20, 30, 40, 50, 60, 70, 80, 100, 150, 200, 250, and 300) amino acids in length. The first polypeptide segment is at least 90% (e.g., 95%, 96%, 97%, 98%, 99%, or 100%) identical to the second polypeptide segment, and the first nucleic acid sequence and the second nucleic acid sequence contain at least one non-identical codon within the codons that encode identical amino acid residues in the first polypeptide segment and the second polypeptide segment. The first polypeptide chain and the second polypeptide chain comprise an identical polypeptide sequence of greater than 10 amino acids in length; and the first nucleic acid sequence and the second nucleic acid sequence contain at least one non-identical codon within the codons that encode the identical polypeptide sequence of the first and second nucleic acid sequences. In some embodiments, at least 2% (e.g., 5%, 10%, 20%, 30%, 40%, 50%, 60%, 70%, 80%, 90%, 95%, or 100%) of the codons are non-identical. The first nucleic acid sequence and the second nucleic acid sequence are less than 98% (e.g., 95%, 90%, 80%, 70%, 60%, 50%, 40%, 30%, 20%, 10%, 5%, or 0%) identical within the codons.

In some embodiments, the first nucleic acid sequence and the second nucleic acid sequence contain at least one non-identical codon within the codons that encode one or more selected from the group consisting of: (a) the TCR beta chain or an antigen-binding fragment thereof, (b) the TCR alpha chain or an antigen-binding fragment thereof, (c) the transmembrane domain of CD8, (d) the transmembrane domain of CD28, (e) the cytoplasmic domain of CD3Z or a fragment thereof, (f) the cytoplasmic domain of CD3E or a fragment thereof, (g) the cytoplasmic domain of CD28 or a fragment thereof, and (h) the cytoplasmic domain of 4-1BB or a fragment thereof. The isolated nucleic acid may encode one selected from the group consisting of SEQ ID NOs: 9, 64, 10, 11, and 32-41. The isolated nucleic acid or nucleic acids may comprise a sequence selected from the group consisting of SEQ ID NOs: 12, 65, 13, 14, and 54-63.

The above-described nucleic acid or nucleic acids can be used to express the antigen receptors described here. Accordingly, the disclosure further provides a vector comprising the isolated nucleic acid or nucleic acids described above. In some embodiments, the vector is an expression vector, including a viral vector as described herein. When expressing of the first and second polypeptide chains in cells, bicistronic or multicistronic expression vectors can be used. Among various strategies employed to construct bicistronic or multicistronic vectors, an IRES has been widely used. A self-cleaving 2A peptide could also be a good candidate to be used alone or together with an IRES. Accordingly, the first nucleic acid sequence and the second nucleic acid sequence can be linked via a nucleic acid sequence comprising an IRES. The disclosure further provides a cell comprising the above-described antigen receptor, fusion protein, isolated nucleic acid or nucleic acids, or vector. Examples of the cells are described below, including lymphocytes such as T cell.

Also provided is a pharmaceutical composition comprising (i) the above-described nucleic acid/nucleic acids, vector, or cell and (ii) a pharmaceutically acceptable carrier. Such a pharmaceutical composition can be used in a method of treating a tumor, or a viral infection disease. The method comprises administering to a subject in need thereof an effective amount of the pharmaceutical composition.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A and 1B are a set of diagrams showing exemplary antigen receptors. (A) Schematic presentation of four anti-NY-ESO-1 TCR or TCR-based CAR constructs: 1 (NT1); 2 (NT1b); 3 (NT2); and 4 (NT3). (B) Schematic presentation of additional eleven anti-NY-ESO-1 TCR-based CAR constructs: 5 (NT4); 6 (NT5); 7 (NT6); 8 (NT21); 9 (NT22); 10 (NT23); 11 (NT24); 12 (NT25); 13 (NT26); 14 (NT27); and 15 (NT28).

FIGS. 2A, 2B, 2C, and 2D are schematic presentations of four nucleotide sequences encoding anti-NY-ESO-1 TCR or TCR-based CAR constructs. A: NT1 (SEQ ID NO: 12); B: NT1b (SEQ ID NO: 65); C: NT2 (SEQ ID NO: 13); D: NT3 (SEQ ID NO: 14).

FIG. 3A displays the amino acid sequence of anti-NY-ESO-1 TCR alpha chain-2A-TCR beta chain (NT1; aNY-TCRa/b) (SEQ ID NO: 9).

FIG. 3B displays the amino acid sequence of anti-NY-ESO-1 TCR beta chain-2A-TCR alpha chain (NT1b; aNY-TCRb/a) (SEQ ID NO: 64).

FIG. 4 displays the amino acid sequence of NT2_anti-NY-ESO-1 betaCD28TmCytZCyt-2A-alphamuCD28TmCytmuZCyt (aNY-TCR28Z) (SEQ ID NO: 10).

FIG. 5 displays the amino acid sequence of anti-NY-ESO-1 betaCD28TmCytECyt-2A-alphamuCD28TmCytmuECyt (NT3; aNY-TCR28E) (SEQ ID NO: 11).

FIG. 6 displays an alignment of the nucleotide sequences hCD28TmCyt (SEQ ID NO: 17) and MuhCD28TmCy (SEQ ID NO: 18). Both DNA sequences encode an identical polypeptide sequence comprising the Tm and Cyt domains of a native human CD28 (CD28TmCyt) (SEQ ID NO: 6). The overall homology between these two sequences is 56%. Identical nucleotides are denoted with an asterisk (*).

FIG. 7 displays an alignment of the nucleotide sequences hCD3ZCyt(ZCyt) (SEQ ID NO: 19) and muhCD3ZCyt(muZCyt) (SEQ ID NO: 20). Both DNA sequences encode an identical polypeptide sequence comprising the Cyt domain of a native human CD3Zeta (SEQ ID NO: 7). The overall homology between these two sequences is 57%. Identical nucleotides are denoted with an asterisk (*).

FIG. 8 displays an alignment of the nucleotide sequences hCD28TmCytCD3ZCyt (28TmCytZCyt) (SEQ ID NO: 16) and muhCD28TmCytCD3ZCyt(mu28TmCytZCyt) (SEQ ID NO: 21). Both DNA sequences encode an identical polypeptide sequence comprising the Tm and Cyt domain of a native human CD28 and the Cyt of a native human CD3Zeta (SEQ ID NO: 15). The overall homology between these two sequences is 59%. Identical nucleotides are denoted with an asterisk (*).

FIG. 9 displays an alignment of the nucleotide sequences hCD3ECyt(ECyt) that contains a single silent nucleotide mutation (G to C) at position 119 of human CD3E (Genbank no: NM_000733.1) (SEQ ID NO: 23) to disrupt the BspE 1 site (SEQ ID NO: 23) and muhCD3ECyt(muECyt) (SEQ ID NO: 24). Both DNA sequences encode an identical polypeptide sequence comprising the Cyt domain of a native human CD3 Epsilon Chain (CD3E) (ECyt) (SEQ ID NO: 8). The overall homology between these two sequences is 57%. Identical nucleotides are denoted with an asterisk (*).

FIG. 10 shows an alignment of nucleotide sequences of Cyt of CD3Z with mutated Cyt of CD3Z. The CD3ZCyt sequence (SEQ ID NO: 19.) is from human CD3 Zeta (GenBank ID: J04132.1). The sequences of hCD3ZCyte and mu2hCD3ZCyt (SEQ ID NO: 20.) encode an identical amino acid sequence (SEQ ID NO: 7). The vertical bar “|” indicates the identical nucleotides between the two sequences. The overall homology between these two sequences is 60.42%.

FIG. 11 shows an alignment of nucleotide sequences of Tm of CD28 with mutated Tm of CD28. The CD28Tm sequence (SEQ ID NO: 44.) is from human CD28 (GenBank ID: BC093698.1). The sequences of CD28Tm and mu2CD28Tm (SEQ ID NO: 45.) encode an identical amino acid sequence (SEQ ID NO: 27.). The vertical bar “|” indicates the identical nucleotides between the two sequences. The overall homology between these two sequences is 62.96%.

FIG. 12 shows an alignment of nucleotide sequences of Cyt of CD28 with mutated Cyt of CD28. The CD28Cyt sequence (SEQ ID NO: 46.) is from human CD28 (GenBank ID: BC093698.1). The sequences of hCD28Cyt and mu2hCD28Cyt (SEQ ID NO: 47.) encode an identical amino acid sequence (SEQ ID NO: 28.). The vertical bar “|” indicates the identical nucleotides between the two sequences. The overall homology between these two sequences is 53.65%.

FIG. 13 shows an alignment of nucleotide sequences of Cyt of 4-1BB with mutated Cyt of 4-1BB. The BBCyt sequence (SEQ ID NO: 48.) is from human 4-1BB (GenBank ID: U03397.1). The sequences of hBBCyt and muhBBCyt (SEQ ID NO: 49.) encode an identical amino acid sequence (SEQ ID NO: 29.). The vertical bar “|” indicates the identical nucleotides between the two sequences. The overall homology between these two sequences is 63.49%.

FIG. 14 displays an alignment of the nucleotide sequences hCD8hinge (SEQ ID NO: 50) and MuhCD8hinge (SEQ ID NO: 51). Both DNA sequences encode an identical polypeptide sequence comprising the hinge domain of a native human CD8 (CD8hinge) (SEQ ID NO: 30) (the amino acid sequence of 135-180 of Human CD8A (GenBank ID: NM_001768.4)). The overall homology between these two sequences is 58.70%. Identical nucleotides are denoted with a vertical bar “|”.

FIG. 15 displays an alignment of the nucleotide sequences hCD8Tm (SEQ ID NO: 52) and MuhCD8Tm (SEQ ID NO: 53). Both DNA sequences encode an identical polypeptide sequence comprising the Tm domain of a native human CD8 (CD8Tm) (SEQ ID NO: 31) (the amino acid sequence of 181-206 of Human CD8A, GenBank ID: NM_001768.4). The overall homology between these two sequences is 62.82%. Identical nucleotides are denoted with a vertical bar “|”.

FIG. 16 displays the sequence of NT4_anti-NY-ESO-1 betaCD8tmBBCytZCyt-2A-alphamuCD8TmBBCytmuZCyt (aNY-TCRBBZ) (SEQ ID NO: 32).

FIG. 17 displays the sequence of NT5_anti-NY-ESO-1 betaCD28tmBBCytZCyt-2A-alphamuCD28TmBBCytmuZCyt (aNY-TCRBBZ) (SEQ ID NO: 33).

FIG. 18 displays the sequence of NT6_anti-NY-ESO-1 betaCD28TmCytZCyt-2A-alphamuCD28TmCytmuZCyt (aNY-TCR28BBZ) (SEQ ID NO: 34).

FIG. 19 displays the amino acid sequence of NT21 (SEQ ID NO: 35).

FIG. 20 displays the amino acid sequence of NT22 (SEQ ID NO: 36).

FIG. 21 displays the amino acid sequence of NT23 (SEQ ID NO: 37).

FIG. 22 displays the amino acid sequence of NT24 (SEQ ID NO: 38).

FIG. 23 displays the amino acid sequence of NT25 (SEQ ID NO: 39).

FIG. 24 displays the amino acid sequence of NT26 (SEQ ID NO: 40).

FIG. 25 displays the amino acid sequence of NT27 (SEQ ID NO: 41).

FIGS. 26A and 26B are diagrams showing surface expression of anti-NY-ESO-1 TCR-CARs on infected PG13 VPC. The PG13 were infected with anti-NY-ESO-1 TCR-CAR+ (NT22 (Vector 9; 3^{rd} gen), NT24 (Vector 11; 3^{rd} gen), NT25 (Vector 12; 2^{nd} gen)). (A) Intensity plots showing positive staining of single or double positive cells to FITC-anti-human TCRVb13.1 and APC-NYpep/A2 tetramer, individually. % of positive cells were shown in individual quadrants. (B) Histograms shows % of cells that were stained positive to NYpep/A2 tetramer. Untd: uninfected.

FIGS. 27A and 27B are diagrams showing surface expression of anti-NY-ESO-1 TCRs of native form or TCR-CARs of 2^{nd} gen on transduced activated human T cells. The ATC that were evaluated were transduced with one of the two anti-NY ESO-1 TCR (NT1, NT1b) or one of the two 2^{nd} gen TCR-based CARs (NT2 and NT3). (A) Intensity plots showing positive staining of single or double positive cells to FITC-anti-human TCRVb13.1 and APC-NYpep/A2 tetramer, individually. Percentage of positive cells were shown in individual quadrants. (B) Histograms show percentage of cells stained positive to NYpep/A2 tetramer.

FIG. 28 is a set of photographs showing specific killing of tumor cells (Saos-2) by ATC transduced with one of the two anti-NY ESO-1 TCR (NT1, NT1b) or one of the two 2^{nd} gen TCR-based CARs (NT2 and NT3).

FIGS. 29A and 29B are diagrams showing cytokine secretion of by ATC transduced with one of the two anti-NY ESO-1 TCR (NT1, NT1b) or one of the two 2^{nd} gen TCR-based CARs (NT2 and NT3). (A) IL-2 secretion and (B) INF-γ secretion, where data are presented as the mean number±S.D. from three samples in each group.

FIG. 30 is a diagram showing changes of percentages of NYpep/A2+ cells of ATC transduced with one of the two anti-NY ESO-1 TCR (NT1 and NT1b) or one of the two 2^{nd} gen TCR-based CARs (NT2 and NT3) post co-culture with tumor target cells (Saos-2)

FIGS. 31A and 31B are diagrams showing surface expression of anti-NY-ESO-1 TCRs of a native form or TCR-CARs of 2^{nd} or 3^{rd} gen on transduced activated human T cells. The ATC that were evaluated were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen), or left untransduced (Untd)). (A) Intensity plots showing positive staining of single or double positive cells to FITC-anti-human TCRVb13.1 and APC-NYpep/A2 tetramer, individually. % of positive cells were shown in individual quadrants. (B) Histograms shows % of cells that were stained positive to NYpep/A2 tetramer (the digit numbers in the parentheses indicate the geomeans).

FIG. 32 is a diagram showing killing of target cells by human ATC transduced with anti-NY ESO-1 TCR or one of the four TCR-based CARs post engagement with target cells (Saos-2). The ATC that were evaluated were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen), or left untransduced (Untd)).

FIGS. 33A and 33B are diagrams showing (A) IL-2 secretion and (B) IFN-γ of human ATC transduced with anti-NY ESO-1 TCR or one of the four TCR-based CARs post engagement with target cells (Saos-2). The ATC that were evaluated were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen)). The ATC were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen)), or left untransduced (Untd)).

FIGS. 34A and 34B are diagrams showing changes of (A) percentages and (B) expansion of NYpep/A2+ ATC transduced with anti-NY ESO-1 TCR or one of the four TCR-based CARs post engagement with target cells (Saos-2). The ATC that were evaluated were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen)), or left untransduced (Untd)).

FIGS. 35A and 35B are diagrams showing in vivo therapeutic effects of expression of 2^{nd} gen or 3^{rd} gen TCR-CARs in possible enhanced anti-NY-ESO-1/A2 TCR CAR-mediated anti-tumor activity in human T cells in Xenograft Saos-2 tumor murine models. The ATC that were evaluated in the in vivo anti-tumor therapeutic experiment were transduced with anti-NY ESO-1 TCR (NT1 (also named NT1a); Vector 1) or anti-NY-ESO-1 TCR-CAR+ (NT2 (Vector 3; 2^{nd} gen), NT4 (Vector 5; 2^{nd} gen), NT5 (Vector 6; 2^{nd} gen), NT24 (Vector 11; 3^{rd} gen)), or left untransduced (Untd)). (A) Mean number of tumor size+S.D. from six mice in each group. (B) Percentages of survival of mice/group were calculated and presented.

### DETAILED DESCRIPTION OF THE INVENTION

The present disclosure relates to agents, methods and compositions to confer and/or increase immune responses mediated by cellular immunotherapy, such as by adoptively transferring antigen-specific genetically modified subsets of lymphocytes.

Such an adoptive cell transfer or adoptive cell therapy (ACT) represents a promising therapeutic approach for the treatment of cancer patients. However, it faces various obstacles such as loss of stability for efficient CAR expression and activity. This disclosure addresses such obstacles in a number of aspects, including but not limited to, (1) novel designs of 3^{rd} gen TCR-CARs incorporating co-stimulatory signaling of, e.g., human 4-1BB, (2) novel designs involving variations in different transmembrane domains, varied copy numbers of same TCR signaling elements (such as CD3Zeta and co-stimulatory molecules of CD28 or 4-1BB). The approaches described herein enhance TCR-CAR mediated T cell signaling to improve TCR-CAR expressing T cell's anti-tumor ability via genetically expressing novel 3^{rd} gen TCR-CAR in T cells.

The disclosure provides compositions comprising genetically modified lymphocytes that express chimeric antigen receptors having the ability to modulate the immune system and the innate and adaptive immune response. The disclosed agents, methods, and compositions provide genetically engineered lymphocytes with enhanced anti-tumor functions as well as methods of developing such lymphocytes.

### Antigen Receptors

Genetically modified immune function cells, such as T cells and NK cells engineered to express foreign antigen receptors are effective immunotherapeutic for cancer and infectious diseases. Isolation of autologous antigen specific immune cells, such as T cells, for therapeutic application is a laborious task, and is not possible where such cells are absent or rare. Therefore, strategies have been developed to genetically transfer immune receptors specific to tumor or virus into patients' T cells. To this end, antigen receptors have been constructed that join antigen (Ag)-recognition domains to signaling domains of the TCR or Fc receptor. T cells expressing such antigen receptors recapitulate the immune specific responses mediated by the introduced receptor.

Chimeric antigen receptors (also known as chimeric immunoreceptors, chimeric T cell receptors or artificial T cell receptors) are receptor proteins that have been engineered to give T cells the new ability to target a specific protein. The receptors are chimeric because they combine antigen binding and T-cell activating functions into a single receptor. In addition to antigen-binding sites, a CAR can have one or more function domains.

### Domains

As described herein, an antigen receptor contains three domains: an extracellular domain, a transmembrane domain, and a cytoplasmic domain (which can contain an intracellular signaling domain). In some embodiment, it contains a fourth domain: an extracellular hinge region between the extracellular domain and the cytoplasmic domain. As such, the chimeric antigen receptor combines many facets of normal T cell activation into a single protein. They link an extracellular antigen recognition domain to an intracellular signaling domain, which activates the T cell when an antigen is bound.

An extracellular domain comprises an antigen-binding or target-binding domain. Exposed to the outside of the cell, this domain interacts with potential target molecules and is responsible for targeting the CAR-T cell to any cell expressing a matching molecule. The antigen recognition domain typically can be derived from the variable regions of a monoclonal antibody linked together as a single-chain variable fragment (scFv) or from a TCR. In addition to antibody and TCR, other approaches can also been used to direct CAR specificity, usually taking advantage of ligand/receptor pairs that normally bind to each other. For example, cytokines, innate immune receptors, TNF receptors, growth factors, and structural proteins can be used as antigen recognition domains. In preferred embodiments, the extracellular domain comprises a variable region of antibody or functional fragment thereof, an extracellular domain of a T cell receptor (e.g., Va or Vb; VaCa or VbCb) or functional fragment thereof.

A hinge region, also called a spacer, is a small structural domain that sits between the antigen recognition region and the cell's outer membrane. An ideal hinge enhances the flexibility of the receptor's target binding domain, reducing the spatial constraints between the CAR and its target antigen. This promotes antigen binding and synapse formation between the CAR-T cells and target cells. Hinge sequences can comprise the membrane-proximal region from an immune molecule, such as IgG, CD8, or CD28.

A transmembrane domain is a structural component, consisting of a hydrophobic alpha helix that spans the cell membrane. It anchors the antigen receptor to the plasma membrane, bridging the extracellular hinge and antigen recognition domains with the cytoplasmic/intracellular signaling region. This domain is important for the stability of the receptor as a whole. In this disclosure, the transmembrane domain from the most membrane-proximal component of the cytoplasmic can be used, but different transmembrane domains may result in different receptor stability. As used herein, a transmembrane domain may comprise an art-recognized, functional transmembrane domain (e.g., that of CD3ζ, CD28, CD8, CD4, or FcεRiγ, or a variant polypeptide or functional fragment thereof). The CD28 transmembrane domain is known to result in a highly expressed, stable receptor.

An intracellular T-cell signaling domain lies in the receptor's cytoplasmic domain, inside the cell. After an antigen is bound to the external antigen recognition domain, the receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. Accordingly, the CD3-zeta's cytoplasmic domain can be used. Other ITAM-containing domains can also be used. For example, the intracellular signaling domain may comprise e.g., the cytoplasmic portion of CD35 or a functional fragment thereof, FCεRIγ or a functional fragment thereof, and/or CD28 or a functional fragment thereof.

T cells also use co-stimulatory molecules in addition to CD3 signaling in order to persist after activation. The cytoplasmic domain of a CAR receptor therefore can also include one or more chimeric domains from co-stimulatory proteins (i.e., co-stimulatory domains). Signaling domains from a wide variety of co-stimulatory molecules can be used. Examples of co-stimulatory polypeptides known to stimulate or increase an immune response via their binding include CD28, OX-40, 4-1BB, CD27, and NKG2D and their corresponding ligands, including B7-1, B7-2, OX-40L, 4-1BBL, CD70, and NKG2D ligands. Such polypeptides are present in the tumor microenvironment and activate immune responses to neoplastic cells. In various embodiments, promoting, stimulating, or agonizing pro-inflammatory polypeptides and/or their ligands via a therapeutic transgene enhances the immune response of the immunoresponsive cell. For example, CD28 co-stimulation (signal 2) during T cell activation through TCR (signal 1) results in sustained proliferation, decreased activation-induced cell death (AICD) and improved long-term lymphocyte survival. Listed in the table below are examples of domain sequences can be included in a CAR.

TABLE A

| | SEQ | | |
|---|---|---|---|
| | ID | Source | Position |
| Name | NO | GenBank ID | of aa |
| cytoplasmic domains of human CD3zeta | 7 | J04132.1 | 52-163 |
| (CD3ZCyt) | | | |
| cytoplasmic domain of human CD3 | 8 | NM_000733.1 | 153-207 |
| epsilon (CD3Ecyt) | | | |
| transmembrane domain of human CD28 | 27 | BC093698.1 | 153-179 |
| cytoplasmic domain of human CD28 | 28 | BC093698.1 | 180-220 |
| cytoplasmic domain of human 4-1BB | 29 | U03397.1 | 214-255 |
| CD8hinge of human CD8A | 30 | NM_001768.4 | 135-180 |
| transmembrane domain of human CD8A | 31 | NM_001768.4 | 181-206 |

The two primary antigen receptors used to re-target T cells are transgenic T cell receptors (tTCRs) and chimeric antigen receptors (CARs). CARs can be classified into two forms, antibody-based CARs and TCR-based CARs. Antibody-based CAR therapy has demonstrated significant success in targeting B cell leukemia, and trials targeting solid tumors are underway. While antibody-based CARs have great potential as therapeutic agents in cancer immunotherapy, they are limited in their ability to recognize cell-surface molecules only. In contrast, tTCRs and TCR-based CARs have the ability to identify any processed antigen that is presented by the major histocompatibility complex (MHC), thus greatly expanding the list of possible targets. For example, in vitro studies have demonstrated that cells engineered with endogenously occurring NY-ESO-1 TCRs have activity against NY-ESO-1-expressing melanoma and non-melanoma cell lines. In a recent clinical trial employing NY-ESO-1-directed T cells, engineered cells bearing high-affinity tTCRs were delivered to patients with melanoma and synovial cell carcinoma. Nearly half of patients in this study demonstrated objective clinical responses, highlighting the potential of tTCR T cells in treating established solid tumors.

There are two ways to introduce genetically TCR-associated antigen specificity to T cells: genetically engineering T cells to express exogenous native TCRs (e.g., TCRα and TCRβ chains) and genetically engineering T cells to express exogenous TCR-based CARs. A TCR-based CAR can be created in such a way that it comprises an extracellular domain (e.g., extracellular domain of a TCR alpha chain or a TCR beta chain), a transmembrane domain and TCR signaling element (e.g., a cytoplasmic domain of CD3 zeta chain (CD3Z) or CD3 Epsilon Chain (CD3E) with or without integration of co-stimulation signaling element such as cytoplasmic domain of CD28 or CD137 (4-1BB). One advantage of TCR-based CAR over native TCR is that it can comprise TCR signaling elements such CD3Z and CDE, and be integrated with co-stimulation signaling elements such as CD28 and CD137 (4-1BB).

CD28 co-stimulation (signal 2) during T cell activation through TCR (signal 1; e.g., CD3Z- or CD3E mediated signaling) has been shown to promote sustained T cell proliferation. To combine activation and co-stimulatory functions within a single receptor, CARs can be constructed that are comprised of both CD3Zeta and CD28 sequences in the same molecule. Such IgCD28Z molecules have been demonstrated to possess superior function in T cells for cytotoxicity, proliferation, and IL2 and IFNγ production.

The extracellular portion of an antibody-based CAR (Antibody-based CAR; sFv-CAR) consists of a single chain Fv or a fragment thereof. Similar to sFv-CAR, a TCR-CAR can comprise TCR variable antigen binding fragments (Vα and Vβ, or antigen binding fragments thereof) in the context of specific MHC (HLA) molecules, linked to signaling domains of the TCR or Fc receptor. TCR-CAR can be constructed in two basic forms: as a single chain TCR-CAR (scTCR-CAR), or as a two chain TCR-CAR (tcTCR-CAR). For a scTCR-CAR, both TCR Vα and Vβ are present within the same single chain TCR protein, while in a tcTCR-CAR, the Vα and Vβ are in separate chains which form heterodimers.

It is known that TCRs have around 100- to 1,000-fold lower binding affinities for their cognate antigens (peptide/MHC complex), as compared to antibodies directed to the same cognate antigens. In addition, it has been reported that the single chain format of T cell receptors generally have greatly reduced affinities for cognate antigens, as compared to the affinities of their corresponding parental two chain forms for such antigens, with some single chain T cell receptors completely losing antigen binding affinity. Higher binding affinities of TCRs, including chimeric TCRs, have been shown to be associated with higher potencies of modified T cells expressing such receptors, with regard to the function of such cells in T cell activation, including induction of proliferation, killing of target cells and induction of cytokine secretion. Several reports have indicated that single chain formats of TCR-CARs possess binding affinities that are too low to be of therapeutic or diagnostic value. Therefore, to create functional TCR-CARs with reasonably high antigen binding affinities, the two-chain format for natural TCRs possesses significant advantages over a single-chain format.

Recombination between nucleic acids is a well-established phenomenon in molecular biology. Genetic recombination that requires strong sequence homology between participating nucleic acid sequences to occur is generally referred to as homologous recombination. While most genetic knockout strategies employ homologous recombination to achieve a targeted knockout, in certain systems the occurrence of genetic recombination can impact genetic manipulations detrimentally. In particular, homologous recombination events can adversely impact construction and production of vectors, particularly viral vectors (e.g., adenovirus, retrovirus, adeno-associated virus, herpes virus, etc.), where it is often desirable to maintain highly homologous sequences (e.g., identical polypeptide sequences) within a single, stable viral vector free of homologous recombination during, e.g., passage and/or propagation of viral vector through one or more host cells and/or organisms.

Two protein molecules comprising highly homologous polypeptide sequences that are encoded by similarly highly homologous nucleic acid sequences can be transduced to a single cell using a two-vector approach as a means of reducing possible viral recombination events. For example, TCR-CARs of two-chain format, which consist of various VaCa and VbCb, respectively, but share identical signaling element polypeptide sequences (e.g., cytoplasmic domains of CD28 and/or CD3Z.). The two different vectors, each encoding one of the two homologous proteins of interest, might be generated from separate VPCs. However, the successful transduction rate of a single vector into mammalian cells, such as activated T cells exposed to a retrovirus, is often limited.

A novel approach for overcoming such problems associated with low host cell transduction efficiencies is to enable delivery of two or more nucleic acid sequences encoding for highly homologous (e.g., identical) polypeptides on a single viral vector. Such an approach allows one to produce viral vector sequences comprising nucleic acid sequences encoding two or more highly homologous (e.g., identical) polypeptides or polypeptide domains thereof. The viral vector sequences possess reduced risk of homologous recombination between such nucleic acid sequences, even during, e.g., extended passage in host cells and/or multiple infection, chromosomal integration, and/or excision events. Such reduced rates of homologous recombination are attributable, at least in part, to exploitation of the degeneracy of the genetic code during synthesis of the viral vectors as described herein.

In addition, the hinge/spacer region consists of a CAR's non-antigen binding extracellular region. The hinge/spacer region modulates CAR function by providing flexibility, extending the length, allowing dimerization to occur, or improving stability. These properties have been suggested to influence the effector cell-target cell interactions, thereby affecting the activation signal strength. Common hinge/spacers regions have made use of immunoglobulin Fc, CD8α and CD28 spacer regions.

Listed below are examples of domain sequences can be included in a CAR disclosed herein and related nucleic acid sequences.

SEQ ID NO: 1. Alpha chain of 1G4 a95LY TCR (275 aa)
METLLGLLILWLQLQWVSSKQEVTQIPAALSVPEGENLVLNCSFTDSAIYNLQWFRQDPGKGLTSLLLIQSSQRE
QTSGRLNASLDKSSGRSTLYIAASQPGDSATYLCAVRPLYGGSYIPTFGRGTSLIVHPYIQNPDPAVYQLRDSKS
SDKSVCLFTDFDSQTNVSQSKDSDVYITDKTVLDMRSMDFKSNSAVAWSNKSDFACANAFNNSIIPEDTFFPSPE
SSCDVKLVEKSFETDTNLNFQNLSVIGFRILLLKVAGFNLLMTLRLWSSR
SEQ ID NO: 2. Beta chain of 1G4 a95LY TCR (311 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADCGFTSESYQQGVLSATILYEILLGKATLYAVLVSALVL
MAMVKRKDSRG
SEQ ID NO: 3. P2A (19 aa)
ATNFSLLKQAGDVEENPGP
SEQ ID NO: 4. Ec domain of alpha chain of 1G4 a95LY TCR (227 aa):
METLLGLLILWLQLQWVSSKQEVTQIPAALSVPEGENLVLNCSFTDSAIYNLQWFRQDPGKGLTSLLLIQSSQRE
QTSGRLNASLDKSSGRSTLYIAASQPGDSATYLCAVRPLYGGSYIPTFGRGTSLIVHPYIQNPDPAVYQLRDSKS
SDKSVCLFTDFDSQTNVSQSKDSDVYITDKTVLDMRSMDFKSNSAVAWSNKSDFACANAFNNSIIPEDTFFPSPE
SS
SEQ ID NO: 5. Ec domain of beta chain of 1G4 a95LY TCR (262 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRAD
SEQ ID NO: 6. Human CD28TmCyt (68 aa)
FWVLVVVGGVLACYSLLVTVAFIIFWVRSKRSRLLHSDYMNMTPRRPGPTRKHYQPYAPPRDFAAYRS
SEQ ID NO: 7. Human CD3ZCyt (112 aa)
RVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 8. Human CD3ECyt (55 aa)
KNRKAKAKPVTRGAGAGGRQRGQNKERPPPVPNPDYEPIRKGQRDLYSGLNQRRI
SEQ ID NO: 9. NT1_aNY-TCR (612 aa)
METLLGLLILWLQLQWVSSKQEVTQIPAALSVPEGENLVLNCSFTDSAIYNLQWFRQDPGKGLTSLLLIQSSQRE
QTSGRLNASLDKSSGRSTLYIAASQPGDSATYLCAVRPLYGGSYIPTFGRGTSLIVHPYIQNPDPAVYQLRDSKS
SDKSVCLFTDFDSQTNVSQSKDSDVYITDKTVLDMRSMDFKSNSAVAWSNKSDFACANAFNNSIIPEDTFFPSPE
SSCDVKLVEKSFETDTNLNFQNLSVIGFRILLLKVAGFNLLMTLRLWSSRAKRSGSGATNFSLLKQAGDVEENPG
PMSIGLLCCAALSLLWAGPVNAGVTQTPKFQVLKTGQSMTLQCAQDMNHEYMSWYRQDPGMGLRLIHYSVGAGIT
DQGEVPNGYNVSRSTTEDFPLRLLSAAPSQTSVYFCASSYVGNTGELFFGEGSRLTVLEDLKNVFPPEVAVFEPS
EAEISHTQKATLVCLATGFYPDHVELSWWVNGKEVHSGVSTDPQPLKEQPALNDSRYALSSRLRVSATFWQDPRN
HFRCQVQFYGLSENDEWTQDRAKPVTQIVSAEAWGRADCGFTSESYQQGVLSATILYEILLGKATLYAVLVSALV
LMAMVKRKDSRG
SEQ ID NO: 64. NT1b_Xho_NY-ESO.txt.xprt (612 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADCGFTSESYQQGVLSATILYEILLGKATLYAVLVSALVL
MAMVKRKDSRGAKRSGSGATNFSLLKQAGDVEENPGPMETLLGLLILWLQLQWVSSKQEVTQIPAALSVPEGENL
VLNCSFTDSAIYNLQWFRQDPGKGLTSLLLIQSSQREQTSGRLNASLDKSSGRSTLYIAASQPGDSATYLCAVRP
LYGGSYIPTFGRGTSLIVHPYIQNPDPAVYQLRDSKSSDKSVCLFTDFDSQTNVSQSKDSDVYITDKTVLDMRSM
DFKSNSAVAWSNKSDFACANAFNNSIIPEDTFFPSPESSCDVKLVEKSFETDTNLNFQNLSVIGFRILLLKVAGF
NLLMTLRLWSSR
SEQ ID NO: 10. NT2_aNY-TCR28Z (NYESO1-TCRba28Z_Xho-NY-ESO1
bmu2CD28tmcytCD3Zcyt-P2A-1g4TCRa95LYCD28tmcytCD3Zcyt), 883 aa
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPRAKRS
GSGATNFSLLKQAGDVEENPGPMETLLGLLILWLQLQWVSSKQEVTQIPAALSVPEGENLVLNCSFTDSAIYNLQ
WFRQDPGKGLTSLLLIQSSQREQTSGRLNASLDKSSGRSTLYIAASQPGDSATYLCAVRPLYGGSYIPTFGRGTS
LIVHPYIQNPDPAVYQLRDSKSSDKSVCLFTDFDSQTNVSQSKDSDVYITDKTVLDMRSMDFKSNSAVAWSNKSD
FACANAFNNSIIPEDTFFPSPESSGSPKFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 11. NT3_aNY-TCR28E (NYESO1-TCRba28E_NY-
ESO1bmu2CD28tmcytCD3Ecyt-P2A-1g4TCRa95LYCD28tmcytCD3Ecyt) (769 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSKNRKAKAKPVTRGAGAGGRQRGQNKERPPPVPNPDYEPIRK
GQRDLYSGLNQRRIAKRSGSGATNFSLLKQAGDVEENPGPMETLLGLLILWLQLQWVSSKQEVTQIPAALSVPEG
ENLVLNCSFTDSAIYNLQWFRQDPGKGLTSLLLIQSSQREQTSGRLNASLDKSSGRSTLYIAASQPGDSATYLCA
VRPLYGGSYIPTFGRGTSLIVHPYIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSGSPKFWVLVVVGGVLACYSLLVTVAFIIFWVRS
KRSRLLHSDYMNMTPRRPGPTRKHYQPYAPPRDFAAYRSKNRKAKAKPVTRGAGAGGRQRGQNKERPPPVPNPDY
EPIRKGQRDLYSGLNQRRI
SEQ ID NO: 12. Nucleotide sequence encoding NT1_aNY-TCR (NYESO1-TCRab_Xho-
NY-ESO11g4TCRa95LY-P2A-b-Not)
ATGGAGACCCTCTTGGGCCTGCTTATCCTTTGGCTGCAGCTGCAATGGGTGAGCAGCAAACAGGAGGTGACGCAG
ATTCCTGCAGCTCTGAGTGTCCCAGAAGGAGAAAACTTGGTTCTCAACTGCAGTTTCACTGATAGCGCTATTTAC
AACCTCCAGTGGTTTAGGCAGGACCCTGGGAAAGGTCTCACATCTCTGTTGCTTATTCAGTCAAGTCAGAGAGAG
CAAACAAGTGGAAGACTTAATGCCTCGCTGGATAAATCATCAGGACGTAGTACTTTATACATTGCAGCTTCTCAG
CCTGGTGACTCAGCCACCTACCTCTGTGCTGTGAGGCCCCTGTACGGAGGAAGCTACATACCTACATTTGGAAGA
GGAACCAGCCTTATTGTTCATCCGTATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTG
TATATCACAGACAAAACTGTGCTAGACATGAGGTCTATGGACTTCAAGAGCAACAGTGCTGTGGCCTGGAGCAAC
AAATCTGACTTTGCATGTGCAAACGCCTTCAACAACAGCATTATTCCAGAAGACACCTTCTTCCCCAGCCCAGAA
AGTTCCTGTGATGTCAAGCTGGTCGAGAAAAGCTTTGAAACAGATACGAACCTAAACTTTCAAAACCTGTCAGTG
ATTGGGTTCCGAATCCTCCTCCTGAAAGTGGCCGGGTTTAATCTGCTCATGACGCTGCGGCTGTGGTCCAGCCGG
GCCAAGCGGTCTGGGTCTGGGGCCACCAACTTCAGCCTGCTGAAGCAGGCCGGCGACGTGGAGGAGAACCCCGGC
CCCATGAGCATCGGCCTCCTGTGCTGTGCAGCCTTGTCTCTCCTGTGGGCAGGTCCAGTGAATGCTGGTGTCACT
CAGACCCCAAAATTCCAGGTCCTGAAGACAGGACAGAGCATGACACTGCAGTGTGCCCAGGATATGAACCATGAA
TACATGTCCTGGTATCGACAAGACCCAGGCATGGGGCTGAGGCTGATTCATTACTCAGTTGGTGCTGGTATCACT
GACCAAGGAGAAGTCCCCAATGGCTACAATGTCTCCAGATCAACCACAGAGGATTTCCCGCTCAGGCTGCTGTCG
GCTGCTCCCTCCCAGACATCTGTGTACTTCTGTGCCAGCAGTTACGTCGGGAACACCGGGGAGCTGTTTTTTGGA
GAAGGCTCTAGGCTGACCGTACTGGAGGACCTGAAAAACGTGTTCCCACCCGAGGTCGCTGTGTTTGAGCCATCA
GAAGCAGAGATCTCCCACACCCAAAAGGCCACACTGGTGTGCCTGGCCACAGGCTTCTACCCCGACCACGTGGAG
CTGAGCTGGTGGGTGAATGGGAAGGAGGTGCACAGTGGGGTCAGCACAGACCCGCAGCCCCTCAAGGAGCAGCCC
GCCCTCAATGACTCCAGATACGCTCTGAGCAGCCGCCTGAGGGTCTCGGCCACCTTCTGGCAGGACCCCCGCAAC
CACTTCCGCTGTCAAGTCCAGTTCTACGGGCTCTCGGAGAATGACGAGTGGACCCAGGATAGGGCCAAACCCGTC
ACCCAGATCGTCAGCGCCGAGGCCTGGGGTAGAGCAGACTGTGGCTTCACCTCCGAGTCTTACCAGCAAGGGGTC
CTGTCTGCCACCATCCTCTATGAGATCTTGCTAGGGAAGGCCACCTTGTATGCCGTGCTGGTCAGTGCCCTCGTG
CTGATGGCTATGGTCAAGAGAAAGGATTCCAGAGGCTAA
SEQ ID NO: 65. NT1b_Xho_NY-ESO1-TCRb-P2A-1g4TCRa95LY-not.txt.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACTGTGGCTTCACCTCCGAGTCTTACCAGCAAGGGGTCCTG
TCTGCCACCATCCTCTATGAGATCTTGCTAGGGAAGGCCACCTTGTATGCCGTGCTGGTCAGTGCCCTCGTGCTG
ATGGCTATGGTCAAGAGAAAGGATTCCAGAGGCGCCAAGCGGTCTGGGTCTGGGGCCACCAACTTCAGCCTGCTG
AAGCAGGCCGGCGACGTGGAGGAGAACCCCGGCCCCATGGAGACCCTCTTGGGCCTGCTTATCCTTTGGCTGCAG
CTGCAATGGGTGAGCAGCAAACAGGAGGTGACGCAGATTCCTGCAGCTCTGAGTGTCCCAGAAGGAGAAAACTTG
GTTCTCAACTGCAGTTTCACTGATAGCGCTATTTACAACCTCCAGTGGTTTAGGCAGGACCCTGGGAAAGGTCTC
ACATCTCTGTTGCTTATTCAGTCAAGTCAGAGAGAGCAAACAAGTGGAAGACTTAATGCCTCGCTGGATAAATCA
TCAGGACGTAGTACTTTATACATTGCAGCTTCTCAGCCTGGTGACTCAGCCACCTACCTCTGTGCTGTGAGGCCC
CTGTACGGAGGAAGCTACATACCTACATTTGGAAGAGGAACCAGCCTTATTGTTCATCCGTATATCCAGAACCCT
GACCCTGCCGTGTACCAGCTGAGAGACTCTAAATCCAGTGACAAGTCTGTCTGCCTATTCACCGATTTTGATTCT
CAAACAAATGTGTCACAAAGTAAGGATTCTGATGTGTATATCACAGACAAAACTGTGCTAGACATGAGGTCTATG
GACTTCAAGAGCAACAGTGCTGTGGCCTGGAGCAACAAATCTGACTTTGCATGTGCAAACGCCTTCAACAACAGC
ATTATTCCAGAAGACACCTTCTTCCCCAGCCCAGAAAGTTCCTGTGATGTCAAGCTGGTCGAGAAAAGCTTTGAA
ACAGATACGAACCTAAACTTTCAAAACCTGTCAGTGATTGGGTTCCGAATCCTCCTCCTGAAAGTGGCCGGGTTT
AATCTGCTCATGACGCTGCGGCTGTGGTCCAGCCGGTAA
SEQ ID NO: 13. Nucleotide sequence encoding NT2_aNY-TCR28Z
NYESO1-TCRba28Z_Xho-NY-ESO1 bmu2CD28tmcytCD3Zcyt-P2A-
1g4TCRa95LYCD28tmcytCD3Zcyt-Not
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTCGGGTCAAATTTTCACGCTCCGCTGATGCTCCTGCCTATCAACAAGGG
CAAAATCAATTGTACAATGAATTGAACTTGGGTAGAAGGGAAGAATATGACGTGCTCGATAAACGGAGGGGGAGA
GATCCAGAAATGGGCGGTAAACCACGGCGCAAAAATCCACAAGAGGGATTGTATAACGAGCTCCAAAAGGACAAA
ATGGCAGAAGCTTATTCAGAAATAGGAATGAAGGGGGAAAGGAGACGAGGTAAAGGTCATGACGGATTGTATCAA
GGATTGTCAACCGCTACTAAAGATACATATGATGCTTTGCATATGCAAGCTTTGCCTCCCAGAGCCAAGCGGTCT
GGGTCTGGGGCCACCAACTTCAGCCTGCTGAAGCAGGCCGGCGACGTGGAGGAGAACCCCGGCCCCATGGAGACC
CTCTTGGGCCTGCTTATCCTTTGGCTGCAGCTGCAATGGGTGAGCAGCAAACAGGAGGTGACGCAGATTCCTGCA
GCTCTGAGTGTCCCAGAAGGAGAAAACTTGGTTCTCAACTGCAGTTTCACTGATAGCGCTATTTACAACCTCCAG
TGGTTTAGGCAGGACCCTGGGAAAGGTCTCACATCTCTGTTGCTTATTCAGTCAAGTCAGAGAGAGCAAACAAGT
GGAAGACTTAATGCCTCGCTGGATAAATCATCAGGACGTAGTACTTTATACATTGCAGCTTCTCAGCCTGGTGAC
TCAGCCACCTACCTCTGTGCTGTGAGGCCCCTGTACGGAGGAAGCTACATACCTACATTTGGAAGAGGAACCAGC
CTTATTGTTCATCCGTATATCCAGAACCCTGACCCTGCCGTGTACCAGCTGAGAGACTCTAAATCCAGTGACAAG
TCTGTCTGCCTATTCACCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGAAAGTTCCGGC
TCCCCAAAATTTTGGGTGCTGGTGGTGGTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCTATCGCTCCAGAGTGAAGTTC
AGCAGGAGCGCAGACGCCCCCGCGTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAGGACACCTACGACGCCCTT
CACATGCAGGCCCTGCCCCCTCGCTAA
SEQ ID NO: 14. Nucleotide sequence encoding NT3_aNY-TCR28E
NYESO1-TCRba28E_Xho-NY-ESO1 bmu2CD28tmcytCD3Ecyt-P2A-
1g4TCRa95LYCD28tmcytCD3Ecyt-Not
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTAAAAACCGCAAAGCTAAAGCTAAACCCGTCACTAGGGGGGCCGGAGCA
GGAGGGCGCCAGCGCGGTCAGAATAAAGAACGCCCTCCTCCCGTCCCTAATCCTGATTACGAACCGATTAGAAAG
GGGCAAAGAGATCTCTACAGCGGACTCAACCAACGGAGAATTGCCAAGCGGTCTGGGTCTGGGGCCACCAACTTC
AGCCTGCTGAAGCAGGCCGGCGACGTGGAGGAGAACCCCGGCCCCATGGAGACCCTCTTGGGCCTGCTTATCCTT
TGGCTGCAGCTGCAATGGGTGAGCAGCAAACAGGAGGTGACGCAGATTCCTGCAGCTCTGAGTGTCCCAGAAGGA
GAAAACTTGGTTCTCAACTGCAGTTTCACTGATAGCGCTATTTACAACCTCCAGTGGTTTAGGCAGGACCCTGGG
AAAGGTCTCACATCTCTGTTGCTTATTCAGTCAAGTCAGAGAGAGCAAACAAGTGGAAGACTTAATGCCTCGCTG
GATAAATCATCAGGACGTAGTACTTTATACATTGCAGCTTCTCAGCCTGGTGACTCAGCCACCTACCTCTGTGCT
GTGAGGCCCCTGTACGGAGGAAGCTACATACCTACATTTGGAAGAGGAACCAGCCTTATTGTTCATCCGTATATC
CAGAACCCTGACCCTGCCGTGTACCAGCTGAGAGACTCTAAATCCAGTGACAAGTCTGTCTGCCTATTCACCGAT
TTTGATTCTCAAACAAATGTGTCACAAAGTAAGGATTCTGATGTGTATATCACAGACAAAACTGTGCTAGACATG
AGGTCTATGGACTTCAAGAGCAACAGTGCTGTGGCCTGGAGCAACAAATCTGACTTTGCATGTGCAAACGCCTTC
AACAACAGCATTATTCCAGAAGACACCTTCTTCCCCAGCCCAGAAAGTTCCGGCTCCCCAAAATTTTGGGTGCTG
GTGGTGGTTGGTGGAGTCCTGGCTTGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGCCCCGGGCCCACCCGCAAGCATTAC
CAGCCCTATGCCCCACCACGCGACTTCGCAGCCTATCGCTCCAAGAATAGAAAGGCCAAGGCCAAGCCTGTGACA
CGAGGAGCGGGTGCTGGCGGCAGGCAAAGGGGACAAAACAAGGAGAGGCCACCACCTGTTCCCAACCCAGACTAT
GAGCCCATCCGCAAAGGCCAGCGGGACCTGTATTCTGGCCTGAATCAGAGACGCATCTAA
SEQ ID NO: 15. hCD28TmCytCD3ZCyt (141 aa)
MNMTPRRPGPTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPP
R
SEQ ID NO: 16. Nucleotide sequence encoding hCD28TmCytCD3ZCyt (540 bp)
TTTTGGGTGCTGGTGGTGGTTGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGACTCCCCGCCGCCC
CGGGCCCACCCGCAAGCATTACCAGCCCTATGCCCCACCACGCGACTTCGCAGCCTATCGCTCCAGAGTG
AAGTTCAGCAGGAGCGCAGACGCCCCCGCGTACCAGCAGGGCCAGAACCAGCTCTATAACGAGCTCAATC
TAGGACGAAGAGAGGAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCTCAGTACAG
CCACCAAGGACACCTACGACGCCCTTCACATGCAGGCCCTGCCCCCTCGC
SEQ ID NO: 17. Nucleotide sequence encoding hCD28TmCyt (204 bp)
TTTTGGGTGCTGGTGGTGGTTGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGACTCCCCGCCGCCC
CGGGCCCACCCGCAAGCATTACCAGCCCTATGCCCCACCACGCGACTTCGCAGCCTATCGCTCC
SEQ ID NO: 18. Nutated nucleotide sequence encoding hCD28TmCytCD3ZCyt
(mu28TmCyt) (204 bp)
TTCTGGGTTCTCGTCGTCGTGGGAGGTGTGTTAGCATGTTACTCTCTCTTGGTTACTGTCGCTTTCATAATCTTT
TGGGTCCGCTCAAAACGCTCTCGCTTGTTACATTCCGATTATATGAATATGACACCTAGGAGACCTGGCCCGACT
AGGAAACACTATCAACCTTACGCACCTCCCAGAGATTTTGCTGCTTACAGGAGT
SEQ ID NO: 19. Nucleotide sequence encoding human CD3ZCyt (ZCyt), 336 bp
AGAGTGAAGTTCAGCAGGAGCGCAGACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCAGTACAGCCACCAAGGACACC
TACGACGCCCTTCACATGCAGGCCCTGCCCCCTCGC
SEQ ID NO: 20. Mutated nucleotide sequence encoding human CD3ZCyt (muZCyt),
muhCD3ZCyt, 336 bp
CGGGTCAAATTTTCACGCTCCGCTGATGCTCCTGCCTATCAACAAGGGCAAAATCAATTGTACAATGAATTGAAC
TTGGGTAGAAGGGAAGAATATGACGTGCTCGATAAACGGAGGGGGAGAGATCCAGAAATGGGCGGTAAACCACGG
CGCAAAAATCCACAAGAGGGATTGTATAACGAGCTCCAAAAGGACAAAATGGCAGAAGCTTATTCAGAAATAGGA
ATGAAGGGGGAAAGGAGACGAGGTAAAGGTCATGACGGATTGTATCAAGGATTGTCAACCGCTACTAAAGATACA
TATGATGCTTTGCATATGCAAGCTTTGCCTCCCAGA
SEQ ID NO: 21. Mutated nucleotide sequence encoding hCD28TmCytCD3ZCyt
(mu28TmCytmuCyt)muhCD28TmCytmuCD3ZCyt, 540 bp
TTCTGGGTTCTCGTCGTCGTGGGAGGTGTGTTAGCATGTTACTCTCTCTTGGTTACTGTCGCTTTCATAATCTTT
TGGGTCCGCTCAAAACGCTCTCGCTTGTTACATTCCGATTATATGAATATGACACCTAGGAGACCTGGCCCGACT
AGGAAACACTATCAACCTTACGCACCTCCCAGAGATTTTGCTGCTTACAGGAGTCGGGTCAAATTTTCACGCTCC
GCTGATGCTCCTGCCTATCAACAAGGGCAAAATCAATTGTACAATGAATTGAACTTGGGTAGAAGGGAAGAATAT
GACGTGCTCGATAAACGGAGGGGGAGAGATCCAGAAATGGGCGGTAAACCACGGCGCAAAAATCCACAAGAGGGA
TTGTATAACGAGCTCCAAAAGGACAAAATGGCAGAAGCTTATTCAGAAATAGGAATGAAGGGGGAAAGGAGACGA
GGTAAAGGTCATGACGGATTGTATCAAGGATTGTCAACCGCTACTAAAGATACATATGATGCTTTGCATATGCAA
GCTTTGCCTCCCAGA
SEQ ID NO: 22. Nucleotide sequence of human CD3ECyt_NM_000733.1, (165 bp)
AAGAATAGAAAGGCCAAGGCCAAGCCTGTGACACGAGGAGCGGGTGCTGGCGGCAGGCAAAGGGGACAAAACAAG
GAGAGGCCACCACCTGTTCCCAACCCAGACTATGAGCCCATCCGGAAAGGCCAGCGGGACCTGTATTCTGGCCTG
AATCAGAGACGCATC
SEQ ID NO: 23. Nucleotide sequence of human CD3ECyt_NM_000733.1 with a single
silent nucleotide mutation (G to C) at position 119 to disrupt the BspE 1 site,
(165 bp)
AAGAATAGAAAGGCCAAGGCCAAGCCTGTGACACGAGGAGCGGGTGCTGGCGGCAGGCAAAGGGGACAAAACAAG
GAGAGGCCACCACCTGTTCCCAACCCAGACTATGAGCCCATCCGCAAAGGCCAGCGGGACCTGTATTCTGGCCTG
AATCAGAGACGCATC
SEQ ID NO: 24. Nucleotide sequence of silently mutated human
CD3ECyt_NM_000733.1, edited, 165 bp
AAAAACCGCAAAGCTAAAGCTAAACCCGTCACTAGGGGGGCCGGAGCAGGAGGGCGCCAGCGCGGTCAGAATAAA
GAACGCCCTCCTCCCGTCCCTAATCCTGATTACGAACCGATTAGAAAGGGGCAAAGAGATCTCTACAGCGGACTC
AACCAACGGAGAATT
SEQ ID NO: 25. Human NY-ESO-1 peptide corresponding to residues 157 to 165 of
human NY-ESO-1 (NY-ESO-1: 157-165) (9 aa)
SLLMWITQC
SEQ ID NO: 26. CD28pec (40 aa)
KIEVMYPPPYLDNEKSNGTIIHVKGKHLCPSPLFPGPSKP
SEQ ID NO: 27. Human CD28Tm
FWVLVVVGGVLACYSLLVTVAFIIFWV
SEQ ID NO: 28. Human CD28Cyt
RSKRSRLLHSDYMNMTPRRPGPTRKHYQPYAPPRDFAAYRS
SEQ ID NO: 29. Human 4-1BBCyt
KRGRKKLLYIFKQPFMRPVQTTQEEDGCSCRFPEEEEGGCEL
SEQ ID NO: 30. CD8hinge.txt.xprt (46 aa)
AKPTTTPAPRPPTPAPTIASQPLSLRPEACRPAAGGAVHTRGLDFA
SEQ ID NO: 31. CD8Tm_aa 181-20.txt.xprt (26 aa)
CDIYIWAPLAGTCGVLLLSLVITLYC
SEQ ID NO: 32. NT4_NYESO1-TCRbaBBZ_Xho-NY-ESO1 bmuCD8tmBBcyt
CD3Zcyt-P2A-1g4TCRa95LYCCD8tmBBcytZcyt-Not.txt.xprt, (883 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKCDIYIWAPLAGTCGVLLLSLVITLYCKRGRKKLL
YIFKQPFMRPVQTTQEEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPRAKRS
GSGATNFSLLKQAGDVEENPGPMETLLGLLILWLQLQWVSSKQEVTQIPAALSVPEGENLVLNCSFTDSAIYNLQ
WFRQDPGKGLTSLLLIQSSQREQTSGRLNASLDKSSGRSTLYIAASQPGDSATYLCAVRPLYGGSYIPTFGRGTS
LIVHPYIQNPDPAVYQLRDSKSSDKSVCLFTDFDSQTNVSQSKDSDVYITDKTVLDMRSMDFKSNSAVAWSNKSD
FACANAFNNSIIPEDTFFPSPESSGSPKCDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 33. NT5_Design_NYE.txt.xprt (885 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVKRGRKKL
LYIFKQPFMRPVQTTQEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPRAKR
SGSGATNFSLLKQAGDVEENPGPMETLLGLLILWLQLQWVSSKQEVTQIPAALSVPEGENLVLNCSFTDSAIYNL
QWFRQDPGKGLTSLLLIQSSQREQTSGRLNASLDKSSGRSTLYIAASQPGDSATYLCAVRPLYGGSYIPTFGRGT
SLIVHPYIQNPDPAVYQLRDSKSSDKSVCLFTDFDSQTNVSQSKDSDVYITDKTVLDMRSMDFKSNSAVAWSNKS
DFACANAFNNSIIPEDTFFPSPESSGSPKFWVLVVVGGVLACYSLLVTVAFIIFWVKRGRKKLLYIFKQPFMRPV
QTTQEEDGCSCRFPEEEEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR
SEQ ID NO: 34. NT6_NYESO1-TCRb.txt.xprt (967 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSKRGRKKLLYIFKQPFMRPVQTTQEEDGCSCRFPEEEEGGCE
LRVKFSRSADAPAYQQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPRAKRSGSGATNFSLLKQAGDVEENPGPMETLLGLLILW
LQLQWVSSKQEVTQIPAALSVPEGENLVLNCSFTDSAIYNLQWFRQDPGKGLTSLLLIQSSQREQTSGRLNASLD
KSSGRSTLYIAASQPGDSATYLCAVRPLYGGSYIPTFGRGTSLIVHPYIQNPDPAVYQLRDSKSSDKSVCLFTDF
DSQTNVSQSKDSDVYITDKTVLDMRSMDFKSNSAVAWSNKSDFACANAFNNSIIPEDTFFPSPESSGSPKFWVLV
VVGGVLACYSLLVTVAFIIFWVRSKRSRLLHSDYMNMTPRRPGPTRKHYQPYAPPRDFAAYRSKRGRKKLLYIFK
QPFMRPVQTTQEEDGCSCRFPEEEEGGCELRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYDALHMQALPPR
SEQ ID NO: 35. NT21_.txt.xprt (883 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPRAKRS
GSGATNFSLLKQAGDVEENPGPMETLLGLLILWLQLQWVSSKQEVTQIPAALSVPEGENLVLNCSFTDSAIYNLQ
WFRQDPGKGLTSLLLIQSSQREQTSGRLNASLDKSSGRSTLYIAASQPGDSATYLCAVRPLYGGSYIPTFGRGTS
LIVHPYIQNPDPAVYQLRDSKSSDKSVCLFTDFDSQTNVSQSKDSDVYITDKTVLDMRSMDFKSNSAVAWSNKSD
FACANAFNNSIIPEDTFFPSPESSGSPKCDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 36. NT22_.txt.xprt (771 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPRAKRS
GSGATNFSLLKQAGDVEENPGPMETLLGLLILWLQLQWVSSKQEVTQIPAALSVPEGENLVLNCSFTDSAIYNLQ
WFRQDPGKGLTSLLLIQSSQREQTSGRLNASLDKSSGRSTLYIAASQPGDSATYLCAVRPLYGGSYIPTFGRGTS
LIVHPYIQNPDPAVYQLRDSKSSDKSVCLFTDFDSQTNVSQSKDSDVYITDKTVLDMRSMDFKSNSAVAWSNKSD
FACANAFNNSIIPEDTFFPSPESSGSPKCDIYIWAPLAGTCGVLLLSLVITLYCKRGRKKLLYIFKQPFMRPVQT
TQEEDGCSCRFPEEEEGGCEL
SEQ ID NO: 37. NT23_.txt.xprt (884 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKFWVLVVVGGVLACYSLLVTVAFIIFWVRSKRSRL
LHSDYMNMTPRRPGPTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPRAKRS
GSGATNFSLLKQAGDVEENPGPMETLLGLLILWLQLQWVSSKQEVTQIPAALSVPEGENLVLNCSFTDSAIYNLQ
WFRQDPGKGLTSLLLIQSSQREQTSGRLNASLDKSSGRSTLYIAASQPGDSATYLCAVRPLYGGSYIPTFGRGTS
LIVHPYIQNPDPAVYQLRDSKSSDKSVCLFTDFDSQTNVSQSKDSDVYITDKTVLDMRSMDFKSNSAVAWSNKSD
FACANAFNNSIIPEDTFFPSPESSGSPKFWVLVVVGGVLACYSLLVTVAFIIFWVKRGRKKLLYIFKQPFMRPVQ
TTQEEDGCSCRFPEEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 38. NT24_.txt.xprt (882 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKCDIYIWAPLAGTCGVLLLSLVITLYCRSKRSRLL
HSDYMNMTPRRPGPTRKHYQPYAPPRDFAAYRSRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTYDALHMQALPPRAKRSG
SGATNFSLLKQAGDVEENPGPMETLLGLLILWLQLQWVSSKQEVTQIPAALSVPEGENLVLNCSFTDSAIYNLQW
FRQDPGKGLTSLLLIQSSQREQTSGRLNASLDKSSGRSTLYIAASQPGDSATYLCAVRPLYGGSYIPTFGRGTSL
IVHPYIQNPDPAVYQLRDSKSSDKSVCLFTDFDSQTNVSQSKDSDVYITDKTVLDMRSMDFKSNSAVAWSNKSDF
ACANAFNNSIIPEDTFFPSPESSGSPKCDIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 39. NT25_.txt.xprt (965 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKCDIYIWAPLAGTCGVLLLSLVITLYCRSKRSRLL
HSDYMNMTPRRPGPTRKHYQPYAPPRDFAAYRSRSKRSRLLHSDYMNMTPRRPGPTRKHYQPYAPPRDFAAYRSR
VKFSRSADAPAYQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPRAKRSGSGATNFSLLKQAGDVEENPGPMETLLGLLILWLQ
LQWVSSKQEVTQIPAALSVPEGENLVLNCSFTDSAIYNLQWFRQDPGKGLTSLLLIQSSQREQTSGRLNASLDKS
SGRSTLYIAASQPGDSATYLCAVRPLYGGSYIPTFGRGTSLIVHPYIQNPDPAVYQLRDSKSSDKSVCLFTDFDS
QTNVSQSKDSDVYITDKTVLDMRSMDFKSNSAVAWSNKSDFACANAFNNSIIPEDTFFPSPESSGSPKCDIYIWA
PLAGTCGVLLLSLVITLYCKRGRKKLLYIFKQPFMRPVQTTQEEDGCSCRFPEEEEGGCELKRGRKKLLYIFKQP
FMRPVQTTQEEDGCSCRFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALPPR
SEQ ID NO: 40. NT26_.txt.xprt (1059 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKAKPTTTPAPRPPTPAPTIASQPLSLRPEACRPAA
GGAVHTRGLDFAPRKIEVMYPPPYLDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRRGKGHDGLYQGLSTATKDTYDA
LHMQALPPRAKRSGSGATNFSLLKQAGDVEENPGPMETLLGLLILWLQLQWVSSKQEVTQIPAALSVPEGENLVL
NCSFTDSAIYNLQWFRQDPGKGLTSLLLIQSSQREQTSGRLNASLDKSSGRSTLYIAASQPGDSATYLCAVRPLY
GGSYIPTFGRGTSLIVHPYIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSGSPKAKPTTTPAPRPPTPAPTIASQPLSLRPEACRPAA
GGAVHTRGLDFAPRKIEVMYPPPYLDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRRGKGHDGLYQGLSTATKDTYDA
LHMQALPPR
SEQ ID NO: 41. NT27_.txt.xprt (859 aa)
MSIGLLCCAALSLLWAGPVNAGVTQTPKFQVLKTGQSMTLQCAQDMNHEYMSWYRQDPGMGLRLIHYSVGAGITD
QGEVPNGYNVSRSTTEDFPLRLLSAAPSQTSVYFCASSYVGNTGELFFGEGSRLTVLEDLKNVFPPEVAVFEPSE
AEISHTQKATLVCLATGFYPDHVELSWWVNGKEVHSGVSTDPQPLKEQPALNDSRYALSSRLRVSATFWQDPRNH
FRCQVQFYGLSENDEWTQDRAKPVTQIVSAEAWGRADGSPKAKPTTTPAPRPPTPAPTIASQPLSLRPEACRPAA
GGAVHTRGLDFAPRKIEVMYPPPYLDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRRGKGHDGLYQGLSTATKDTYDA
LHMQALPPRAKRSGSGATNFSLLKQAGDVEENPGPMETLLGLLILWLQLQWVSSKQEVTQIPAALSVPEGENLVL
NCSFTDSAIYNLQWFRQDPGKGLTSLLLIQSSQREQTSGRLNASLDKSSGRSTLYIAASQPGDSATYLCAVRPLY
GGSYIPTFGRGTSLIVHPYIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSGSPKCDIYIWAPLAGTCGVLLLSLVITLYCKRGRKKLL
YIFKQPFMRPVQTTQEEDGCSCRFPEEEEGGCEL
SEQ ID NO: 42. Nucleotide sequence of pEc of hCD28_Human CD28_complete
cds_mRNA_BC093698.1.txt.xdna
(120 bp)
AAAATTGAAGTTATGTATCCTCCTCCTTACCTAGACAATGAGAAGAGCAATGGAACCATTATCCATGTGAAAGGG
AAACACCTTTGTCCAAGTCCCCTATTTCCCGGACCTTCTAAGCCC
SEQ ID NO: 43. nt of mu2 hCD28pEc_Human CD28_complete
cds_mRNA_BC093698.1.txt.xdna (120 bp)
AAGATCGAGGTAATGTACCCACCGCCCTATCTTGATAACGAAAAATCTAACGGTACAATAATTCACGTCAAGGGC
AAGCATTTGTGCCCTTCCCCGTTGTTCCCGGGCCCAAGCAAACCG
SEQ ID NO: 44. Nucleotide sequence of CD28Tm_human CD28_BC093698.1.txt.xdna
(81 bp)
TTTTGGGTGCTGGTGGTGGTTGGTGGAGTCCTGGCTTGCTATAGCTTGCTAGTAACAGTGGCCTTTATTATTTTC
TGGGTG
SEQ ID NO: 45. Nucleotide sequence of mu2CD28Tm_human
CD28_BC093698.1.txt.xdna (81 bp)
TTCTGGGTTCTCGTCGTCGTGGGAGGTGTGTTAGCATGTTACTCTCTCTTGGTTACTGTCGCTTTCATAATCTTT
TGGGTC
SEQ ID NO: 46. Nucleotide sequence of hCD28Cyt.txt.xdna (123 bp)
AGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCC
SEQ ID NO: 47. Nucleotide sequence of mu2hCD28Cyt.txt.xdna (123 bp)
CGCTCAAAACGCTCTCGCTTGTTACATTCCGATTATATGAATATGACACCTAGGAGACCTGGCCCGACTAGGAAA
CACTATCAACCTTACGCACCTCCCAGAGATTTTGCTGCTTACAGGAGT
SEQ ID NO: 48. Nucleotide sequence of design_h4-1BBCyt.xdna (126 bp)
AAACGGGGCAGAAAGAAACTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTG
SEQ ID NO: 49. Nucleotide sequence of design_muh41BBCyt.xdna (126 bp)
AAGAGAGGGCGTAAAAAGCTGCTCTACATCTTTAAGCAGCCTTTCATGCGTCCTGTTCAGACAACACAGGAAGAG
GACGGATGCTCTTGCAGGTTCCCTGAGGAGGAGGAGGGTGGTTGCGAGCTC
SEQ ID NO: 50. Nucleotide sequence of CD8hinge (138 bp)
GCTAAGCCCACCACGACGCCAGCGCCGCGACCACCAACACCGGCGCCCACCATCGCGTCGCAGCCCCTGTCCCTG
CGCCCAGAGGCGTGCCGGCCAGCGGCGGGGGGCGCAGTGCACACGAGGGGGCTGGACTTCGCC
SEQ ID NO: 51. Nucleotide sequence of muCD8hinge (138 bp)
GCTAAGCCCACTACTACCCCAGCTCCCAGGCCTCCCACACCTGCCCCAACAATCGCCAGCCAGCCACTGTCCCTT
AGGCCCGAGGCCTGTAGGCCCGCCGCCGGAGGAGCCGTGCACACCCGCGGACTGGATTTTGCT
SEQ ID NO: 52. Nucleotide sequence of CD8Tm_aa 181-206 of
H_CD8A_NM_001768.4.xdna (78 bp)
TGTGATATCTACATCTGGGCGCCCTTGGCCGGGACTTGTGGGGTCCTTCTCCTGTCACTGGTTATCACCCTTTAC
TGC
SEQ ID NO: 53. Nucleotide sequence of MuCD8Tm, 78 bp
MuCD8Tm_aa 181-206 of H_CD8A_NM_001768.4
TGCGACATTTATATTTGGGCCCCTCTCGCTGGCACATGCGGCGTGTTGTTGCTCAGCCTCGTGATTACAC
TTTATTGT
SEQ ID NO: 54. Nucleotide sequence of NT4_Design_NYESO1-TCRbaBBZ_Xho-NY-
ESO1 bmuCD8tmBBcytCD3Zcyt-P2A-1g4TCRa95LYCCD8tmBBcytZcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTGCGACATTTATATTTGGGCCCCTCTC
GCTGGCACATGCGGCGTGTTGTTGCTCAGCCTCGTGATTACACTTTATTGTAAGAGAGGGCGTAAAAAGCTGCTC
TACATCTTTAAGCAGCCTTTCATGCGTCCTGTTCAGACAACACAGGAAGAGGACGGATGCTCTTGCAGGTTCCCT
GAGGAGGAGGAGGGTGGTTGCGAGCTCCGGGTCAAATTTTCACGCTCCGCTGATGCTCCTGCCTATCAACAAGGG
CAAAATCAATTGTACAATGAATTGAACTTGGGTAGAAGGGAAGAATATGACGTGCTCGATAAACGGAGGGGGAGA
GATCCAGAAATGGGCGGTAAACCACGGCGCAAAAATCCACAAGAGGGATTGTATAACGAGCTCCAAAAGGACAAA
ATGGCAGAAGCTTATTCAGAAATAGGAATGAAGGGGGAAAGGAGACGAGGTAAAGGTCATGACGGATTGTATCAA
GGATTGTCAACCGCTACTAAAGATACATATGATGCTTTGCATATGCAAGCTTTGCCTCCCAGAGCCAAGCGGTCT
GGGTCTGGGGCCACCAACTTCAGCCTGCTGAAGCAGGCCGGCGACGTGGAGGAGAACCCCGGCCCCATGGAGACC
CTCTTGGGCCTGCTTATCCTTTGGCTGCAGCTGCAATGGGTGAGCAGCAAACAGGAGGTGACGCAGATTCCTGCA
GCTCTGAGTGTCCCAGAAGGAGAAAACTTGGTTCTCAACTGCAGTTTCACTGATAGCGCTATTTACAACCTCCAG
TGGTTTAGGCAGGACCCTGGGAAAGGTCTCACATCTCTGTTGCTTATTCAGTCAAGTCAGAGAGAGCAAACAAGT
GGAAGACTTAATGCCTCGCTGGATAAATCATCAGGACGTAGTACTTTATACATTGCAGCTTCTCAGCCTGGTGAC
TCAGCCACCTACCTCTGTGCTGTGAGGCCCCTGTACGGAGGAAGCTACATACCTACATTTGGAAGAGGAACCAGC
CTTATTGTTCATCCGTATATCCAGAACCCTGACCCTGCCGTGTACCAGCTGAGAGACTCTAAATCCAGTGACAAG
TCTGTCTGCCTATTCACCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGAAAGTTCCGGC
TCCCCAAAATGTGATATCTACATCTGGGCGCCCTTGGCCGGGACTTGTGGGGTCCTTCTCCTGTCACTGGTTATC
ACCCTTTACTGCAAACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAGGACACCTACGACGCCCTT
CACATGCAGGCCCTGCCCCCTCGCTAA
SEQ ID NO: 55. Nucleotide sequence of NT5_Design_NYESO1-
TCRbaCD28tmBBZ_Xho-NY-ESO1 bmu2CD28tmBBcytCD3Zcyt-P2A-
1g4TCRa95LYCD28tmBBcytCD3Zcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCAAGAGAGGGCGTAAAAAGCTG
CTCTACATCTTTAAGCAGCCTTTCATGCGTCCTGTTCAGACAACACAGGAAGAGGACGGATGCTCTTGCAGGTTC
CCTGAGGAGGAGGAGGGTGGTTGCGAGCTCCGGGTCAAATTTTCACGCTCCGCTGATGCTCCTGCCTATCAACAA
GGGCAAAATCAATTGTACAATGAATTGAACTTGGGTAGAAGGGAAGAATATGACGTGCTCGATAAACGGAGGGGG
AGAGATCCAGAAATGGGCGGTAAACCACGGCGCAAAAATCCACAAGAGGGATTGTATAACGAGCTCCAAAAGGAC
AAAATGGCAGAAGCTTATTCAGAAATAGGAATGAAGGGGGAAAGGAGACGAGGTAAAGGTCATGACGGATTGTAT
CAAGGATTGTCAACCGCTACTAAAGATACATATGATGCTTTGCATATGCAAGCTTTGCCTCCCAGAGCCAAGCGG
TCTGGGTCTGGGGCCACCAACTTCAGCCTGCTGAAGCAGGCCGGCGACGTGGAGGAGAACCCCGGCCCCATGGAG
ACCCTCTTGGGCCTGCTTATCCTTTGGCTGCAGCTGCAATGGGTGAGCAGCAAACAGGAGGTGACGCAGATTCCT
GCAGCTCTGAGTGTCCCAGAAGGAGAAAACTTGGTTCTCAACTGCAGTTTCACTGATAGCGCTATTTACAACCTC
CAGTGGTTTAGGCAGGACCCTGGGAAAGGTCTCACATCTCTGTTGCTTATTCAGTCAAGTCAGAGAGAGCAAACA
AGTGGAAGACTTAATGCCTCGCTGGATAAATCATCAGGACGTAGTACTTTATACATTGCAGCTTCTCAGCCTGGT
GACTCAGCCACCTACCTCTGTGCTGTGAGGCCCCTGTACGGAGGAAGCTACATACCTACATTTGGAAGAGGAACC
AGCCTTATTGTTCATCCGTATATCCAGAACCCTGACCCTGCCGTGTACCAGCTGAGAGACTCTAAATCCAGTGAC
AAGTCTGTCTGCCTATTCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGCCTGGAGCAACAAATCT
GACTTTGCATGTGCAAACGCCTTCAACAACAGCATTATTCCAGAAGACACCTTCTTCCCCAGCCCAGAAAGTTCC
GGCTCCCCAAAATTTTGGGTGCTGGTGGTGGTTGGTGGAGTCCTGGCTTGCTATAGCTTGCTAGTAACAGTGGCC
TTTATTATTTTCTGGGTGAAACGGGGCAGAAAGAAACTCCTGTATATATTCAAACAACCATTTATGAGACCAGTA
CAAACTACTCAAGAGGAAGATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTATAACGAGCTCAATCTAGGA
CGAAGAGAGGAGTACGATGTTTTGGACAAGAGACGTGGCCGGGACCCTGAGATGGGGGGAAAGCCGAGAAGGAAG
AACCCTCAGGAAGGCCTGTACAATGAACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAGTACAGCCACCAAGGACACCTACGAC
GCCCTTCACATGCAGGCCCTGCCCCCTCGCTAA
SEQ ID NO: 56. Nucleotide sequence of NT6_NYESO1-TCRba28BBZ_Xho-NY-ESO1
bmu2CD28tmcytBBcytCD3Zcyt-P2A-1g4TCRa95LYCD28tmcytBBcytCD3Zcyt-
Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTAAGAGAGGGCGTAAAAAGCTGCTCTACATCTTTAAGCAGCCTTTCATG
CGTCCTGTTCAGACAACACAGGAAGAGGACGGATGCTCTTGCAGGTTCCCTGAGGAGGAGGAGGGTGGTTGCGAG
CTCCGGGTCAAATTTTCACGCTCCGCTGATGCTCCTGCCTATCAACAAGGGCAAAATCAATTGTACAATGAATTG
AACTTGGGTAGAAGGGAAGAATATGACGTGCTCGATAAACGGAGGGGGAGAGATCCAGAAATGGGCGGTAAACCA
CGGCGCAAAAATCCACAAGAGGGATTGTATAACGAGCTCCAAAAGGACAAAATGGCAGAAGCTTATTCAGAAATA
GGAATGAAGGGGGAAAGGAGACGAGGTAAAGGTCATGACGGATTGTATCAAGGATTGTCAACCGCTACTAAAGAT
ACATATGATGCTTTGCATATGCAAGCTTTGCCTCCCAGAGCCAAGCGGTCTGGGTCTGGGGCCACCAACTTCAGC
CTGCTGAAGCAGGCCGGCGACGTGGAGGAGAACCCCGGCCCCATGGAGACCCTCTTGGGCCTGCTTATCCTTTGG
CTGCAGCTGCAATGGGTGAGCAGCAAACAGGAGGTGACGCAGATTCCTGCAGCTCTGAGTGTCCCAGAAGGAGAA
AACTTGGTTCTCAACTGCAGTTTCACTGATAGCGCTATTTACAACCTCCAGTGGTTTAGGCAGGACCCTGGGAAA
GGTCTCACATCTCTGTTGCTTATTCAGTCAAGTCAGAGAGAGCAAACAAGTGGAAGACTTAATGCCTCGCTGGAT
AAATCATCAGGACGTAGTACTTTATACATTGCAGCTTCTCAGCCTGGTGACTCAGCCACCTACCTCTGTGCTGTG
AGGCCCCTGTACGGAGGAAGCTACATACCTACATTTGGAAGAGGAACCAGCCTTATTGTTCATCCGTATATCCAG
AACCCTGACCCTGCCGTGTACCAGCTGAGAGACTCTAAATCCAGTGACAAGTCTGTCTGCCTATTCACCGATTTT
GATTCTCAAACAAATGTGTCACAAAGTAAGGATTCTGATGTGTATATCACAGACAAAACTGTGCTAGACATGAGG
TCTATGGACTTCAAGAGCAACAGTGCTGTGGCCTGGAGCAACAAATCTGACTTTGCATGTGCAAACGCCTTCAAC
AACAGCATTATTCCAGAAGACACCTTCTTCCCCAGCCCAGAAAGTTCCGGCTCCCCAAAATTTTGGGTGCTGGTG
GTGGTTGGTGGAGTCCTGGCTTGCTATAGCTTGCTAGTAACAGTGGCCTTTATTATTTTCTGGGTGAGGAGTAAG
AGGAGCAGGCTCCTGCACAGTGACTACATGAACATGACTCCCCGCCGCCCCGGGCCCACCCGCAAGCATTACCAG
CCCTATGCCCCACCACGCGACTTCGCAGCCTATCGCTCCAAACGGGGCAGAAAGAAACTCCTGTATATATTCAAA
CAACCATTTATGAGACCAGTACAAACTACTCAAGAGGAAGATGGCTGTAGCTGCCGATTTCCAGAAGAAGAAGAA
GGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCCTTTACCAGGGTCTCAGTACA
GCCACCAAGGACACCTACGACGCCCTTCACATGCAGGCCCTGCCCCCTCGCTAA
SEQ ID NO: 57. Nucleotide sequence of NT21_NT2-4h1_NYESO1-
TCRb28tmcytZa8tmBZh1_Xho-NY-ESO1 bmu2CD28tmcytCD3Zcyt-P2A-
1g4TCRa95LYCD8tmBBcytCD3Zcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTCGGGTCAAATTTTCACGCTCCGCTGATGCTCCTGCCTATCAACAAGGG
CAAAATCAATTGTACAATGAATTGAACTTGGGTAGAAGGGAAGAATATGACGTGCTCGATAAACGGAGGGGGAGA
GATCCAGAAATGGGCGGTAAACCACGGCGCAAAAATCCACAAGAGGGATTGTATAACGAGCTCCAAAAGGACAAA
ATGGCAGAAGCTTATTCAGAAATAGGAATGAAGGGGGAAAGGAGACGAGGTAAAGGTCATGACGGATTGTATCAA
GGATTGTCAACCGCTACTAAAGATACATATGATGCTTTGCATATGCAAGCTTTGCCTCCCAGAGCCAAGCGGTCT
GGGTCTGGGGCCACCAACTTCAGCCTGCTGAAGCAGGCCGGCGACGTGGAGGAGAACCCCGGCCCCATGGAGACC
CTCTTGGGCCTGCTTATCCTTTGGCTGCAGCTGCAATGGGTGAGCAGCAAACAGGAGGTGACGCAGATTCCTGCA
GCTCTGAGTGTCCCAGAAGGAGAAAACTTGGTTCTCAACTGCAGTTTCACTGATAGCGCTATTTACAACCTCCAG
TGGTTTAGGCAGGACCCTGGGAAAGGTCTCACATCTCTGTTGCTTATTCAGTCAAGTCAGAGAGAGCAAACAAGT
GGAAGACTTAATGCCTCGCTGGATAAATCATCAGGACGTAGTACTTTATACATTGCAGCTTCTCAGCCTGGTGAC
TCAGCCACCTACCTCTGTGCTGTGAGGCCCCTGTACGGAGGAAGCTACATACCTACATTTGGAAGAGGAACCAGC
CTTATTGTTCATCCGTATATCCAGAACCCTGACCCTGCCGTGTACCAGCTGAGAGACTCTAAATCCAGTGACAAG
TCTGTCTGCCTATTCACCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGAAAGTTCCGGC
TCCCCAAAATGTGATATCTACATCTGGGCGCCCTTGGCCGGGACTTGTGGGGTCCTTCTCCTGTCACTGGTTATC
ACCCTTTACTGCAAACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAGGACACCTACGACGCCCTT
CACATGCAGGCCCTGCCCCCTCGCTAA
SEQ ID NO: 58. Nucleotide sequence of NT22_NT2-4h2_NYESO1-
TCRb28tmcytZa8tmBh2_Xho-NY-ESO1 bmu2CD28tmcytCD3Zcyt-P2A-
1g4TCRa95LYCD8tmBBcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTCGGGTCAAATTTTCACGCTCCGCTGATGCTCCTGCCTATCAACAAGGG
CAAAATCAATTGTACAATGAATTGAACTTGGGTAGAAGGGAAGAATATGACGTGCTCGATAAACGGAGGGGGAGA
GATCCAGAAATGGGCGGTAAACCACGGCGCAAAAATCCACAAGAGGGATTGTATAACGAGCTCCAAAAGGACAAA
ATGGCAGAAGCTTATTCAGAAATAGGAATGAAGGGGGAAAGGAGACGAGGTAAAGGTCATGACGGATTGTATCAA
GGATTGTCAACCGCTACTAAAGATACATATGATGCTTTGCATATGCAAGCTTTGCCTCCCAGAGCCAAGCGGTCT
GGGTCTGGGGCCACCAACTTCAGCCTGCTGAAGCAGGCCGGCGACGTGGAGGAGAACCCCGGCCCCATGGAGACC
CTCTTGGGCCTGCTTATCCTTTGGCTGCAGCTGCAATGGGTGAGCAGCAAACAGGAGGTGACGCAGATTCCTGCA
GCTCTGAGTGTCCCAGAAGGAGAAAACTTGGTTCTCAACTGCAGTTTCACTGATAGCGCTATTTACAACCTCCAG
TGGTTTAGGCAGGACCCTGGGAAAGGTCTCACATCTCTGTTGCTTATTCAGTCAAGTCAGAGAGAGCAAACAAGT
GGAAGACTTAATGCCTCGCTGGATAAATCATCAGGACGTAGTACTTTATACATTGCAGCTTCTCAGCCTGGTGAC
TCAGCCACCTACCTCTGTGCTGTGAGGCCCCTGTACGGAGGAAGCTACATACCTACATTTGGAAGAGGAACCAGC
CTTATTGTTCATCCGTATATCCAGAACCCTGACCCTGCCGTGTACCAGCTGAGAGACTCTAAATCCAGTGACAAG
TCTGTCTGCCTATTCACCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGAAAGTTCCGGC
TCCCCAAAATGTGATATCTACATCTGGGCGCCCTTGGCCGGGACTTGTGGGGTCCTTCTCCTGTCACTGGTTATC
ACCCTTTACTGCAAACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACTGTAA
SEQ ID NO: 59. Nucleotide sequence of NT23_NT2-5h1_NYESO1-
TCRb28tmcytZa28tmBZhl_Xho-NY-ESO1 bmu2CD28tmcytCD3Zcyt-P2A-
1g4TCRa95LYCD28tmBBcytCD3Zcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTTCTGGGTTCTCGTCGTCGTGGGAGGT
GTGTTAGCATGTTACTCTCTCTTGGTTACTGTCGCTTTCATAATCTTTTGGGTCCGCTCAAAACGCTCTCGCTTG
TTACATTCCGATTATATGAATATGACACCTAGGAGACCTGGCCCGACTAGGAAACACTATCAACCTTACGCACCT
CCCAGAGATTTTGCTGCTTACAGGAGTCGGGTCAAATTTTCACGCTCCGCTGATGCTCCTGCCTATCAACAAGGG
CAAAATCAATTGTACAATGAATTGAACTTGGGTAGAAGGGAAGAATATGACGTGCTCGATAAACGGAGGGGGAGA
GATCCAGAAATGGGCGGTAAACCACGGCGCAAAAATCCACAAGAGGGATTGTATAACGAGCTCCAAAAGGACAAA
ATGGCAGAAGCTTATTCAGAAATAGGAATGAAGGGGGAAAGGAGACGAGGTAAAGGTCATGACGGATTGTATCAA
GGATTGTCAACCGCTACTAAAGATACATATGATGCTTTGCATATGCAAGCTTTGCCTCCCAGAGCCAAGCGGTCT
GGGTCTGGGGCCACCAACTTCAGCCTGCTGAAGCAGGCCGGCGACGTGGAGGAGAACCCCGGCCCCATGGAGACC
CTCTTGGGCCTGCTTATCCTTTGGCTGCAGCTGCAATGGGTGAGCAGCAAACAGGAGGTGACGCAGATTCCTGCA
GCTCTGAGTGTCCCAGAAGGAGAAAACTTGGTTCTCAACTGCAGTTTCACTGATAGCGCTATTTACAACCTCCAG
TGGTTTAGGCAGGACCCTGGGAAAGGTCTCACATCTCTGTTGCTTATTCAGTCAAGTCAGAGAGAGCAAACAAGT
GGAAGACTTAATGCCTCGCTGGATAAATCATCAGGACGTAGTACTTTATACATTGCAGCTTCTCAGCCTGGTGAC
TCAGCCACCTACCTCTGTGCTGTGAGGCCCCTGTACGGAGGAAGCTACATACCTACATTTGGAAGAGGAACCAGC
CTTATTGTTCATCCGTATATCCAGAACCCTGACCCTGCCGTGTACCAGCTGAGAGACTCTAAATCCAGTGACAAG
TCTGTCTGCCTATTCACCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTGGAGCAACAAATCTGAC
TTTGCATGTGCAAACGCCTTCAACAACAGCATTATTCCAGAAGACACCTTCTTCCCCAGCCCAGAAAGTTCCGGC
TCCCCAAAATTTTGGGTGCTGGTGGTGGTTGGTGGAGTCCTGGCTTGCTATAGCTTGCTAGTAACAGTGGCCTTT
ATTATTTTCTGGGTGAAACGGGGCAGAAAGAAACTCCTGTATATATTCAAACAACCATTTATGAGACCAGTACAA
ACTACTCAAGAGGAAGATGGCTGTAGCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTACAGCCACCAAGGACACCTACGACGCC
CTTCACATGCAGGCCCTGCCCCCTCGCTAA
SEQ ID NO: 60. Nucleotide sequence of NT24_NT2-4h3_aNY-TCR-CAR
b8tm28cytZa8tmBBcytZ_Xho-NY-ESO1 bmuCD8tmmu2CD28cytmuCD3Zcyt-
linkerP2A-1g4TCRa95LYCD8tmBBcytZcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTGCGACATTTATATTTGGGCCCCTCTC
GCTGGCACATGCGGCGTGTTGTTGCTCAGCCTCGTGATTACACTTTATTGTCGCTCAAAACGCTCTCGCTTGTTA
CATTCCGATTATATGAATATGACACCTAGGAGACCTGGCCCGACTAGGAAACACTATCAACCTTACGCACCTCCC
AGAGATTTTGCTGCTTACAGGAGTCGGGTCAAATTTTCACGCTCCGCTGATGCTCCTGCCTATCAACAAGGGCAA
AATCAATTGTACAATGAATTGAACTTGGGTAGAAGGGAAGAATATGACGTGCTCGATAAACGGAGGGGGAGAGAT
CCAGAAATGGGCGGTAAACCACGGCGCAAAAATCCACAAGAGGGATTGTATAACGAGCTCCAAAAGGACAAAATG
GCAGAAGCTTATTCAGAAATAGGAATGAAGGGGGAAAGGAGACGAGGTAAAGGTCATGACGGATTGTATCAAGGA
TTGTCAACCGCTACTAAAGATACATATGATGCTTTGCATATGCAAGCTTTGCCTCCCAGAGCCAAGCGGTCTGGG
TCTGGGGCCACCAACTTCAGCCTGCTGAAGCAGGCCGGCGACGTGGAGGAGAACCCCGGCCCCATGGAGACCCTC
TTGGGCCTGCTTATCCTTTGGCTGCAGCTGCAATGGGTGAGCAGCAAACAGGAGGTGACGCAGATTCCTGCAGCT
CTGAGTGTCCCAGAAGGAGAAAACTTGGTTCTCAACTGCAGTTTCACTGATAGCGCTATTTACAACCTCCAGTGG
TTTAGGCAGGACCCTGGGAAAGGTCTCACATCTCTGTTGCTTATTCAGTCAAGTCAGAGAGAGCAAACAAGTGGA
AGACTTAATGCCTCGCTGGATAAATCATCAGGACGTAGTACTTTATACATTGCAGCTTCTCAGCCTGGTGACTCA
GCCACCTACCTCTGTGCTGTGAGGCCCCTGTACGGAGGAAGCTACATACCTACATTTGGAAGAGGAACCAGCCTT
ATTGTTCATCCGTATATCCAGAACCCTGACCCTGCCGTGTACCAGCTGAGAGACTCTAAATCCAGTGACAAGTCT
GTCTGCCTATTCACCGATTTTGATTCTCAAACAAATGTGTCACAAAGTAAGGATTCTGATGTGTATATCACAGAC
AAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCGGCTCC
CCAAAATGTGATATCTACATCTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTATGAGACCAGTACAAACTACT
CAAGAGGAAGATGGCTGTAGCTGCCGATTTCCAGAAGAAGAAGAAGGAGGATGTGAACTGAGAGTGAAGTTCAGC
AGGAGCGCAGACGCCCCCGCGTACCAGCAGGGCCAGAACCAGCTCTATAACGAGCTCAATCTAGGACGAAGAGAG
GAGTACGATGTTTTGGACAAGAGACGTGGCCGGGACCCTGAGATGGGGGGAAAGCCGAGAAGGAAGAACCCTCAG
GAAGGCCTGTACAATGAACTGCAGAAAGATAAGATGGCGGAGGCCTACAGTGAGATTGGGATGAAAGGCGAGCGC
CGGAGGGGCAAGGGGCACGATGGCCTTTACCAGGGTCTCAGTACAGCCACCAAGGACACCTACGACGCCCTTCAC
ATGCAGGCCCTGCCCCCTCGCTAA
SEQ ID NO: 61. Nucleotide sequence of NT25_aNY-TCR-CAR b8tm28cytcyt-
a8tmBBcytcytZ_Xho-NY-ESO1 bmu2CD8tmCD28cytmu2CD28cytmu2CD3Zcyt-
linkerP2A-1g4TCRa95LYCD8tmmuBBcytBBcytZcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGTGCGACATTTATATTTGGGCCCCTCTC
GCTGGCACATGCGGCGTGTTGTTGCTCAGCCTCGTGATTACACTTTATTGTAGGAGTAAGAGGAGCAGGCTCCTG
CACAGTGACTACATGAACATGACTCCCCGCCGCCCCGGGCCCACCCGCAAGCATTACCAGCCCTATGCCCCACCA
CGCGACTTCGCAGCCTATCGCTCCCGCTCAAAACGCTCTCGCTTGTTACATTCCGATTATATGAATATGACACCT
AGGAGACCTGGCCCGACTAGGAAACACTATCAACCTTACGCACCTCCCAGAGATTTTGCTGCTTACAGGAGTCGG
GTCAAATTTTCACGCTCCGCTGATGCTCCTGCCTATCAACAAGGGCAAAATCAATTGTACAATGAATTGAACTTG
GGTAGAAGGGAAGAATATGACGTGCTCGATAAACGGAGGGGGAGAGATCCAGAAATGGGCGGTAAACCACGGCGC
AAAAATCCACAAGAGGGATTGTATAACGAGCTCCAAAAGGACAAAATGGCAGAAGCTTATTCAGAAATAGGAATG
AAGGGGGAAAGGAGACGAGGTAAAGGTCATGACGGATTGTATCAAGGATTGTCAACCGCTACTAAAGATACATAT
GATGCTTTGCATATGCAAGCTTTGCCTCCCAGAGCCAAGCGGTCTGGGTCTGGGGCCACCAACTTCAGCCTGCTG
AAGCAGGCCGGCGACGTGGAGGAGAACCCCGGCCCCATGGAGACCCTCTTGGGCCTGCTTATCCTTTGGCTGCAG
CTGCAATGGGTGAGCAGCAAACAGGAGGTGACGCAGATTCCTGCAGCTCTGAGTGTCCCAGAAGGAGAAAACTTG
GTTCTCAACTGCAGTTTCACTGATAGCGCTATTTACAACCTCCAGTGGTTTAGGCAGGACCCTGGGAAAGGTCTC
ACATCTCTGTTGCTTATTCAGTCAAGTCAGAGAGAGCAAACAAGTGGAAGACTTAATGCCTCGCTGGATAAATCA
TCAGGACGTAGTACTTTATACATTGCAGCTTCTCAGCCTGGTGACTCAGCCACCTACCTCTGTGCTGTGAGGCCC
CTGTACGGAGGAAGCTACATACCTACATTTGGAAGAGGAACCAGCCTTATTGTTCATCCGTATATCCAGAACCCT
GACCCTGCCGTGTACCAGCTGAGAGACTCTAAATCCAGTGACAAGTCTGTCTGCCTATTCACCGATTTTGATTCT
CAAACAAATGTGTCACAAAGTAAGGATTCTGATGTGTATATCACAGACAAAACTGTGCTAGACATGAGGTCTATG
GACTTCAAGAGCAACAGTGCTGTGGCCTGGAGCAACAAATCTGACTTTGCATGTGCAAACGCCTTCAACAACAGC
ATTATTCCAGAAGACACCTTCTTCCCCAGCCCAGAAAGTTCCGGCTCCCCAAAATGTGATATCTACATCTGGGCG
CCCTTGGCCGGGACTTGTGGGGTCCTTCTCCTGTCACTGGTTATCACCCTTTACTGCAAGAGAGGGCGTAAAAAG
CTGCTCTACATCTTTAAGCAGCCTTTCATGCGTCCTGTTCAGACAACACAGGAAGAGGACGGATGCTCTTGCAGG
TTCCCTGAGGAGGAGGAGGGTGGTTGCGAGCTCAAACGGGGCAGAAAGAAACTCCTGTATATATTCAAACAACCA
TTTATGAGACCAGTACAAACTACTCAAGAGGAAGATGGCTGTAGCTGCCGATTTCCAGAAGAAGAAGAAGGAGGA
TGTGAACTGAGAGTGAAGTTCAGCAGGAGCGCAGACGCCCCCGCGTACCAGCAGGGCCAGAACCAGCTCTATAAC
GAGCTCAATCTAGGACGAAGAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGCTAA
SEQ ID NO: 62. Nucleotide sequence of NT26_aNY-TCR-
CARb8h28pectmcytZa8h28pectmcytZ_Xho-NY-ESO1
bmu2CD8hmu2CD28pectmcytmu2Zcyt-P2A-1g4TCRa95LYCD8hCD28pectmcytZcyt-
Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGGCAAAACCGACGACCACCCCTGCCCCC
AGGCCTCCTACTCCCGCCCCGACGATTGCCAGCCAACCGTTAAGTTTAAGACCGGAAGCATGTAGACCGGCAGCT
GGTGGGGCTGTTCATACACGTGGCTTAGATTTTGCGCCTAGGAAGATCGAGGTAATGTACCCACCGCCCTATCTT
GATAACGAAAAATCTAACGGTACAATAATTCACGTCAAGGGCAAGCATTTGTGCCCTTCCCCGTTGTTCCCGGGC
CCAAGCAAACCGTTCTGGGTTCTCGTCGTCGTGGGAGGTGTGTTAGCATGTTACTCTCTCTTGGTTACTGTCGCT
TTCATAATCTTTTGGGTCCGCTCAAAACGCTCTCGCTTGTTACATTCCGATTATATGAATATGACACCTAGGAGA
CCTGGCCCGACTAGGAAACACTATCAACCTTACGCACCTCCCAGAGATTTTGCTGCTTACAGGAGTCGGGTCAAA
TTTTCACGCTCCGCTGATGCTCCTGCCTATCAACAAGGGCAAAATCAATTGTACAATGAATTGAACTTGGGTAGA
AGGGAAGAATATGACGTGCTCGATAAACGGAGGGGGAGAGATCCAGAAATGGGCGGTAAACCACGGCGCAAAAAT
CCACAAGAGGGATTGTATAACGAGCTCCAAAAGGACAAAATGGCAGAAGCTTATTCAGAAATAGGAATGAAGGGG
GAAAGGAGACGAGGTAAAGGTCATGACGGATTGTATCAAGGATTGTCAACCGCTACTAAAGATACATATGATGCT
TTGCATATGCAAGCTTTGCCTCCCAGAGCCAAGCGGTCTGGGTCTGGGGCCACCAACTTCAGCCTGCTGAAGCAG
GCCGGCGACGTGGAGGAGAACCCCGGCCCCATGGAGACCCTCTTGGGCCTGCTTATCCTTTGGCTGCAGCTGCAA
TGGGTGAGCAGCAAACAGGAGGTGACGCAGATTCCTGCAGCTCTGAGTGTCCCAGAAGGAGAAAACTTGGTTCTC
AACTGCAGTTTCACTGATAGCGCTATTTACAACCTCCAGTGGTTTAGGCAGGACCCTGGGAAAGGTCTCACATCT
CTGTTGCTTATTCAGTCAAGTCAGAGAGAGCAAACAAGTGGAAGACTTAATGCCTCGCTGGATAAATCATCAGGA
CGTAGTACTTTATACATTGCAGCTTCTCAGCCTGGTGACTCAGCCACCTACCTCTGTGCTGTGAGGCCCCTGTAC
GGAGGAAGCTACATACCTACATTTGGAAGAGGAACCAGCCTTATTGTTCATCCGTATATCCAGAACCCTGACCCT
GCCGTGTACCAGCTGAGAGACTCTAAATCCAGTGACAAGTCTGTCTGCCTATTCACCGATTTTGATTCTCAAACA
AATGTGTCACAAAGTAAGGATTCTGATGTGTATATCACAGACAAAACTGTGCTAGACATGAGGTCTATGGACTTC
AAGAGCAACAGTGCTGTGGCCTGGAGCAACAAATCTGACTTTGCATGTGCAAACGCCTTCAACAACAGCATTATT
CCAGAAGACACCTTCTTCCCCAGCCCAGAAAGTTCCGGCTCCCCAAAAGCTAAGCCCACCACGACGCCAGCGCCG
CGACCACCAACACCGGCGCCCACCATCGCGTCGCAGCCCCTGTCCCTGCGCCCAGAGGCGTGCCGGCCAGCGGCG
GGGGGCGCAGTGCACACGAGGGGGCTGGACTTCGCCCCTAGGAAAATTGAAGTTATGTATCCTCCTCCTTACCTA
GACAATGAGAAGAGCAATGGAACCATTATCCATGTGAAAGGGAAACACCTTTGTCCAAGTCCCCTATTTCCCGGA
CCTTCTAAGCCCTTTTGGGTGCTGGTGGTGGTTGGTGGAGTCCTGGCTTGCTATAGCTTGCTAGTAACAGTGGCC
TTTATTATTTTCTGGGTGAGGAGTAAGAGGAGCAGGCTCCTGCACAGTGACTACATGAACATGACTCCCCGCCGC
CCCGGGCCCACCCGCAAGCATTACCAGCCCTATGCCCCACCACGCGACTTCGCAGCCTATCGCTCCAGAGTGAAG
TTCAGCAGGAGCGCAGACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTACAGCCACCAAGGACACCTACGACGCC
CTTCACATGCAGGCCCTGCCCCCTCGCTAA
SEQ ID NO: 63. Nucleotide sequence of NT27_NT2b-4h1_aNY-TCR-CAR
bCD8h28pectmcytZa8tmBBcyt_Xho-NY-ESO1
bmu2CD8hmu2CD28pectmcytmu2Zcyt-P2A-1g4TCRa95LYCD8tmBBcyt-Not.xdna
ATGAGCATCGGCCTCCTGTGCTGTGCAGCCTTGTCTCTCCTGTGGGCAGGTCCAGTGAATGCTGGTGTCACTCAG
ACCCCAAAATTCCAGGTCCTGAAGACAGGACAGAGCATGACACTGCAGTGTGCCCAGGATATGAACCATGAATAC
ATGTCCTGGTATCGACAAGACCCAGGCATGGGGCTGAGGCTGATTCATTACTCAGTTGGTGCTGGTATCACTGAC
CAAGGAGAAGTCCCCAATGGCTACAATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAACACCGGGGAGCTGTTTTTTGGAGAA
GGCTCTAGGCTGACCGTACTGGAGGACCTGAAAAACGTGTTCCCACCCGAGGTCGCTGTGTTTGAGCCATCAGAA
GCAGAGATCTCCCACACCCAAAAGGCCACACTGGTGTGCCTGGCCACAGGCTTCTACCCCGACCACGTGGAGCTG
AGCTGGTGGGTGAATGGGAAGGAGGTGCACAGTGGGGTCAGCACAGACCCGCAGCCCCTCAAGGAGCAGCCCGCC
CTCAATGACTCCAGATACGCTCTGAGCAGCCGCCTGAGGGTCTCGGCCACCTTCTGGCAGGACCCCCGCAACCAC
TTCCGCTGTCAAGTCCAGTTCTACGGGCTCTCGGAGAATGACGAGTGGACCCAGGATAGGGCCAAACCCGTCACC
CAGATCGTCAGCGCCGAGGCCTGGGGTAGAGCAGACGGCTCTCCTAAGGCAAAACCGACGACCACCCCTGCCCCC
AGGCCTCCTACTCCCGCCCCGACGATTGCCAGCCAACCGTTAAGTTTAAGACCGGAAGCATGTAGACCGGCAGCT
GGTGGGGCTGTTCATACACGTGGCTTAGATTTTGCGCCTAGGAAGATCGAGGTAATGTACCCACCGCCCTATCTT
GATAACGAAAAATCTAACGGTACAATAATTCACGTCAAGGGCAAGCATTTGTGCCCTTCCCCGTTGTTCCCGGGC
CCAAGCAAACCGTTCTGGGTTCTCGTCGTCGTGGGAGGTGTGTTAGCATGTTACTCTCTCTTGGTTACTGTCGCT
TTCATAATCTTTTGGGTCCGCTCAAAACGCTCTCGCTTGTTACATTCCGATTATATGAATATGACACCTAGGAGA
CCTGGCCCGACTAGGAAACACTATCAACCTTACGCACCTCCCAGAGATTTTGCTGCTTACAGGAGTCGGGTCAAA
TTTTCACGCTCCGCTGATGCTCCTGCCTATCAACAAGGGCAAAATCAATTGTACAATGAATTGAACTTGGGTAGA
AGGGAAGAATATGACGTGCTCGATAAACGGAGGGGGAGAGATCCAGAAATGGGCGGTAAACCACGGCGCAAAAAT
CCACAAGAGGGATTGTATAACGAGCTCCAAAAGGACAAAATGGCAGAAGCTTATTCAGAAATAGGAATGAAGGGG
GAAAGGAGACGAGGTAAAGGTCATGACGGATTGTATCAAGGATTGTCAACCGCTACTAAAGATACATATGATGCT
TTGCATATGCAAGCTTTGCCTCCCAGAGCCAAGCGGTCTGGGTCTGGGGCCACCAACTTCAGCCTGCTGAAGCAG
GCCGGCGACGTGGAGGAGAACCCCGGCCCCATGGAGACCCTCTTGGGCCTGCTTATCCTTTGGCTGCAGCTGCAA
TGGGTGAGCAGCAAACAGGAGGTGACGCAGATTCCTGCAGCTCTGAGTGTCCCAGAAGGAGAAAACTTGGTTCTC
AACTGCAGTTTCACTGATAGCGCTATTTACAACCTCCAGTGGTTTAGGCAGGACCCTGGGAAAGGTCTCACATCT
CTGTTGCTTATTCAGTCAAGTCAGAGAGAGCAAACAAGTGGAAGACTTAATGCCTCGCTGGATAAATCATCAGGA
CGTAGTACTTTATACATTGCAGCTTCTCAGCCTGGTGACTCAGCCACCTACCTCTGTGCTGTGAGGCCCCTGTAC
GGAGGAAGCTACATACCTACATTTGGAAGAGGAACCAGCCTTATTGTTCATCCGTATATCCAGAACCCTGACCCT
GCCGTGTACCAGCTGAGAGACTCTAAATCCAGTGACAAGTCTGTCTGCCTATTCACCGATTTTGATTCTCAAACA
AATGTGTCACAAAGTAAGGATTCTGATGTGTATATCACAGACAAAACTGTGCTAGACATGAGGTCTATGGACTTC
AAGAGCAACAGTGCTGTGGCCTGGAGCAACAAATCTGACTTTGCATGTGCAAACGCCTTCAACAACAGCATTATT
CCAGAAGACACCTTCTTCCCCAGCCCAGAAAGTTCCGGCTCCCCAAAATGTGATATCTACATCTGGGCGCCCTTG
GCCGGGACTTGTGGGGTCCTTCTCCTGTCACTGGTTATCACCCTTTACTGCAAACGGGGCAGAAAGAAACTCCTG
TATATATTCAAACAACCATTTATGAGACCAGTACAAACTACTCAAGAGGAAGATGGCTGTAGCTGCCGATTTCCA
GAAGAAGAAGAAGGAGGATGTGAACTGTAA

### Compositions and Kits

In one aspect, this disclosure provides a composition comprising a plurality of genetically modified lymphocytes expressing chimeric antigen receptors or the chains thereof as described above for modulating the immune system of a subject.

Various lymphocytes can be used in this invention. Examples of Lymphocytes may include T cells, B cells, NK cells, macrophages, neutrophils, dendritic cells, mast cells, eosinophils, and basophils. In some embodiments, lymphocytes are derived from CD34 hematopoietic stem cells, embryonic stem cells, or induced pluripotent stem cells. Lymphocytes can be autologous, allogeneic, syngeneic, or xenogeneic. In some embodiments, lymphocytes are autologous. In some embodiments, lymphocytes are human lymphocytes.

In some embodiments, lymphocytes are peripheral blood lymphocytes (PBLs). In some embodiments, the lymphocytes can be tumor-infiltrating lymphocytes (TILs). In some embodiments, the lymphocytes may express a chimer antigen receptor. In some embodiments, the lymphocytes may express a recombinant T cell receptor. The CAR or TCR may bind to a cancer antigen of interest.

Examples of such cancer and/or tumor antigens include, but are not limited to, e.g., cyclin-dependent kinase-4, β-catenin, Caspase-8, MAGE-1, MAGE-3, Tyrosinase, Surface Ig idiogype, Her-2/neu, MUC-1, HPV E6, HPV E7, CD5, idiotype, CAMPATH-1, CD20, CEA, mucin-1, Lewis^{x}, CA-125, EGFR, p185^{HER2}, IL-2R, FAP, Tenascin, metalloproteinases, phCG, gp100 or Pmell7, HER2/neu, CEA, gp100, MARTI, TRP-2, melan-A, NY-ESO-1, MN (gp250), idiotype, MAGE-1, MAGE-3, Tyrosinase, Telomerase, MUC-1 antigens, and germ cell-derived tumor antigens, the blood group antigens, for example, Lea, Leb, LeX, LeY, H-2, B-1, B-2 antigens.

In certain embodiments, more than one cancer and/or tumor antigens can be bound by the same CAR-expressing T cell; for example, binding of one CAR of a T cell to a MAGE antigen can be combined with binding of another CAR of the T cell to another antigen, such as melanin A, tyrosinase, or gp100. For example, CD20 is a pan B antigen that is found on the surface of both malignant and non-malignant B cells that has proved to be an extremely effective target for immunotherapeutic antibodies for the treatment of non-Hodgkin's lymphoma. In this respect, pan T cell antigens such as CD2, CD3, CD5, CD6 and CD7 also comprise tumor-associated antigens within the meaning of the present invention. Still other exemplary tumor-associated antigens comprise, but are not limited to, MAGE-1, MAGE-3, MUC-1, HPV 16, HPV E6 & E7, TAG-72, CEA, L6-Antigen, CD19, CD22, CD37, CD52, HLA-DR, EGF Receptor and HER2 Receptor. In many cases immunoreactive antibodies (and/or immunoreactive antigen-binding fragments) for each of these antigens have been reported in the literature.

In embodiments, the two chains of the chimer antigen receptor are encoded by one nucleic acid transgene. The two chains can be linked by a self-cleaving peptide sequence. Alternatively, two sequences encoding the two chains are linked via a nucleic acid sequence comprising an IRES so that the two chains can be translated separately. The transgene expression can be regulated by a constitutively activated promoter or by an inducible promoter. In some embodiments, the transgene expression can be induced by the activation status of the lymphocyte. In others, the transgene can be introduced to the lymphocytes via integration-competent gamma-retroviruses or lentivirus, DNA transposition, etc.

The above-described genetically modified lymphocytes can be incorporated into pharmaceutical compositions suitable for administration. The pharmaceutical compositions generally comprise substantially isolated/purified lymphocytes and a pharmaceutically acceptable carrier in a form suitable for administration to a subject. Pharmaceutically acceptable carriers can be determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

The terms “pharmaceutically acceptable” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. In some embodiments, the composition includes the genetically modified lymphocytes as described above and optionally a cryo-protectant (e.g., glycerol, DMSO, PEG).

The composition or the pharmaceutical composition described herein can be provided in a kit. In one embodiment, the kit includes (a) a container that contains the composition and optionally (b) informational material. The informational material can be descriptive, instructional, marketing or other material that relates to the methods described herein and/or the use of the agents for therapeutic benefit. For example, kits may include instruction for the manufacturing, for the therapeutic regimen to be used, and periods of administration. In an embodiment, the kit includes also includes an additional therapeutic agent (e.g., a checkpoint modulator). The kit may comprise one or more containers, each with a different reagent. For example, the kit includes a first container that contains the composition and a second container for the additional therapeutic agent.

The containers can include a unit dosage of the pharmaceutical composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device. The device can be provided pre-loaded with one or both of the agents or can be empty, but suitable for loading.

### Methods for Preparing Compositions

In general, the practice of the present invention employs, unless otherwise indicated, conventional techniques of chemistry, molecular biology, recombinant DNA technology, PCR technology, immunology (e.g., antibody technology), expression systems (e.g., cell-free expression, phage display, ribosome display, and PROFUSION), and any necessary cell culture that are within the skill of the art and are explained in the literature. Although certain aspects of the present invention relate to compositions and uses of recombinant RNA retrovirus (e.g., lentiviral HIV-2, SIV, etc.), the molecular cloning may be done using proviral DNA clones, thus allowing the use of standard cloning techniques. Site-directed mutagenesis in vitro by synthetic oligodeoxynucleotides can be carried out according to methods known in the art. Genetic fusions, especially of use in the synthesis of fusion proteins, e.g., CARs, of the present invention can be made by art-recognized methods, e.g., gene SOE (splicing by overlap extension) methods that commonly rely upon the use of fusion primers (which are optionally mutagenic) during PCR amplification. (Horton et al. 1989 Gene 77:61-68; U.S. Pat. No. 5,023,171). Enzymatic amplification of DNA fragments by PCR technique can be carried out using a DNA thermal cycler according to manufacturer specifications.

Verification of the nucleotide sequences can be carried out by sequencing. Verification of whether a homologous recombination event has occurred between two homologous polypeptides that were and likely still are contained within a single vector of the present invention may be performed by any art-recognized method, including but not limited to, Northern blot and/or RT-PCR methods (e.g., if assessed directly within isolated retroviral genomes), Southern blot and/or PCR methods (e.g., if assessed upon host cell genomic DNAs comprising integrated retroviral vectors), and SDS-PAGE followed by Western blot and/or immunoprecipitation followed by SDS-PAGE and detection of labeled polypeptides (e.g., if homologous polypeptides are of discernible sizes and/or contain distinguishable domains, features and/or epitopes).

Some embodiments involve creating a retroviral vector encoding two or more identical or highly homologous molecules with degenerated codons. Methods for creating a retroviral vector encoding two or more identical or highly homologous molecules with degenerated codons employing silent mutation approach to reduce possible DNA recombination event has been previously described in e.g., U.S. Pat. No. 9,206,440 and Im E J et al., Recombination-deletion between homologous cassettes in retrovirus is suppressed via a strategy of degenerate codon substitution. Molecular Therapy—Methods & Clinical Development (2014) Article number: 14022). The references are incorporated by reference.

In another aspect, this disclosure provides a method of preparing the above-described composition. The method comprises: (a) providing a plurality of lymphocytes; (b) introducing to the plurality of lymphocytes a nucleic acid molecule encoding the first and the second polypeptide chains to obtain a plurality of genetically engineered lymphocytes; and (c) expanding the plurality of genetically engineered in a cell culture medium.

In some embodiments, the method may include: (a) providing a plurality of lymphocytes; (b) introducing to the plurality of lymphocytes a first nucleic acid molecule and a second nucleic acid molecule encoding the first and the second polypeptide chains, respectively, thereby obtaining a plurality of genetically-engineered lymphocytes; and (c) expanding the plurality of genetically-engineered in a cell culture medium. In some embodiments, the method may additionally include expanding a first plurality of lymphocytes in a cell culture medium following the step of introducing the first nucleic acid or expanding a second plurality of lymphocytes in a cell culture medium following the step of introducing the second nucleic acid.

Methods to obtain a composition of tumor-specific genetically modified subsets of lymphocytes described above can be performed in vitro or ex vivo. Methods in more particular form may be as disclosed in PCT/EP2018/080343, the content of which is hereby incorporated by reference in its entirety.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

### Lymphocytes

Prior to the expansion and genetic modification of the lymphocytes, a source of lymphocytes from a subject is obtained. Lymphocytes can be obtained from several sources, including peripheral blood mononuclear cells, bone marrow, lymph node tissue, umbilical cord blood, thymus tissue, tissue from an infection site, ascites, pleural effusion, splenic tissue, and tumors. As described herein, any number of lymphocyte lines available in the art can be used. Lymphocytes can be obtained from a unit of blood collected from a subject using any number of techniques known to the person skilled in the art, such as the Ficoll™ separation. Circulating blood cells of an individual can be obtained by apheresis. The apheresis product typically contains lymphocytes, including T lymphocytes, monocytes, granulocytes, B lymphocytes, other nucleated white blood cells, red blood cells, and platelets. The cells harvested by apheresis can be washed to remove the plasma fraction and place the cells in a suitable buffer or medium for the subsequent processing steps. The cells may be washed with PBS. Alternatively, the wash solution may lack calcium and may lack magnesium or may lack many, if not all, divalent cations. As those of ordinary skill in the art would readily appreciate, a washing step can be achieved by methods known to those skilled in the art, such as using a semiautomatic continuous flow centrifuge (e.g., the Cobe 2991 cell processor, the Baxter CytoMate, or e1Haemonetics Cell Saver 5) according to the manufacturer's instructions. After washing, the cells can be resuspended in a variety of biocompatible buffers, such as, for example, Ca2+ free, PBS free Mg2+, PlasmaLyte A, or other saline solution with or without buffer. Alternatively, the undesirable components of the apheresis sample can be removed and the cells resuspended directly in a culture medium.

As described herein, lymphocytes may be isolated from peripheral blood by lysis of red blood cells and depletion of monocytes, for example, by centrifugation through a PERCOLL gradient or by countercurrent centrifugal elutriation. If needed, specific subpopulation lymphocytes, such as T lymphocytes (i.e., Cd3+, CD28+, CD4+, CD8+, CD45RA+ or CD45RO+T lymphocytes) can be further isolated by positive or negative selection techniques. For example, T lymphocytes may be isolated by incubation with conjugated anti-CD3/anti-CD28 beads (i.e., 3×28), such as DYNABEADS M-450 CD3/CD28 T, for a sufficient period of time (i.e., 30 minutes to 24 hours) for positive selection of the desired T lymphocytes. For isolation of T lymphocytes from patients with leukemia, the use of longer incubation times, such as 24 hours, can increase cellular performance. Longer incubation times can be used to isolate T lymphocytes in any situation where there are few T lymphocytes compared to other cell types, such as isolating TILs from tumor tissue or from immunocompromised individuals. The person skilled in the art will recognize that multiple rounds of selection may also be used. It may be desirable to perform the selection procedure and use the “unselected” cells in the activation and expansion process. “Unselected” cells can also undergo new rounds of selection.

Enrichment of a population of lymphocytes (e.g., T lymphocytes) by negative selection can be performed with a combination of antibodies directed to unique surface markers for the negatively selected cells. One method is the sorting and/or selection of cells by negative magnetic immune adherence or flow cytometry using a cocktail of monoclonal antibodies directed to cell surface markers present in the negatively selected cells. For example, to enrich CD4+ cells by negative selection, a monoclonal antibody typically includes antibodies against CD14, CD20, CD11b, CD16, HLA-DR, and CD8. Alternatively, the regulatory T lymphocytes are depleted by anti-C25 conjugate beads or other similar selection method.

Lymphocytes for stimulation can also be frozen after a washing step. Wishing not to be bound by theory, freezing and the following thawing step provide a more uniform product by eliminating granulocytes and, to some extent, monocytes in the cell population. After the washing step that removes the plasma and platelets, the cells can be suspended in a freezing solution. Although many solutions and freezing parameters are known in the art and will be useful in this context, one method involves the use of PBS containing 20% DMSO and 8% human serum albumin, or culture medium containing 10% dextran 40 and 5% dextrose human albumin and 7.5% DMSO or 31.25% Plasmalyte A, 31.25% dextrose 5%, 0.45% NaCl, 10% dextran 40 and 5% of dextrose, 20% serum of human albumin and 7.5% of DMSO or other suitable cell freezing medium containing for example Hespan and PlasmaLyte A. The cells may then be frozen at −80° C. at a rate of 1° C. per minute and stored in the vapor phase of a liquid nitrogen storage tank. Other methods of controlled freezing can be used, as well as uncontrolled freezing immediately at −20° C. or in liquid nitrogen.

The cryopreserved cells may be thawed and washed as described herein and allowed to stand for one hour at room temperature before activation using the methods of the present invention. As described herein, lymphocytes can be expanded, frozen, and used later. As described herein, samples may be collected from a patient shortly after the diagnosis of a particular disease as described herein, but before any treatment. The cells may be isolated from a blood sample or an apheresis of a subject before any number of relevant treatment modalities, including but not limited to treatment with agents such as natalizumab, efalizumab, antiviral agents, chemotherapy, radiation, immunosuppressive agents such as cyclosporine, azathioprine, methotrexate, mycophenolate and FK506, antibodies or other immunoablatories such as CAMPATH, anti-CD3 antibodies, cytoxane, fludarabine, cyclosporin, FK506, rapamycin, mycophenolic acid, steroids, FR901228, and irradiation. These drugs inhibit calcium-dependent calcineurin phosphatase (e.g., ciclosporin and FK506) or inhibit p70S6 kinase that is important for signaling induced by the growth factor (rapamycin) (Liu et al., Cell 66:807-815, 1991; Henderson et al., Immun 73:316-321, 1991, Bierer et al., Curr. Opin. Immun., 5:763-773, 1993). The cells may be isolated from a patient and frozen for later use together with (e.g., before, simultaneously or after) bone marrow or stem cell transplant, therapy with T lymphocyte ablation using chemotherapeutic agents such as fludarabine, radiotherapy external beam (XRT), cyclophosphamide, or antibodies such as OKT3 or CAMPATH. As described herein, the cells may be isolated before and can be frozen for later use in the treatment after therapy with ablation of B lymphocytes, such as agents that react with CD20, for example, Rituxan.

Either before or after the genetic modification of lymphocytes to express a desirable transgene, lymphocytes can be activated and expanded generally using methods such as those described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. patent application No. 20060121005.

### Vectors

Transgenes can be introduced into lymphoid cells using various methods. These methods include, but are not limited to, transduction of cells using integration-competent gamma-retroviruses or lentivirus, and DNA transposition.

A wide variety of vectors can be used for the expression of the transgenes. The ability of certain viruses to infect cells or enter cells via receptor-mediated endocytosis, and to integrate into a host cell genome and express viral genes stably and efficiently have made them attractive candidates for the transfer of foreign nucleic acids into cells. Accordingly, in certain embodiments, a viral vector is used to introduce a nucleotide sequence encoding one or more transgenes or fragment thereof into a host cell for expression. The viral vector may comprise a nucleotide sequence encoding one or more transgenes or fragment thereof operably linked to one or more control sequences, for example, a promoter. Alternatively, the viral vector may not contain a control sequence and will instead rely on a control sequence within the host cell to drive expression of the transgenes or fragment thereof. Non-limiting examples of viral vectors that may be used to deliver a nucleic acid include adenoviral vectors, AAV vectors, and retroviral vectors.

For example, an adeno-associated virus (AAV) can be used to introduce a nucleotide sequence encoding one or more transgenes or fragment thereof into a host cell for expression. AAV systems have been described previously and are generally well known in the art (Kelleher and Vos, Biotechniques, 17 (6): 1110-7, 1994; Cotten et al., Proc Natl Acad Sci USA, 89 (13): 6094-6098, 1992; Curiel, Nat Immun, 13 (2-3): 141-64, 1994; Muzyczka, Curr Top Microbiol Immunol, 158:97-129, 1992). Details concerning the generation and use of rAAV vectors are described, for example, in U.S. Pat. Nos. 5,139,941 and 4,797,368, each incorporated herein by reference in its entirety for all purposes.

In some embodiments, a retroviral expression vector can be used to introduce a nucleotide sequence encoding one or more transgenes or fragment thereof into a host cell for expression. These systems have been described previously and are generally well known in the art (Nicolas and Rubinstein, In, Rodriguez and Denhardt, eds., Stoneham: Butterworth, pp. 494-513, 1988; Temin, In: Gene Transfer, Kucherlapati (ed.), New York: Plenum Press, pp. 149-188, 1986). Examples of vectors for eukaryotic expression in mammalian cells include AD5, pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

Combinations of retroviruses and an appropriate packaging line may also find use, where the capsid proteins will be functional for infecting the target cells. Usually, the cells and viruses will be incubated for at least about 24 hours in the culture medium. The cells are then allowed to grow in the culture medium for short intervals in some applications, e.g., 24-73 hours, or for at least two weeks, and may be allowed to grow for five weeks or more, before analysis. Commonly used retroviral vectors are “defective,” i.e., unable to produce viral proteins required for productive infection. Replication of the vector requires growth in the packaging cell line. The host cell specificity of the retrovirus is determined by the envelope protein, env (pl20). The envelope protein is provided by the packaging cell line. Envelope proteins are of at least three types, ecotropic, amphotropic and xenotropic. Retroviruses packaged with ecotropic envelope protein, e.g., MMLV, are capable of infecting most murine and rat cell types. Ecotropic packaging cell lines include BOSC23. Retroviruses bearing amphotropic envelope protein, e.g., 4070A, are capable of infecting most mammalian cell types, including human, dog, and mouse. Amphotropic packaging cell lines include PA12 and PA317. Retroviruses packaged with xenotropic envelope protein, e.g., AKR env, are capable of infecting most mammalian cell types, except murine cells. The vectors may include genes that must later be removed, e.g., using a recombinase system such as Cre/Lox, or the cells that express them destroyed, e.g., by including genes that allow selective toxicity such as herpesvirus TK, BCL-xs, etc. Suitable inducible promoters are activated in a desired target cell type, either the transfected cell or progeny thereof.

Non-limiting examples of the vectors useful include retroviral vector SFG.MCS, and helper plasmids RD114, Peg-Pam3 (Arber et al. J Clin Invest 2015 Jan. 2; 125 (1): 157-168), lentiviral vector pRRL, and helper plasmids R8.74 and pMD2G (e.g., Addgene Plasmid #12259). In some embodiments, the Sleeping Beauty transposon system can be used (Deniger et al. 2016 Mol Ther. June; 24 (6): 1078-1089). In some embodiments, transgenes can be introduced into cells via deforming a cell as it passes through a small opening, disrupting the cell membrane and allowing material to be inserted into the cell, for example, electroporation (Xiaojun et al. 2017 Protein Cell, 8 (7): 514-526), or the Cell Squeeze® method. Such electroporation methods of an RNA encoding a transgene allow for transient expression of such transgene in cells which can limit toxicity and other undesirable effects of engineered cells (Barrett et al. 2011 Hum Gene Ther. December; 22 (12): 1575-1586).

Physical methods for introducing a polynucleotide into a host cell include calcium phosphate precipitation, lipofection, particle bombardment, microinjection, electroporation, and the like. Methods for producing cells comprising exogenous vectors and/or nucleic acids are well known in the art. See, for example, Sambrook et al. (2001, Molecular Cloning: A Laboratory Manual, Cold Spring Harbor Laboratory, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as an in vitro and in vivo release vehicle is a liposome (e.g., an artificial membrane vesicle).

In the case where a non-viral delivery system is used, an exemplary delivery vehicle is a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, bound to a liposome via a binding molecule that is associated with both the liposome and the oligonucleotide, entrapped in a liposome, in a complex with a liposome, dispersed in a solution containing a lipid, mixed with a lipid, combined with a lipid, contained as a suspension in a lipid, content or in a complex with a micelle, or associated otherwise with a lipid. The compositions associated with lipids, lipids/DNA or lipids/expression vector are not limited to any particular structure in solution. For example, they can be present in a bilayer structure, as micelles, or with a “collapsed” structure. They can also be simply interspersed in a solution, possibly forming aggregates that are not uniform in size or shape. Lipids are fatty substances that can be natural or synthetic lipids. For example, lipids include fatty droplets that occur naturally in the cytoplasm as well as the class of compounds containing long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; Dicetylphosphate (“DCP”) can be obtained from K & K Laboratories (Plainview, NY); Cholesterol (“Choi”) can be obtained from Calbiochem-Behring; dimyristyl phosphatidylglycerol (“DMPG”) and other lipids can be obtained from Avanti Polar Lipids, Inc. (Birmingham, AL). Lipid stock solutions in chloroform or chloroform/methanol can be stored at about −20° C. Chloroform is used as the sole solvent since it evaporates more easily than methanol. “Liposome” is a generic term that encompasses a variety of unique and multilamellar lipid vehicles formed by the generation of bilayers or closed lipid aggregates. Liposomes can be characterized as having vesicular structures with a bilayer membrane of phospholipids and an internal aqueous medium. Multilamellar liposomes have multiple layers of lipids separated by an aqueous medium. They form spontaneously when phospholipids are suspended in an excess of aqueous solution. The lipid components undergo self-rearrangement before the formation of closed structures and trap dissolved water and solutes between the lipid bilayers (Ghosh et al., 1991 Glycobiology 5:505-10). However, compositions that have different structures in solution than the normal vesicular structure are also included. For example, lipids can assume a micellar structure or simply exist as nonuniform aggregates of lipid molecules. Lipofectamine-nucleic acid complexes are also contemplated.

Regardless of the method used to introduce exogenous nucleic acids into a host cell, the presence of the recombinant DNA sequence in the host cell can be confirmed by a series of tests. Such assays include, for example, “molecular biology” assays well known to those skilled in the art, such as Southern and Northern blot, RT-PCR and PCR; biochemical assays, such as the detection of the presence or absence of a particular peptide, for example, by immunological means (ELISA and Western blot) or by assays described herein to identify agents that are within the scope of the invention.

### Methods of Treatment

The agents described above (e.g., vectors and cells) can be used as immunotherapeutics in treatment of various disease. Accordingly, this disclosure further provides a method of a disorder, such as infection, cancer or a tumor. The method comprises administering a therapeutically effective amount of the composition or the pharmaceutical composition, as described above, to a subject in need thereof.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc) and a human). The subject may be a human or a non-human. In more exemplary aspects, the mammal is a human. In some embodiments, the subject is a human. In some embodiments, the subject has a cancer. In some embodiments, the subject is immune-depleted.

### Cancer

In some embodiments, the agents described above (e.g., vectors and cells) can be used as immunotherapeutics in treating cancer or a tumor. As used herein, “cancer,” “tumor,” and “malignancy” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods of the present invention may be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., leukemias and lymphomas) or may comprise solid tumors. The types of cancers to be treated with the compositions of the present invention include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

Hematologic cancers are cancers of the blood or bone marrow. Examples of hematologic (or haematogenous) cancers include leukemias, including acute leukemias (such as acute lymphocytic leukemia, acute myelocytic leukemia, acute myelogenous myelogenous leukemia, promyelocytic, myelomonocytic, monocytic and erythroleukemia), chronic leukemias (such as chronic myelocytic (granulocytic) leukemia, chronic myelogenous leukemia and chronic lymphocytic leukemia), polycythemia vera, lymphoma, Hodgkin's disease, non-Hodgkin's lymphoma (indolent and high-grade forms), myeloma Multiple, Waldenstrom's macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

The agents described above can also be used as immunotherapeutics in treatment of infectious disease; for example, in procedures that employ CARs that recognize infectious disease antigens. Accordingly, polypeptides described herein can be made that bind to any of a number of forms of infectious disease antigen, thereby inducing an immune response to the infectious disease antigen upon binding. Infectious disease antigens to which CARs described herein can be designed to bind include, but are not limited to, bacterial antigens, viral antigens, fungal antigens, parasitic antigens, and microbial toxins. Exemplary forms of each class of antigen are considered in greater detail below.

### Bacteria

Examples of bacteria (specifically, epitopes thereof) to which polypeptides or CARs of the instant invention may bind include, but are not limited to: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella enterica Typhimurium, Salmonella enterica Typhi, Salmonella enterica Paratyphi, Salmonella enterica Enteridtidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus. In a particular embodiment, a construct of the invention comprises a binding molecule which binds to Staphylococcal protein A.

### Viruses

Examples of viruses (or epitopes thereof) which may be bound by polypeptides or CARs of the instant invention include, but are not limited to: polyomavirus JC (JCV), human immunodeficiency virus type I (HIV I), hepatitis B virus (HBV), hepatitis C virus (HCV), cytomegalovirus (CMV), Epstein Barr virus (EBV), influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), Streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651 693; Tiollais et al., 1985, Nature 317:489 495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type I glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, the bovine parainfluenza virus type 3 hemagglutinin neuraminidase), bovine viral diarrhea virus glycoprotein 48 or glycoprotein 53, hepatitis type A, influenza, varicella, adenovirus, herpes simplex type I (HSV I), herpes simplex type II (HSV II), rinderpest, rhinovirus, echovirus, rotavirus, respiratory syncytial virus, papilloma virus, papova virus, echinovirus, arbovirus, hantavirus, coxsackie virus, mumps virus, measles virus, rubella virus, polio virus, human immunodeficiency virus type II (HIV II), any picornaviridae, enteroviruses, caliciviridae, any of the Norwalk group of viruses, togaviruses, such as alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, human herpesvirus 6, cercopithecine herpes virus I (B virus), and poxviruses.

In certain embodiments, polypeptides or CARs of the instant invention bind to HIV, inducing an immune response to the virus in a subject to whom the viral vector is administered. Various antigenic domains (e.g., epitopes) of HIV are known in the art and such domains include structural domains such as Gag, Gag-polymerase, Gag-protease, reverse transcriptase (RT), integrase (IN) and Env. The structural domains of HIV are often further subdivided into polypeptides, for example, p55, p24, p6 (Gag); p160, p10, p15, p31, p65 (pol, prot, RT and IN); and gp160, gp120 and gp41 (Ems) or Ogp140 as constructed by Chiron Corporation. Molecular variants of such polypeptides can also be targeted for binding by the polypeptides or CARs of the instant invention, for example, variants such as those described in PCT/US99/31245, PCT/US99/31273 and PCT/US99/31272.

### Fungi

Examples of fungi (or epitopes thereof) which may be bound by polypeptides or CARs of the instant invention include, but are not limited to fungi from the genus Mucor, Candida, and Aspergillus, e.g., Mucor racmeosus, Candida albicans, and Aspergillus niger.

### Parasites

Examples of parasites (or epitopes thereof) which may be bound by polypeptides or CARs of the instant invention include, but are not limited to: Toxoplasma gondii, Treponema pallidun, Malaria, and Cryptosporidium.

### Microbial Toxins

Examples of microbial toxins (or epitopes thereof) which may be bound by polypeptides or CARs of the instant invention include, but are not limited to: toxins produced by Bacillus anthracis, Bacillus cereus, Bordatella pertussis, Clostridium botulinum, Clostridium perfringens, Clostridium tetani, Croynebacterium diptheriae, Salmonella sp. Shigella sp., Staphylococcus sp., and Vibrio cholerae. Toxins such as ricin from jack bean and other naturally occurring (e.g., produced by an organism) and man-made toxins or portions thereof may also be bound by the polypeptides or CARs of the instant invention.

The pharmaceutical compositions, as described, can be administered in a manner appropriate to the disease to be treated (or prevented). The amount and frequency of administration will be determined by factors such as the condition of the patient, and the type and severity of the patient's disease, although appropriate dosages can be determined by clinical trials.

When “an immunologically effective amount,” “an effective antitumor quantity,” “an effective tumor-inhibiting amount” or “therapeutic amount” is indicated, the precise amount of the compositions of the present invention to be administered can be determined by a physician having account for individual differences in age, weight, tumor size, extent of infection or metastasis, and patient's condition (subject). It can generally be stated that a pharmaceutical composition comprising the lymphocytes described herein can be administered at a dose of 10⁴ to 10⁹ cells/kg body weight, e.g., 10⁵ to 10⁶ cells/kg body weight, including all values integers within these intervals. The lymphocyte compositions can also be administered several times at these dosages. The cells can be administered using infusion techniques that are commonly known in immunotherapy (see, e.g., Rosenberg et al., New Eng. J. of Med. 319:1676, 1988). The optimal dose and treatment regimen for a particular patient can be readily determined by one skilled in the art of medicine by monitoring the patient for signs of the disease and adjusting the treatment accordingly.

The administration of the present compositions can be carried out in any convenient way, including infusion or injection (i.e., intravenous, intrathecal, intramuscular, intraluminal, intratracheal, intraperitoneal, or subcutaneous), transdermal administration, or other methods known in the art. Administration can be once every two weeks, once a week, or more often, but the frequency may be decreased during a maintenance phase of the disease or disorder. In some embodiments, the composition is administered by intravenous infusion.

In certain cases, the cells activated and expanded using the methods described herein, or other methods known in the art wherein the lymphocytes are expanded to therapeutic levels, are administered to a patient together with (e.g., before, simultaneously or after) any number of relevant treatment modalities. Also described herein, the lymphocytes can be used in combination with chemotherapy, radiation, immunosuppressive agents, such as cyclosporin, azathioprine, methotrexate, mycophenolate, and FK506, antibodies, or other immunoablating agents such as CAMPATH, anti-cancer antibodies. CD3 or other antibody therapies, cytoxine, fludarabine, cyclosporine, FK506, rapamycin, mycophenolic acid, steroids, FR901228, cytokines, and irradiation.

The compositions of the present invention can also be administered to a patient together with (e.g., before, simultaneously or after) bone marrow transplantation, therapy with T lymphocyte ablation using chemotherapy agents such as fludarabine, radiation therapy external beam (XRT), cyclophosphamide, or antibodies such as OKT3 or CAMPATH. Also described herein, the compositions can be administered after ablative therapy of B lymphocytes, such as agents that react with CD20, for example, Rituxan. For example, subjects may undergo standard treatment with high-dose chemotherapy followed by transplantation of peripheral blood stem cells. In certain cases, after transplantation, the subjects receive an infusion of the expanded lymphocytes, or the expanded lymphocytes are administered before or after surgery.

In some embodiments, the method may further include administering to the subject a second therapeutic agent. The second therapeutic agent is an anti-cancer or anti-tumor agent. In some embodiments, the composition is administered to the subject before, after, or concurrently with the second therapeutic agent, including chemotherapeutic agents and immunotherapeutic agents.

In some embodiments, the method further comprises administering a therapeutically effective amount of an immune checkpoint modulator. Examples of the immune checkpoint modulator may include PD1, PDL1, CTLA4, TIM3, LAG3, and TRAIL. The checkpoint modulators may be administered simultaneously, separately, or concurrently with the composition of the present invention.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, methyldopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, see, e.g., Agnew Chem. Intl. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®) and doxetaxel (TAXOTERE®); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, xeloda, gemcitabine, KRAS mutation covalent inhibitors and goserelin; and pharmaceutically acceptable salts, acids or derivatives of any of the above. Additional examples include irinotecan, oxaliplatinum, and other standard colon cancer regimens.

An “immunotherapeutic agent” is a biological agent useful in the treatment of cancer. Examples of immunotherapeutic agents include atezolizumab, avelumab, blinatumomab, daratumumab, cemiplimab, durvalumab, elotuzumab, laherparepvec, ipilimumab, nivolumab, obinutuzumab, ofatumumab, pembrolizumab, cetuximab, and talimogene.

In the examples below, one approach described above was used for enhancing adoptive immunotherapy efficacy for cancer that expresses tumor-associated antigen NY-ESO-1 in the context of HLA-A0201 through engineering T cells with a TCR-based chimeric antigen receptor that holds high affinity and specificity to NY-ESO-1 in the context of HLA-A0201.

NY-ESO-1 is a protein normally expressed only in fetal and testicular tissue, but aberrantly expressed by some solid malignancies. This added NY-ESO-1 to a growing list of molecules expressed appropriately in the germ line and abnormally by some cancers. These molecules were termed “cancer/testis” antigens, and are capable of serving as targets for antigen-directed immunotherapies. The cancer-testis antigen NY-ESO-1 is expressed by many solid tumors and has limited expression by mature somatic tissues, making it a highly attractive target for tumor immunotherapy. Targeting NY-ESO-1 using engineered T cells has demonstrated clinical efficacy in the treatment of some adult tumors.

The approach involves: (1) integrating TCR co-stimulation signaling element in the CAR design; (2) creating a vector nucleic acid sequence that comprises two or more nucleic acid sequences that encode identical polypeptide sequences, by silently mutating one of the exogenous nucleic acid sequences using degenerate codons for the purpose of reducing the homology between the two nucleic acid sequences while maintaining the encoded polypeptide sequence; (3) integrating repeat units of TCR co-stimulation signaling elements of human CD28, or human 4-1BB or a combination of the two; and (4) comprising a same or different transmembrane domains in a same TCR-CAR construct. Specific nucleic acid sequences of such CAR genes are also disclosed. This approach created TCR-based CARs with the anti-NY-ESO-1 specificity by employing the EC domains of the alpha chain and beta chain of the 1G4 195LY TCR and incorporating TCR signaling element CD3Z or CD3E together with co-stimulating element CD28, or 4-1BB, or a combination of CD28 and 4-1BB in order to enhance TCR-CAR expressing T cell activation via TCR-CAR-mediated stimulation. This leads to increased anti-tumor activity in patients.

### Definitions

A T-cell receptor or TCR is a protein complex found on the surface of T cells, or T lymphocytes, that is responsible for recognizing fragments of antigen as peptides bound to MHC molecules. The binding between TCR and antigen peptides is of relatively low affinity and is degenerate: that is, many TCRs recognize the same antigen peptide and many antigen peptides are recognized by the same TCR. A majority of T cells have a TCR existing as a complex of several proteins. A TCR is composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRI), respectively). This ratio changes during ontogeny and in diseased states (such as leukemia). It also differs between species. Each chain of a TCR is composed of two extracellular domains: variable (V) region and a constant (C) region. The constant region is proximal to the cell membrane, followed by a transmembrane region and a short cytoplasmic tail, while the variable region binds to the peptide/MHC complex. For the purpose of the present invention, the term “constant region of a T cell receptor chain or a portion thereof” also includes embodiments wherein the constant region of a T cell receptor chain is (from N terminus to C terminus) followed by a transmembrane region and a cytoplasmic tail, such as a transmembrane region and a cytoplasmic tail which are naturally linked to the constant region of a T cell receptor chain.

The term “antigen receptor” or “antigen recognizing receptor” as used herein refers to a receptor that is capable of activating an immune cell (e.g., a T-cell) in response to antigen binding. In particular, the term “antigen receptor” includes engineered receptors, which confer an arbitrary specificity onto an immune effector cell such as a T cell. An antigen receptor according to the invention may be present on T cells, e.g. instead of or in addition to the T cell's own T cell receptor. Such T cells do not necessarily require processing and presentation of an antigen for recognition of the target cell but rather may recognize preferably with specificity any antigen present on a target cell. Preferably, said antigen receptor is expressed on the surface of the cells. Specifically, the term includes artificial or recombinant receptors comprising a single molecule or a complex of molecules which recognize, i.e. bind to, a target structure (e.g. an antigen) on a target cell (e.g. by binding of an antigen binding site or antigen binding domain to an antigen expressed on the surface of the target cell) and may confer specificity onto an immune effector cell such as a T cell expressing said antigen receptor on the cell surface. Preferably, recognition of the target structure by an antigen receptor results in activation of an immune effector cell expressing said antigen receptor. An antigen receptor may comprise one or more protein units said protein units comprising one or more domains as described herein. The term “antigen receptor” preferably does not include naturally occurring T cell receptors. According to the invention, the term “antigen receptor” is preferably synonymous with the terms “chimeric antigen receptor”, “chimeric T cell receptor” and “artificial T cell receptor.”

Exemplary antigen recognizing receptors may be native or genetically engineered TCRs, or genetically engineered TCR-like mAbs (Hoydahl et al. Antibodies 2019 8:32) or CARs in which a tumor antigen-binding domain is fused to an intracellular signaling domain capable of activating an immune cell (e.g., a T-cell). T-cell clones expressing native TCRs against specific cancer antigens have been previously disclosed (Traversari et al, J Exp Med, 1992 176:1453-7; Ottaviani et al, Cancer Immunol Immunother, 2005 54:1214-20; Chaux et al, J Immunol, 1999 163:2928-36; Luiten and van der Bruggen, Tissue Antigens, 2000 55:149-52; van der Bruggen et al, Eur J Immunol, 1994 24:3038-43; Huang et al, J Immunol, 1999 162:6849-54; Ma et al, Int J Cancer, 2004 109:698-702; Ebert et al, Cancer Res, 2009 69:1046-54; Ayyoub et al J Immunol 2002 168:1717-22; Chaux et al, European Journal of Immunology, 2001 31:1910-16; Wang et al, Cancer Immunol Immunother, 2007 56:807-18; Schultz et al, Cancer Research, 2000 60:6272-75; Cesson et al, Cancer Immunol Immunother, 2010 60:23-25; Zhang et al, Journal of Immunology, 2003 171:219-25; Gnjatic et al, PNAS, 2003 100:8862-67; Chen et al, PNAS, 2004.

The term “Chimeric Antigen Receptor” or “CAR” refers to a set of polypeptides, typically two in the simplest embodiments, which when in an immune effector cell, provides the cell with specificity for a target cell and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen-binding domain to an intracellular signaling domain. In one aspect, the stimulatory molecule of the CAR is the zeta chain associated with the T cell receptor complex (e.g., CD3 zeta). In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27, and/or CD28.

The term “TCR-based CAR” or “TCR-CAR” refers to a CAR that comprises an antigen-binding domain formed by TCR α, β, γ, or δ chains or antigen-binding portions thereof.

The term “stimulatory molecule,” refers to a molecule expressed by an immune cell, (e.g., T cell, NK cell, or B cell) that provides the cytoplasmic signaling sequence(s) that regulate activation of the immune cell in a stimulatory way for at least some aspect of the immune cell-signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif, which is known as immunoreceptor tyrosine-based activation motif or ITAM. Examples of an ITAM containing cytoplasmic signaling sequence that is of particular use in the invention includes, but is not limited to, those derived from CD3 zeta, common FcRγ (FCER1G), FcγRIIa, FcR β (FcER1b), CD3γ, CD3Δ, CD3ε, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided herein or the equivalent residues from a non-human species, e.g., mouse, rodent, monkey, ape and the like. In a specific CAR of the invention, the primary signaling sequence of CD3-zeta is the sequence as provided herein, or the equivalent residues from a non-human species, e.g., mouse, rodent, monkey, ape and the like.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB/CD137, B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment or derivative thereof.

As used herein, two polypeptide (or nucleic acid) sequences are “substantially different” means that the two sequences are less than 95%, (e.g., 90%, 85%, 80%, 75%, 70%, 65%, 60%, 55%, or 50%) identical to each other.

The term “functional variant” as used herein refers to a modified polypeptide or protein or transgene having substantial or significant sequence identity or similarity to a wild type, such functional variant retaining the biological activity of the wild type polypeptide or protein or transgene of which it is a variant. In some embodiments, functional variants of therapeutic polypeptide or protein or transgenes are used.

A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., one of those described herein) Accordingly, within scope of this invention are hinge regions having one or more point mutations, insertions, deletions, truncations, a fusion protein, or a combination thereof.

Amino acid substitutions can be made, in some cases, by selecting substitutions that do not differ significantly in their effect on maintaining (α) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in the Table below. Amino acid substitutions may be introduced into a parent protein and the products screened for retention of the biological activity of the parent protein.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments f(ab′)2, and fab. F(ab′)2, and fab fragments that lack the Fe fragment of intact antibody, clear more rapidly from the circulation and may have less non-specific tissue binding of an intact antibody (Wahl et al., J. Nucl. Med. 24:316-325 (1983). The antibodies of the invention comprise whole native antibodies, bispecific antibodies; chimeric antibodies; fab, fab′, single-chain v region fragments, fusion polypeptides, and unconventional antibodies.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the n-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single-chain Fv polypeptide antibodies can be expressed from a nucleic acid including VH- and VL-encoding sequences as described by Huston, et al. (Proc. Nat. Acad. Sci., 85:5879-5883, 1988). See, also, U.S. Pat. Nos. 5,091,513, 5,132,405 and 4,956,778; and US patent publication nos. 20050196754 and 20050196754. Antagonistic scFvs having inhibitory activity have been described (see, e.g., Zhao et al., Hybridoma (Larchmont) 2008 27 (6): 455-51; Peter et al., J cachexia sarcopenia muscle 2012 Aug. 12; Shieh et al., J Immunol 2009 183 (4): 2277-85; Giomarelli et al., Thromb Haemost 2007 97 (6): 955-63; Fife et al., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chern 2003 25278 (38): 36740-7; Xie et al., Nat Biotech 1997 15 (8): 768-71; Ledbetter et al., Crit Rev Immunol 1997 17 (5-6): 427-55; Ho et al., Biochim Biophys Acta 2003 1638 (3): 257-66).

“Treating” or “treatment” as used herein refers to administration of a compound or agent to a subject who has a disorder with the purpose to cure, alleviate, relieve, remedy, delay the onset of, prevent, or ameliorate the disorder, the symptom of a disorder, the disease state secondary to the disorder, or the predisposition toward the disorder.

An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “approximately” or “about,” as applied to one or more values, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment. It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXAMPLES

### Example 1. Construction of Anti-NY-ESO-1 TCR-, Anti-NY-ESO-1 TCR-CAR-, Anti-Mesothelin CAR-, and GFP-Based MFG-Retroviral Vectors

### Construction of Retroviral Constructs

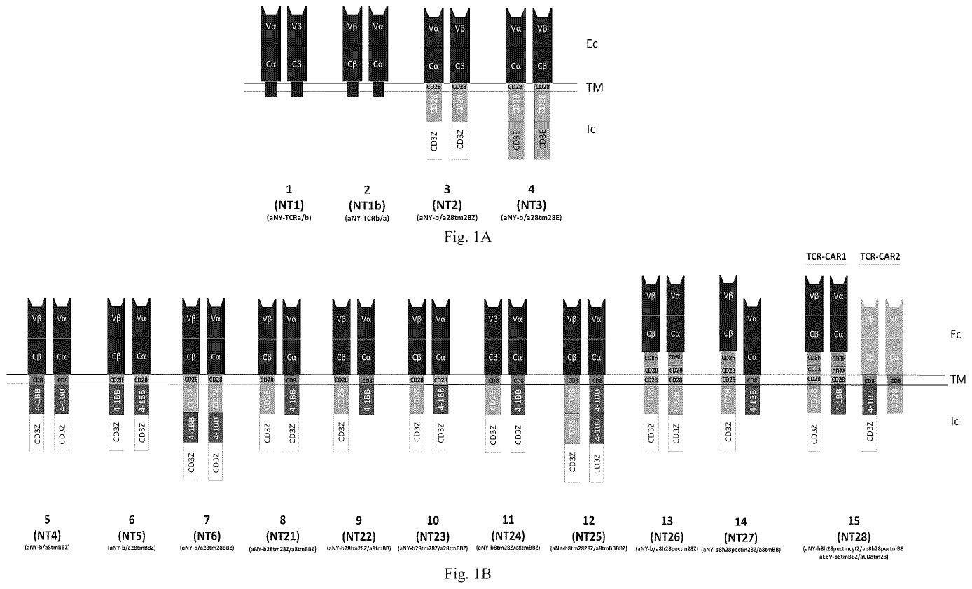

For constitutive expression of genes of interest in T cells, both an MFG retroviral backbone originally developed by Dr. Mulligan (FIG. 1C), and an SFG retroviral backbone (U.S. Pat. No. 6,140,111) were used to construct recombinant retroviral vectors encoding the genes of interest, such a TCR of native form (NT1, NT1b) or TCR-CAR (NT2, NT3, NT4, NT5, NT6, NT21, NT22, NT23, NT24, NT25, NT26, NT27, and T28) specific to human NY-ESO-1 derived peptide/A2 complex (FIGS. 1A and 1B and Table 1).

TABLE 1
List of genes encoding anti-NY-ESO-1 TCR, anti-NY-ESO-1 TCR-CARs, anti-mesothelin CARs, and GFP.

| | Vector | | | SEQ | REF (# |
|---|---|---|---|---|---|
| # | Code | Name | Short Name | ID NO | of FIGS.) |
| 1 | NTI | aNYnativeTCRa/b | aNY-TCRa/b | 9, 12 | 1, 2, 3 |
| 2 | NT1b | aNYnativeTCRb/a | aNY-TCRb/a | 64, 65 | 1, 2, 3b |
| 3 | NT2 | aNY-bCD28tmcytZcyt/ | aNY-b/a28tm28Z | 10, 13 | 1, 2, 4 |
| | | aCD28tmcytZcyt | | | |
| 4 | NT3 | aNY-bCD28tmcytEcyt/ | aNY-b/a28tm28E | 11, 14 | 1, 2, 5 |
| | | aCD28tmcytEcyt | | | |
| 5 | NT4 | aNY-bCD8tmBBcytZcyt/ | aNY-b/a8tmBBZ | 32, 54 | 1b, 16 |
| | | aCD8tmBBcytZcyt | | | |
| 6 | NT5 | aNY-bCD28tmBBcytZcyt/ | aNY-b/a28tmBBZ | 33, 55 | 1b, 17 |
| | | aCD28tmBBcytZcyt | | | |
| 7 | NT6 | aNY- | aNY-b/a28tm28BBZ | 34, 56 | 1b, 18 |
| | | bCD28tmCytBBcytZcyt/ | | | |
| | | aCD28tmcytBBcytZcyt | | | |
| 8 | NT21 | aNY-bCD28tmCytZcyt/ | aNY-b28tm28Z/a8tmBBZ | 35, 57 | 1b, 19 |
| | | aCD8tmBBcytZcyt | | | |
| 9 | NT22 | aNY- | aNY-b28tm28Z/a8tmBB | 36, 58 | 1b, 20 |
| | | bCD28tmCytBBcytZcyt/ | | | |
| | | aCD28tmcytBBcyt | | | |
| 10 | NT23 | aNY-bCD28tmcytZcyt/ | aNY-b28tm28Z/a28tmBBZ | 37, 59 | 1b, 21 |
| | | aCD28tmBBcytZcyt | | | |
| 11 | NT24 | aNY- | aNY-b8tm28Z/a8tmBBZ | 38, 60 | 1b, 22 |
| | | bCD8tmCD28cytZcyt/ | | | |
| | | aCD8tmBBcytZcyt | | | |
| 12 | NT25 | aNY-bCD8tmcytcytZcyt/ | aNY- | 39, 61 | 1b, 23 |
| | | aCD8tmBBcytcytZcyt | b8tm2828Z/a8tmBBBBZ | | |
| 13 | NT26 | aNY- | aNY-b/a8h28pectm28Z | 40, 62 | 1b, 24 |
| | | bCD8hCD28pectmcytZcyt/ | | | |
| | | aCD 8hCD28pectmcytZcyt | | | |
| 14 | NT27 | aNY- | aNY- | 41, 63 | 1b, 25 |
| | | bCD8hCD28pectmcytZcyt/ | b8h28pectm28Z/a8tmBB | | |
| | | aCD8tmBBcyt | | | |
| 15 | NT28 | aNY- | aNY- | | 1b |
| | | bCD8hCD28pectmcytZcyt/ | b8h28pectmcytZ/ab8h28pe | | |
| | | aCD8hCD28pectmBBcyt | ctmBBaEBV- | | |
| | | aEBV- | b8tmBBZ/aCD8tm28 | | |
| | | bCD8tmBBcytZcyt/aCD8t | | | |
| | | mCD28cyt | | | |

The GFP was used as marker to chase GFP-based vector-transduced T cells by flow cytometry (FACS) and/or by fluorescent microscope. The anti-human mesothelin sFv (GenBank ID: AF035617.1) with high affinity for human mesothelin (Chowdhury P S et al. Proc Natl Acad Sci USA. 1998; 95:669-74) was chosen for the construction of anti-mesothelin-based CARs. Such usage of anti-mesothelin sFv was described previously in successfully creating functional anti-mesothelin CARs of 2^{nd} and 3^{rd} gen (Carpenito C. et al. Proc Natl Acad Sci USA. 2009; 106:3360-5).

Recombination between nucleic acids is a well-established phenomenon in molecular biology. Genetic recombination that requires strong sequence homology between participating nucleic acid sequences to occur is generally referred to as homologous recombination. While most genetic knockout strategies employ homologous recombination to achieve a targeted knockout, in certain systems the occurrence of genetic recombination can impact genetic manipulations detrimentally. In particular, homologous recombination events can adversely impact construction and production of vectors, particularly viral vectors (e.g., adenovirus, retrovirus, adeno-associated virus, herpes virus, etc.), where it is often desirable to maintain highly homologous sequences (e.g., identical polypeptide sequences) within a single, stable viral vector free of homologous recombination during, e.g., passage and/or propagation of viral vector through one or more host cells and/or organisms.

An approach previously described (U.S. Pat. No. 9,206,440) was employed for the vector designs for overcoming such problems associated with potential homologous recombination between nucleic acids is to enable delivery of two or more nucleic acid sequences encoding for highly homologous (e.g., identical) polypeptide fragments in a single protein molecule on a single viral vector. In such an approach, the silent mutation method at the nucleotide sequence level is employed to produce viral vector sequences comprising of nucleic acid sequences encoding two or more highly homologous (e.g., identical) polypeptides or polypeptide domains thereof, yet that possess reduced risk of homologous recombination between such nucleic acid sequences, even during, e.g., extended passage in host cells and/or multiple infection, chromosomal integration, and/or excision events.

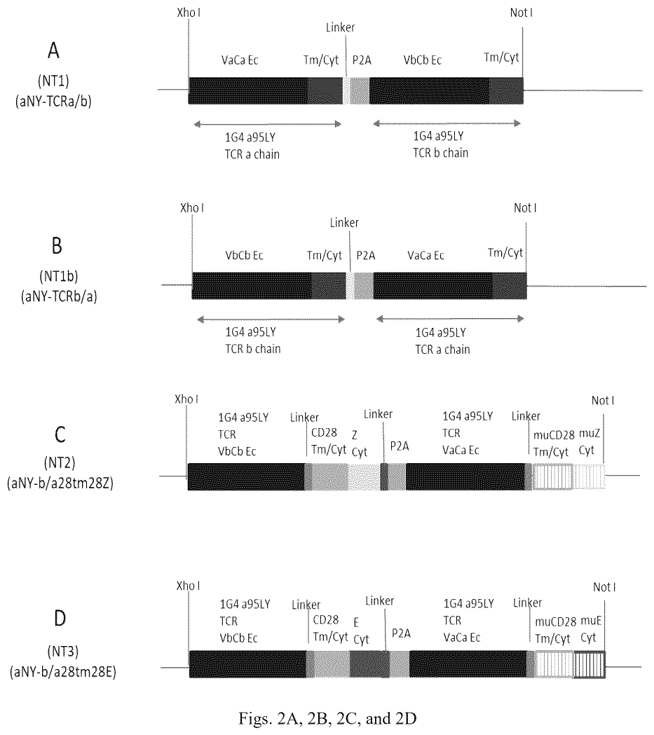

A mutated TCR that recognizes the peptide SLLMWITQC (SEQ ID NO: 25), corresponding to residues 157 to 165 of NY-ESO-1 (NY-ESO-1:157-165) with high affinity, in the context of the HLA-A*0201 class I restriction element, was previously described (Robbins P F et al., J Clin Oncol. 2011; 29:917-24; and Robbins P F et al., J Immunol. 2008; 180:6116-6131). This TCR, termed 1G4-α95LY, contains two amino acid substitutions in the third complementarity determining region of the native 1G4 TCR α chain that conferred to CD8+ and CD4+ T cells, an enhanced ability to recognize HLA-A*0201-positive target cells expressing the NY-ESO-1 antigen. The entire alpha chain and beta chain (SEQ ID NO: 1 and SEQ ID NO: 2), or their extracellular domains of the TCR IG4-a95LY (SEQ ID NO: 4 and SEQ ID NO: 5) were initially made use of for the generation of two anti-NY-ESO-1 TCRs of native form IG4-a95LY TCR (NT1, NT1b) (SEQ ID NO: 9, 64) or two TCR-CARs (IG4-a95LY TCR-CAR) (NT2; IG4-a95LY TCR-based CAR-CD28Z (aNY-b/a28tm28Z), (SEQ ID NO: 10), and NT3; IG4-a95LY TCR-based CAR-CD28E (aNY-b/a28tm28E) (SEQ ID NO: 11) (FIGS. 1 and 2), that recognize the peptide SLLMWITQC (SEQ ID NO: 25), corresponding to residues 157 to 165 of NY-ESO-1 (NY-ESO-1:157-165) with high affinity, in the context of the HLA-A*0201 class I restriction element.

The above-mentioned MFG retroviral backbone was employed to construct recombinant viral vector encoding one of two anti-NY-ESO-1 TCR of native forms (Vectors 1 and 2; FIGS. 1A, 1B), one of twelve anti-NY-ESO-1 TCR-based CARs (Vectors 3-15; FIGS. 1A, 1B), or anti-human mesothelin-CAR, or GFP.

### (1) 1G4-a95LY TCR-Vectors of Anti-NY-ESO-1/A2 of Native Form (FIGS. 1A, 1B, 2A, 2B):

Vector 1 (NT1, NT1a, aNY-TCRa/b) (SEQ ID NOs: 9 and 12)

A MFG-based retroviral vector encoding a TCR that recognizes the peptide SLLMWITQC (SEQ ID NO: 25) in the context of the HLA-A*0201 class I restriction element, was generated in the retroviral vector backbone as previously described (Robbins P F et al., J Clin Oncol. 2011; 29:917-24 and Robbins et al., J Immunol. 2008; 180:6116-6131), but in the MFG-based vector. This TCR, termed 1G4-a95:LY, contained two amino acid substitutions in the third complementarity determining region of the native 1G4 TCR α chain that conferred to CD8 and CD4 T cells, an enhanced ability to recognize HLA-A*0201-positive target cells expressing the NY-ESO-1 antigen (Robbins P F et al, J Immunol. 2008; 180:6116-6131).

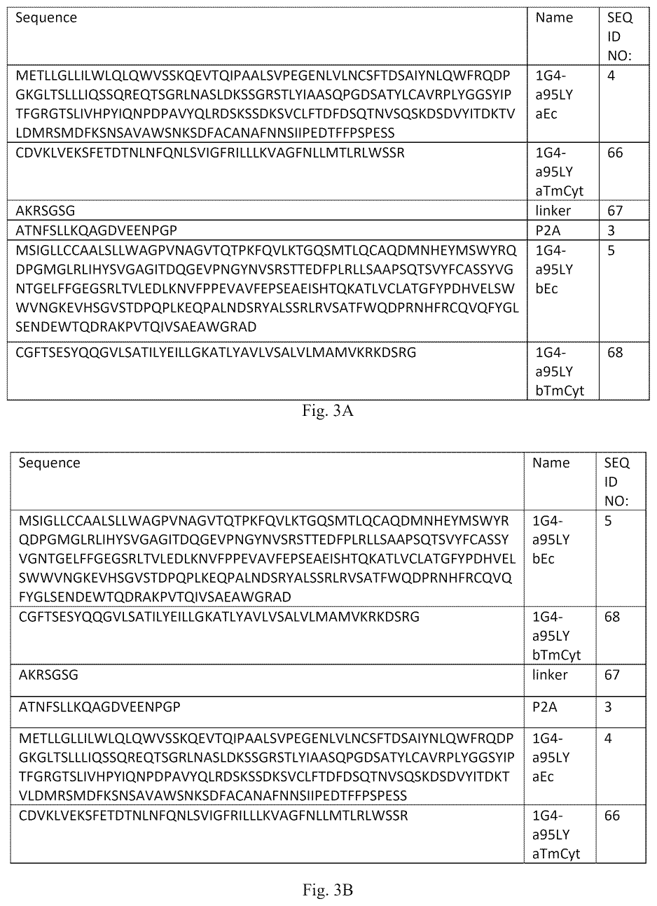

The 1G4 α- and β-chains were expressed in retroviral constructs that contained the “self-cleaving” P2A sequence (Szymczak et al., Nat. Biotechnol. 2004; 22:589-594) between the two gene products. The peptide sequence of the NT1 (aNY-TCRa/b) (SEQ ID NO: 9) (FIG. 3) was designed to be encoded by a nucleotide sequence (SEQ ID NO: 12). To facilitate the molecular subcloning of the gene of interest in the cloning site of the MFG-based vector, an XhoI site (CTCGAG, SEQ ID NO: 71), and a short sequence (CAGCCAGCGGCCGC, SEQ ID NO: 72) comprising of a NotI site (GCGGCCGC, SEQ ID NO: 73) were inserted immediately upstream of the “ATG” start codon and downstream of the stop codon “TAA” in the coding region, respectively (SEQ ID NO: 12).

Vector 2 (NT1b; aNY-TCRb/a)

Vector 2 (NT2; aNY-TCRb/a) (SEQ ID NOs: 64 and 65) was designed exactly the same as Vector 1 (NT1; aNY-TCRa/b) except that the DNA fragments encoding the native form of the TCR's alpha chain is located immediate downstream of the 3′ end of the P2A encoding sequence while the beta chain located upstream of the P2A encoding sequence (FIGS. 1, 2, and 3).

(2) 1G4-a95LY TCR-CAR Integrated with CD28 and CD3Z Signaling-Vector (NT2, NT3) (FIG. 1C and FIG. 1D)
Vector 3 (NT2; aNY-b/a28tm28Z) (2^{nd} Gen)

A recombinant retrovirus encoding two or more molecules that are identical or highly homologous at nucleic acid sequences may lead to homologous recombination, and as a result, genomic rearrangements, such as deletions and duplications of homologous genes may occur. For example, nucleic acid sequences encoding the Tm and Cyt domains of TCR-based CARs such as CD28TmCyt, CD3ZCyt and CD3ECyt segments may cause unwanted mutations and/or deletion of genes encoding the TCR-CARs during plasmid DNA and retroviral vector preparation processes. To address this problem, mutation procedures can be applied to suppress homology driven recombination between the repeated segments in the same vector.

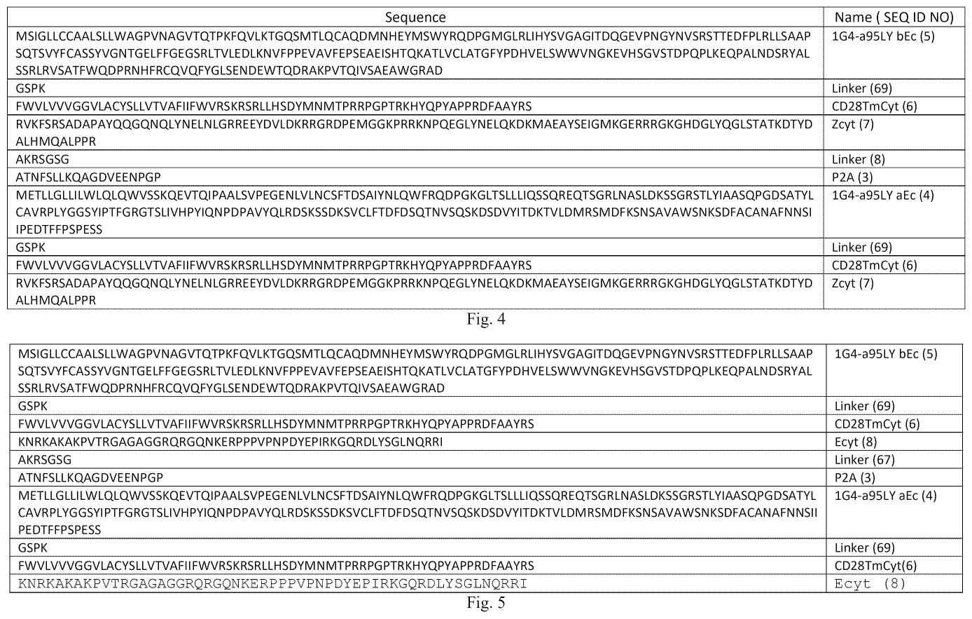

The 28Z or 28E peptide sequence was preceded by the amino acids GSPK (SEQ ID NO: 69) as a linker (FIGS. 4 and 5), and linked to TCRa (TCR-Ca amino acids ending at SPESS) and TCRb (TCR-Cb amino acids ending at WGRAD) as previously described (Govers et al., Journal of Immunology, 2014, 193:5315-5326).

The NT2 (Vector 3; aNY-b/a28tm28Z (1G4 a95LY TCR-based CAR) was designed to comprise 28Z signaling (FIG. 1C and FIG. 2C) (SEQ ID NO: 10).

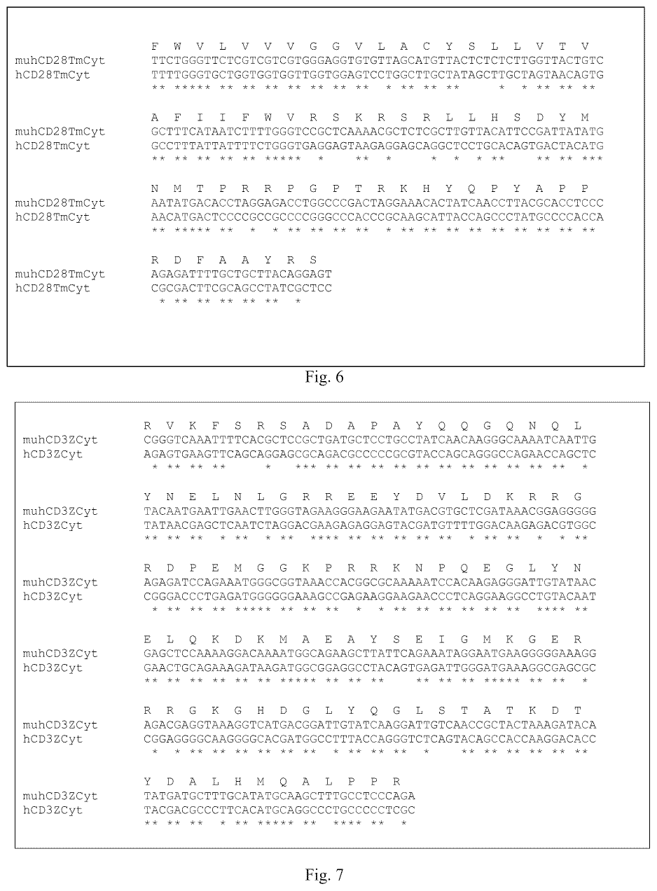

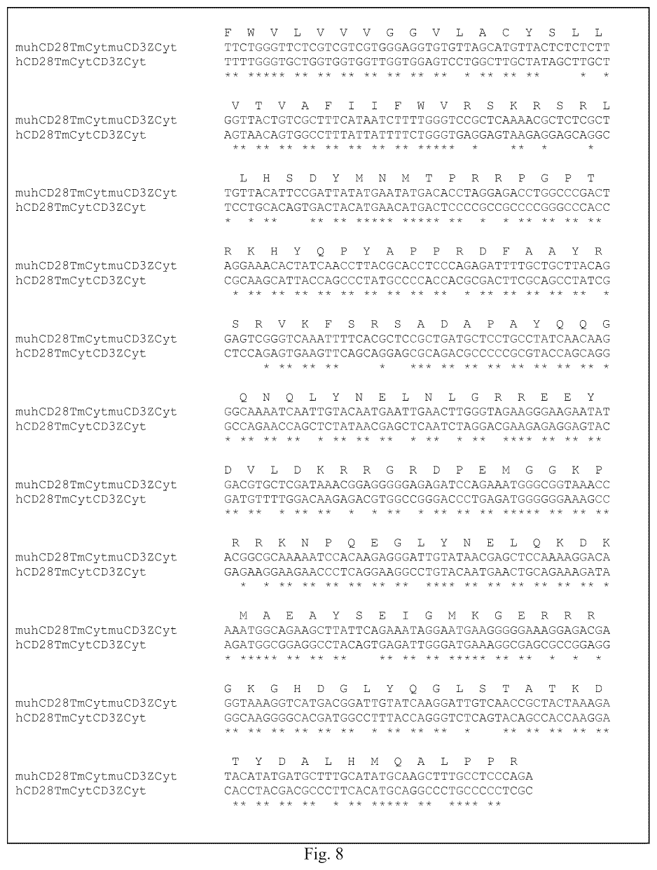

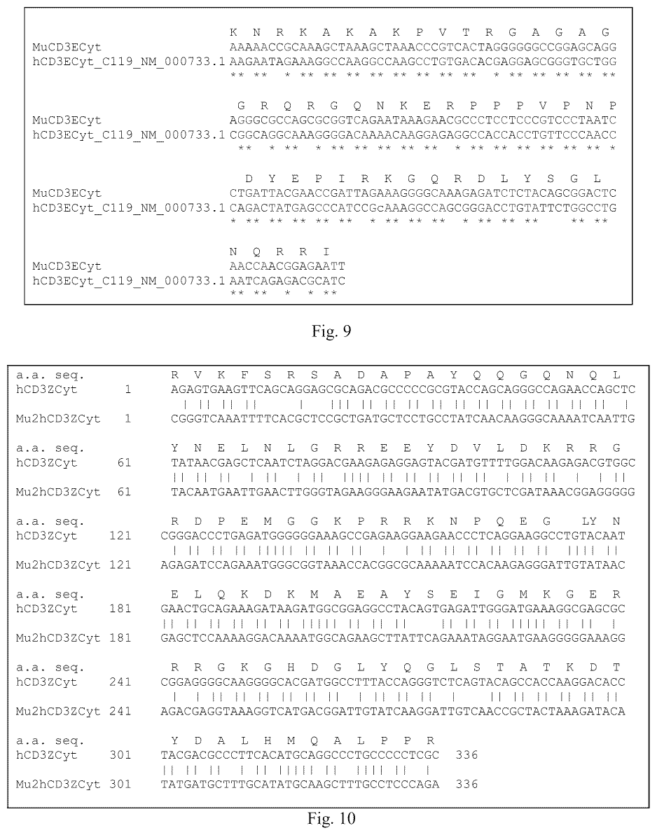

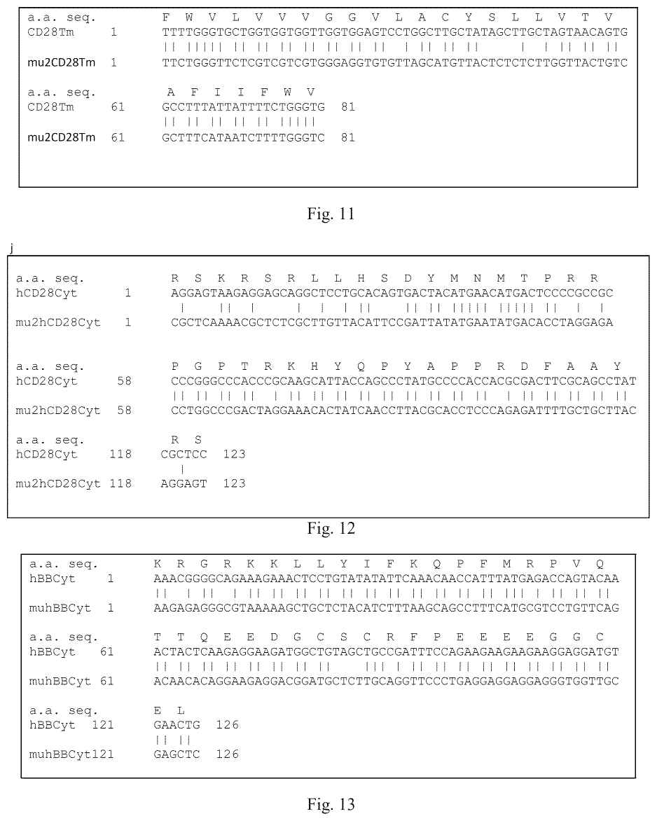

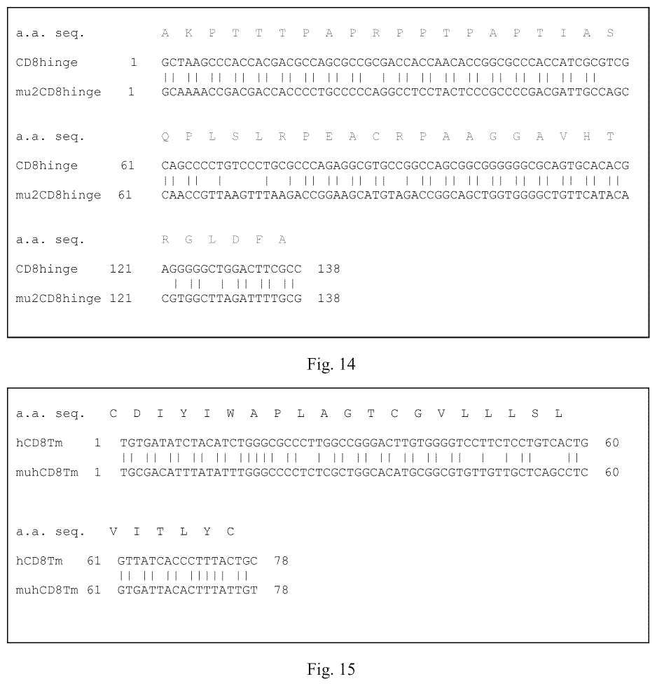

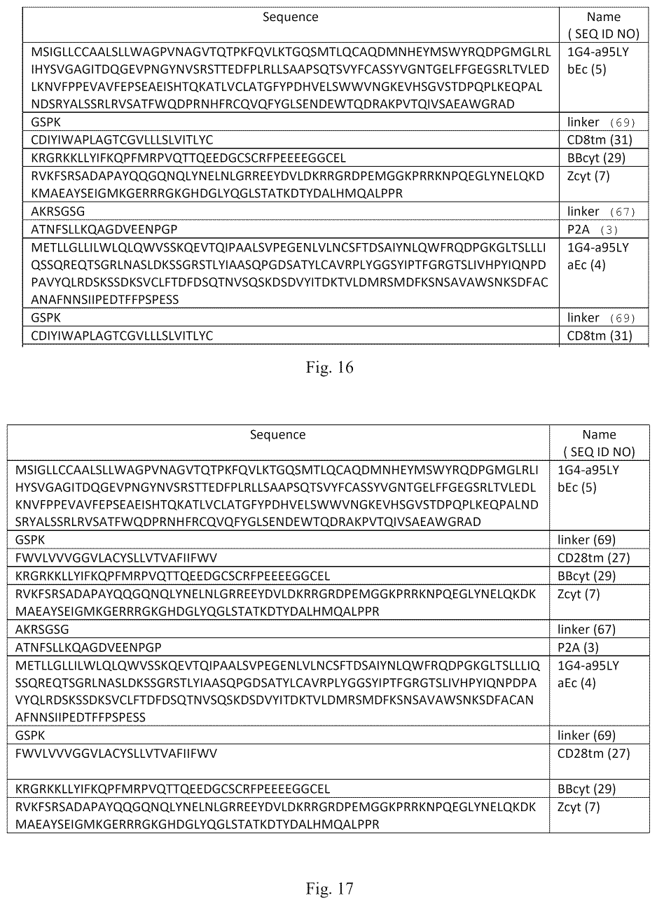

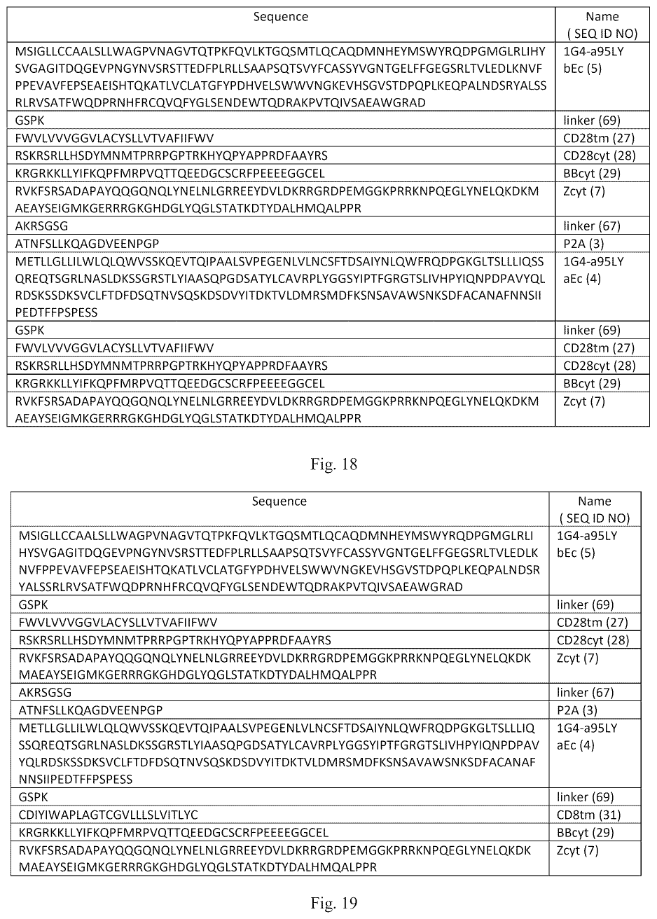

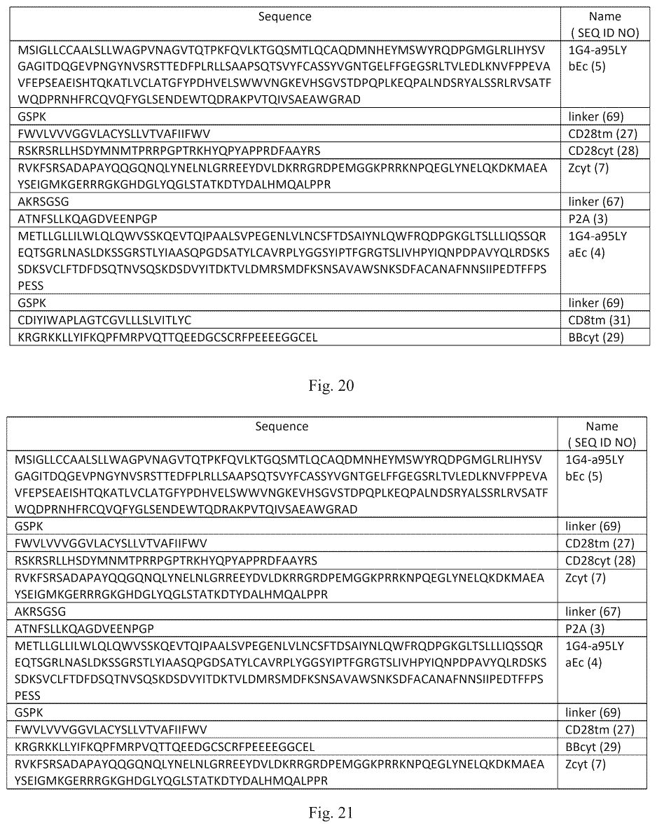

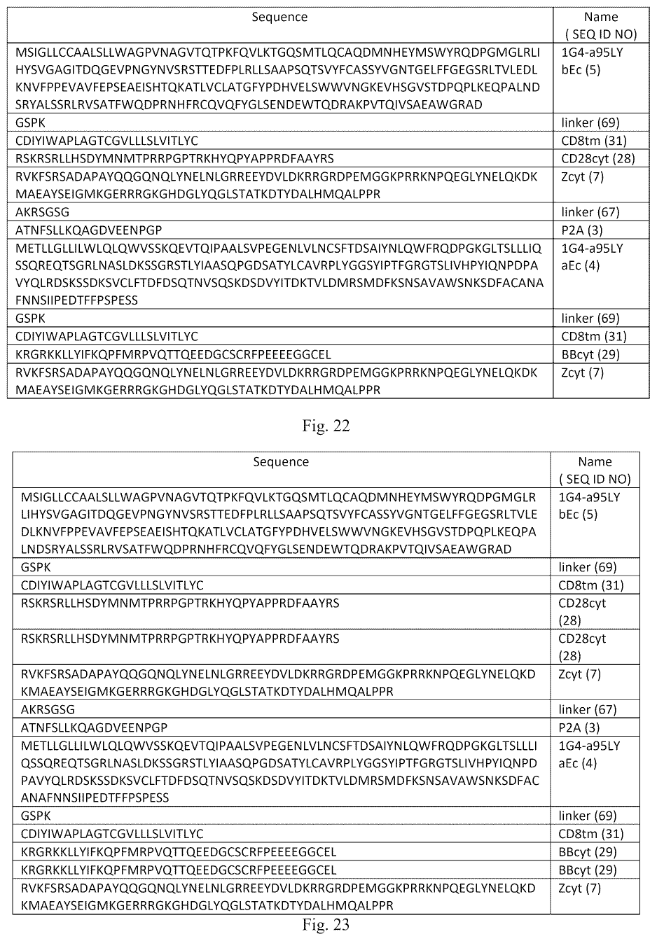

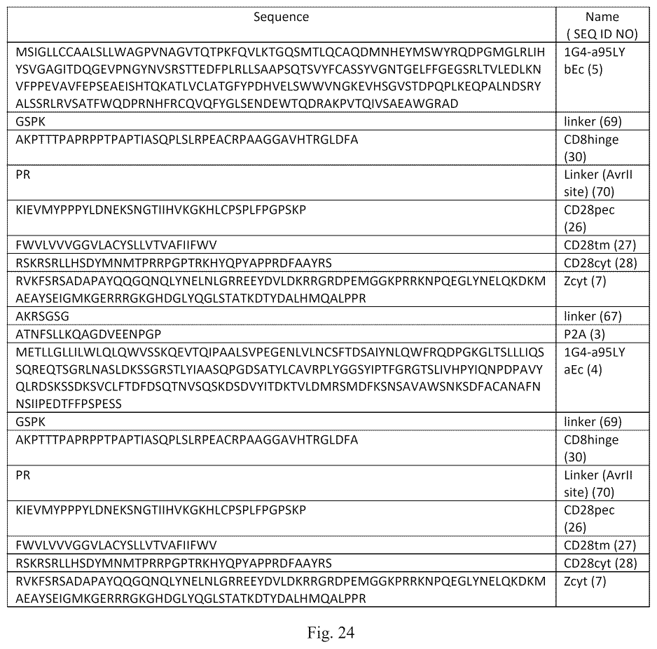

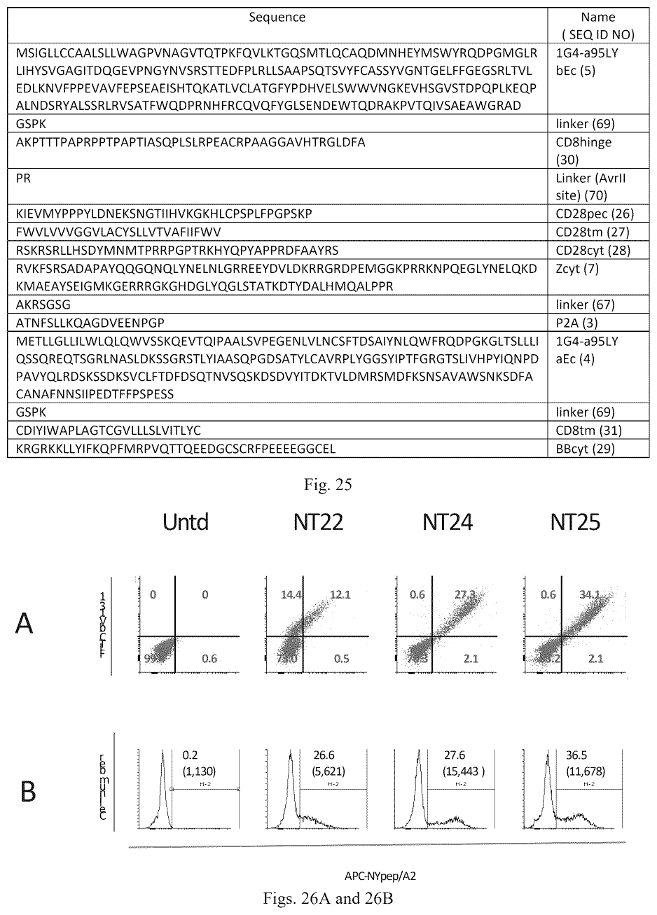

To construct NT2 (FIG. 2C; SEQ ID NO: 10, 13), the extra cellular domain of the beta chain (SEQ ID NO: 5) and the extra cellular domain of the alpha chain (SEQ ID NO: 4) were molecularly linked, via a linker (amino acids GSPK (SEQ ID NO: 69) to the Tm and Cyt of human CD28 (28TmCyt) (SEQ ID NO: 6) and the Cyt of human CD3Z (ZCyt) (SEQ ID NO: 7), in which the CD28Z (SEQ ID NO: 15) immediately linked to the extra cellular domain of 1G4 a95LY TCR alpha chain were silently mutated without alternation of its original amino acid sequence (mu28muZ) (FIGS. 6-8) (SEQ ID NO: 21), in order to reduce the homology at the level of nucleotide sequences encoding the 28Z between those linked immediately to the C-terminus of Ec of the beta chain and alpha chain. Similar to the 1G4 a95LY TCR for native form (NT1, and NT1b), a “self-cleaving” P2A sequence (SEQ ID NO: 3) was also inserted in between the betaCD28Z and alphaCD28muZ (FIG. 4).

Vector 13 (NT26; aNY-b/a8h28pectm28Z) (2^{nd} Gen)

Vector 13 (NT26, SEQ ID NOs: 40 and 62) was designed and created in the same manner as Vector 3 (NT2) except that two DNA sequences encoding a same amino acid sequence of a human CD8α hinge (CD8h) (SEQ ID NO: 30) and partial human CD28 extracellular domain (CD28pec) (SEQ ID NO: 26) were inserted between the amino acids GSPK (SEQ ID NO: 69) linked to the C-terminal of Cb and N-terminal of CD28Tm (SEQ ID NO: 42, 50), and between the amino acids GSPK (SEQ ID NO: 69) linked to the C-terminal of Ca and N-terminal of CD28Tm (SEQ ID NO: 43,51).

(3) 1G4-a95LY TCR-CAR Integrated with CD28 and CD3E Signaling-Vector (NT3; aNY-b/a28tm28E) (FIGS. 1 and 2)
Vector 4 (NT3; aNY-b/a28tm28E) (2^{nd} Gen)

The design of the 28E cassette at amino acid sequence level was identical to that described previously, covering the Tm and Cyt (Ic) domains of human CD28 (GI: 338444, aa 153-220, numbering starting from first methionine), followed by the IC domain of human CD3ε (GI: 4502670, aa 153-207) (Tan Van et al., J Immunol 2014; 193:5315-5326). The 288 cassette was preceded by the amino acids GSPK (SEQ ID NO: 69) and linked to the downstream of the Ec of the C alpha region (TCR-Ca amino acids ending at SPESS) and TCRb (TCR-Cb amino acids ending at WGRAD). A nucleotide sequence encoding the NT3 (SEQ ID NO: 14) comprising of a nucleotide sequence of Xho I immediately upstream of the coding region of the aNY-TCR28E site and a short fragment of 3 prime end franking and a Not I site were designed (FIG. 2D, FIG. 5, and FIG. 11) (SEQ ID NO: 14) (SEQ ID NO: 11) and commercially synthesized (BIO BASIC CANADA INC, Canada). G4 a95LY TCR-based CAR comprising 28E signaling (NT3; 1G4 a95LY TCR-28E) (aNY-b/a28tm28E) (FIG. 1D and FIG. 2D)

Similar to the aNY-TCR28Z (NT2; Vector 3), to construct IG4 a95LY TCR-based CAR comprising 28E signaling (1G4 a95LY TCR-28E; aNY-TCR28E; Vector 3), the Ec domains of the beta and alpha chains were molecularly linked, via a linker (amino acids GSPK (SEQ ID NO: 69), to the Tm domain and Cyt domain of human CD28 and Cyt of human CD3E, in which the CD28E immediately linked to the extra cellular domain of 1G4 a95LY TCR alpha chain were silently mutated without alteration of its original amino acid sequence (mu28muE) (FIG. 6), in order to reduce the homology at the level of nucleotide sequences encoding the 28E between those linked immediately to the C-termini of Ec domains of the beta chain and alpha chain. Similar to the 1G4 a95LY TCR, a “self-cleaving” P2A sequence (SEQ ID NO: 3) was also inserted in between the betaCD28E and alphamuCD28muE (FIG. 5).

(4) 1G4-a95LY TCR-CAR Integrated with 4-1BB and CD3Z Signaling-Vectors (Vectors 5 (NT4; aNY-b/a8tmBBZ) and 6 (NT5; aNY-b/a28tmBBZ)
Vector 5 (NT4; aNY-b/a8tmBBZ) (2^{nd} Gen)

Vector 5 (SEQ ID NOs: 32 and 54) was designed and created in the same manner as Vector 3 (NT2) except that the DNA sequences encoding the Tm and Cyt domains of CD28 were replaced by DNA sequences encoding the Tm domain of human CD8 (SEQ ID NO: 31) (SEQ ID NOs: 52 and 53) and the Cyt domain of human 4-1BB (SEQ ID NO: 29) (SEQ ID NO: 48,49).

Vector 6 (NT5; aNY-b/a28tmBBZ) (2^{nd} Gen)

The vector 6 (NT5; aNY-b/a28tmBBZ) (SEQ ID NO: 33,55) was designed and created exactly the same as Vector 5 (NT4) except that the DNA sequences encoding the Tm domain of CD8 were replaced by DNA sequences encoding the Tm domain of human CD28 (SEQ ID NO: 29) (SEQ ID NOs: 48 and 49).

(5) 1G4-a95LY TCR-CAR Integrated with a Combination of CD28 and 4-1BB and CD3Z Signaling-Vectors (Vectors 7 (NT6), 8 (NT21), 9 (NT22), 10 (NT23), 11 (NT24), 12 (NT25), 14 (NT27).
Vector 7 (NT6; aNY-b/a28tm28BBZ) (3^{rd} Gen)

Vector 7 (SEQ ID NO: 34, 56) was designed and created in the same manner as Vector 3 (NT2) except that the DNA sequences encoding the Cyt domain of human 4-1BB (SEQ ID NO: 29) (SEQ ID NOs: 48 and 49) were inserted between the Cyt domains of the C-terminal of CD28 and N-terminal of CD3Z, respectively.

Vector 11 (NT24; aNY-b8tm28Z/a8tmBBZ) (3^{rd} Gen)

Vector 11 (SEQ ID NO: 38, 60) was designed and created in the same manner as Vector 5 (NT4; aNY-b/a8tmBBZ) except that DNA sequence encoding the Cyt domain of 4-1BB located between the C-terminus of CD8Tm and N-terminus of CD3ZCyt in the first polypeptide comprising the Ec domain of TCR beta chain (i.e., upstream of P2A peptide) was replaced by a DNA sequence encoding the Cyt domain of human CD28.

Vector 12 (NT25; aNY-b8tm2828Z/a8tmBBBBZ) (3^{rd} Gen)

Vector 12 (SEQ ID NOs: 39 and 61) was designed and created in the same manner as Vector 11 (NT24) except that an additional DNA sequence encoding the Cyt domain of CD28 (2^{nd} unit of CD28Cyt) was inserted in between the C-terminal of CD8Tm and N-terminal of CD28Cyt in the 1^{st} polypeptide comprising the Ec the anti-NY-ESO-1TCR beta chain, and an additional DNA sequence encoding the Cyt domain of 4-1BB (2^{nd} unit of 4-1BBcyt) was inserted in between the C-terminal of CD8Tm and N-terminal of 41B-B in the 2^{nd} polypeptide comprising the Ec of the anti-NY-ESO-1TCR alpha chain.

Similar molecular designs and approaches as described above were employed to design and create the other vectors including Vector 8 (SEQ ID NOs: 35, 57; NT21; aNY-b28tm28Z/a8tmBBZ), Vector 9 (SEQ ID NOs: 36, 58; NT22; aNY-b28tm28Z/a8tmBB) Vector 10 (SEQ ID NOs: 37, 59; NT23; aNY-b28tm28Z/a28tmBBZ), Vector 14 (SEQ ID NOs: 41, 63; NT24; aNY-b8h28pectm28Z/a8tmBB) (Table 1), accordingly.

The DNA fragments encoding the fourteen anti-NY-ESO-1 TCRs or TCR-CARS (Vectors 1-14; Table 1), were commercially synthesized (BIO BASIC CANADA INC, Canada), and subsequently sub-cloned into XhoI/NotI sites of the MCS region of the MFG-based retroviral vector (FIG. 1C) in the manner as previously described (Yang W et al. Int Immunol. 2007; 19:1083-93). All vector inserts encoding TCR or TCR-CARs were verified by DNA sequencing.

### Example 2. Generation of Retrovirus Encoding Relevant Gene Product of Interest

Phoenix ampho cells (a 293 cell derivative line with high calcium phosphate transfection efficiency) were transfected individually with the fourteen vectors (Table 1; FIGS. 1, 2). Viral supernatants (containing virions) of the transfected Phoenix cells were collected, immediately used to infect PG13 cells or other mouse cells, or stored in −80° C. for future use, followed by standard protocols known in the art such as those described in Yang W et al., Int Immunol. 2007; 19:1083-93 and Beaudoin E L et al., J Virol Methods 2008; 148:253-9.

PG13 cells, a viral producing cell (VPC) line, were infected with the retroviral supernatant obtained from Phoenix cells transfected separately with vectors of interests. FACS-based cell sorting was conducted to enrich corresponding vector-infected PG13 cells. Infected PG13 cells were enriched by cell sorting for human TCR Vβ13.1 chain positive cells after staining with FITC anti-human TCR Vβ13.1 Antibody (EBIOSCIENCE, AFFYMETRIX, and THERMO FISHER SCIENTIFIC), or NYpep/A2 tetramer (Tetramer/APC-HLA-A*02:01 NY-ESO-1 (SLLMWITQC; SEQ ID NO: 25) (Fred Hutchinson Cancer Research Center, Seattle, WA 98109, USA) positive cells as previously described. Production of high titer retrovirus supernatant with enriched VPCs followed standard protocols known in the art.

In one instance, prior to FACS-based cell sorting of enrichment of TCR+ or TCR-CAR+ PG13 cells after viral infection, the infected PG13 cells were subjected to FACS analysis. PG13 cells were infected with a retroviral supernatant obtained from Phoenix cells transfected separately with vectors encoding three anti-NY-ESO-1 TCR-CARs (NT22, NT24, and NT25). The infected PG13 cell lines (without previous enrichment for CAR+ cells by FACS-based cells sorting) were analyzed for surface expression of TCRVb13.1 and binding of NYpep/A2 tetramer by FACS. Live cells were gated for analysis. Shown in FIG. 26 are Intensity plots showing positive staining of single or double positive cells to FITC-anti-human TCRVb13.1 and APC-NYpep/A2 tetramer, individually.

### Example 3. Generation of Activated Human T Cells Transduced Individually with Retrovirus

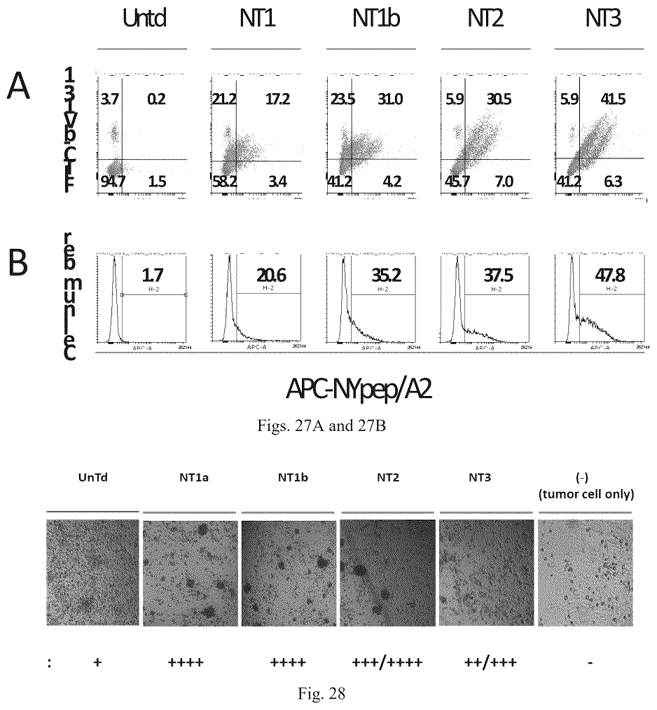

Anti-CD3 antibody-activated human T cells (ATC) derived PBMC were individually transduced with each of seven vectors: aNY-TCRs (NT1, NT1b) and aNY-TCR-CARs (NT2, NT3, NT4, and NT5, NT6) (Table 1) in the manner described in Yang W et al., Int Immunol. 2007; 19:1083-9. Seven to ten days post transduction, flow cytometry analysis was conducted by staining with a combination of FITC-anti-human TCR Vβ13.1 and Tetramer/APC-HLA-A*02:01 NY-ESO-1 (SLLMWITQC, SEQ ID NO: 5) (Fred Hutchinson Cancer Research Center, Seattle, WA). The results are shown in FIG. 27.

As shown in the figure, the two 2^{nd} gen anti-NYESO-1/A2 TCR-CARs (NT2 and NT3) expressed at significantly higher levels on the transduced ATC (including CD4+ and CD8+ cells) cells both for human TCRVb13.1 and NYpep/A2 tetramer-binding TCRs compared to the native forms (NT1 and NT1b). Approximately 3.9% of the un-transduced cells (Untd) were also positive for TCRVb13.1 staining, but these cells do not significantly stained with APC-NYpep/A2 tetramer.

It was found that between 19.4-51.2% of activated T cells bound to the Tetramer/APC-HLA-A*02:01 NY-ESO-1. The expression of TCR Vβ13.1 on their cell surface was measured based on FITC anti-human TCR Vβ13.1 antibody (EBIOSCIENCE/AFFYMETRIX, CA) staining and similar percentages of activated T cells expressed correct pairs of functional 1G4 a95LY TCR Ec domain on the cell surface based on positive tetramer staining with Tetramer/PE-HLA-A*02:01 NY-ESO-1 followed by FACS analysis. Further analysis indicated that approximately half of CAR+CD3+ cells of infected activated human T cells were CD4+ (52%) and CD8+ (48%).

### Example 4. Antigen Specific-Mediated Secretion of Cytokine IL-2 of Human ATC Transduced with Anti-NY-ESO-1 TCR or One of the Two TCR-Based CARs

In this example, the activity of inducing cytokine secretion by activated human T cells was examined by detecting IL-2 secretion in media where the T cells were co-cultured with HLA-A0201+ target cells in the absence and presence of various amounts of specific antigen peptides.

Briefly, 0.5×10⁵ of HLA-A0201+ target cells (T2 cells, 174×CEM.T2, ATCC CRL-1992™) were incubated in absence or presence of various concentrations of peptide NY-ESO-1 (SLLMWITQC, SEQ ID NO: 25) at 10 nM, 100 nM and 2 uM) in culture medium for 3 hours at 37° C. The cells were washed twice to remove unbound peptides and then co-cultured with 1.5×10⁵ activated T cells that were transduced individually with vectors NT1 (NT1a), NT1b, NT2 and NT3 (FIG. 2) in 200 ul of culture medium (complete RPMI 1640+10% FCS+Pen/Strep) (R-10) per well of a U-bottom 96-well plate for 24 hours.

The cultured supernatants were collected and measured for IL-2 concentrations by ELISA detection kit (EBIOSCIENCES). Resulting IL-2 secretion data show that activated T cells transduced individually with all three anti-NY-ESO-1 vectors NT1 (NT1a), NT1b, NT2 and NT3 (FIG. 2) specifically secreted IL-2, upon engagement with the NY-ESO-1 peptide-pulsed-T2 cells, but not T2 cells un-pulsed with the NY-ESO-1 peptide, or pulsed with negative control peptides.

It was found that the T cells transduced with NT2 secreted the highest amounts of IL-2 among the three while the T cells transduced with NT1 produced the lowest amount at the same concentrations of peptide at which T2 cells were pulsed. These results indicate that both the two TCR-CARs (NT2 and NT3) were superior to the NT1 (FIG. 2A) in transduced TCR- or TCR-CAR mediated T cell activation.

### Example 5. Specific Killing of Tumor Cells by ATC Transduced with Anti-NY ESO-1 TCR or One of the Two TCR-Based CARs

In this example, assays were carried out to examine activities of killing tumor cell by activated human T cells (ATC) transduced with anti-NY-ESO-1 TCR or TCR-based vectors NT1, NT1b, NT2 and NT3 (FIG. 2) using Saos-2 cells (ATCC® HTB-85™). Saos-2 cell line, a human osteosarcoma cell line, is NY-ESO-1+ and HLA-A0201+.

Briefly, 1×10³ Saos-2 cells were pre-plated on to a well of a 96-flat well tissue culture plate for 12 hours. Then, 1×0⁴ un-transduced or transduced human T cells were added to each well. These T-cells had been un-transduced or transduced individually with the three vectors, NT1, NT2 and NT3 in a complete RPMI 1640+10% FCS+Pen/Strep medium (R10) complemented with 90 IU of rec hu IL-2/ml). The cells were cultured for up to 5 days (120 hrs.) (FIG. 28). The survival tumor target cells, which were profoundly larger than and distinct from T cells, attached to the bottom of wells, and were observed and graded daily.

The killing activity of T cells was graded as: “++++”, 75-100% killing; “++++”, 75-100% killing; “+++”, 50-75% killing; “++”, 25-50% killing; “+”, 0-25% killing, by comparing the surviving tumor cell number from the wells with T cells to those from the wells without T cells. Photos of representative views of wells under microscope were taken at day 5 (120 hrs) post the addition of T cells. See FIG. 28.

As shown in the figures, ATCs expressing aNY-TCRa/b (NT1 (NT1a) and NT1b) exhibited similar, highest tumor-killing activities. ATC-expressing NT2 exhibited slightly lower killing activity, and those with NT3 showed relatively lower killing ability. Un-transduced ATC showed no notable killing. These results suggest that human T cells transduced individually with the four vectors can specifically kill the Saos-2 tumor cells.

### Example 6. Antigen Specific-Mediated Secretion of Cytokine IL-2 and INF-γ

In this examples, assays were carried out to examine cytokine secretion by ATC transduced with one of the two anti-NY ESO-1 TCR (NT1 (NT1a) and NT1b) or one of the two 2^{nd} gen TCR-based CARs (NT2 and NT3).

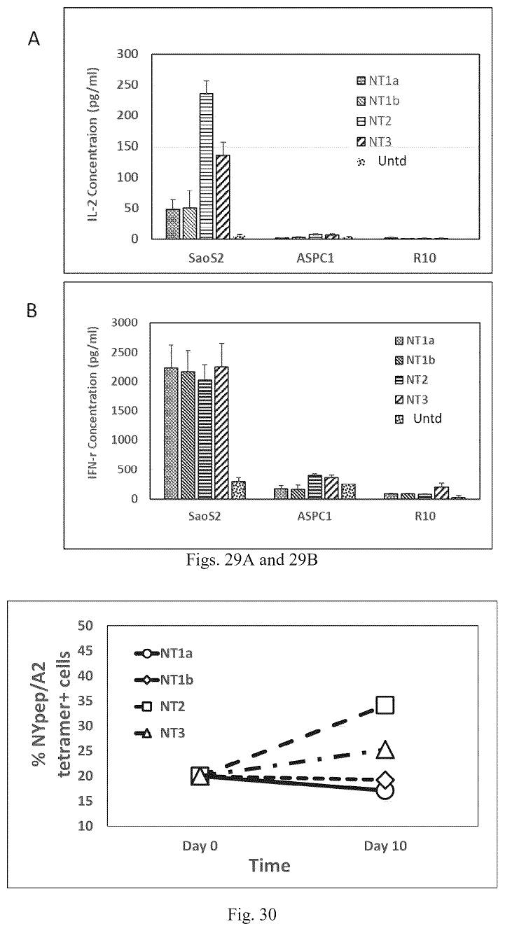

ATC transduced with NT1a, NT1b, NT2, or NT3 were prepared in the manner described above. The percentages of T cells that were NYpep/A2 positive based on APC-NYpep/A2 tetramer staining by FACS (for NT1a, NT1b, NT2, and NT3) were adjusted to 20% with un-transduced T cells (“untd”) as describe above. Then, 0.5×10⁶ target cells (Saos-2 or control AspC1) or media only (RPMI-1640+10% FCS+Pen/Strep) (R10) without any IL-2 added) were co-cultured with 2×10⁶ of the ATCs transduced with NT1a, NT1b, NT2, or NT3 in a 24-flat well tissue culture plate in a total volume of 1.5 ml of R10 (without any IL-2 added) for 24 hours. Concentrations of IL-2 or INF-γ in the cell supernatants were measured by ELISA with kits from BIOLEGENDS (Cat #s. 431804 and 430101). The results are shown in FIG. 29. As shown in FIG. 29A, T cells transduced with NT2 secreted the highest amount of IL-2 (about 230 pg/ml), while those with NT3 secreted a moderate amount of IL-2, and those with either of the two native form of anti-NY-ESO-1/A2 TCR (NT1 and NT1b) secreted the lowest amount.

### Example 7. Changes of Percentages of NYpep/A2+ Cells of ATC Transduced with NT1, NT1b, NT2 and NT3

ATC transduced with one of the two anti-NY ESO-1 TCR (NT1 and NT1b) or one of the two 2^{nd} gen TCR-based CARs (NT2 and NT3) were prepared in the manner described above. The percentages of T cells that were NYpep/A2 positive based on APC-NYpep/A2 tetramer staining by FACS (for NT1a, NT1b, NT2, and NT3) were adjusted to 20% with untd T cells as described above.

On day 1, 1×10⁶ target cells (Saos-2) were co-cultured with 4×10⁶ of the ATC transduced with NT1a, NT1b, NT2, or NT3 in a 12-flat well tissue culture plate in a total volume of 4 ml of R10 containing 90 IU IL-2/ml for 48 hours. On day 3, the cells were transferred to a new 24-well plate. On day 6, cells from each well were split into two wells with R10 containing 180 IU IL-2/ml in a total volume of 4 ml/well. On day 10, the cells were harvested and analyzed for binding of NYpep/A2 tetramer by FACS. The results are shown in FIG. 30. As shown in the figure, live cells were gated for analysis and percentage of positive cells were shown. It was found that the percentage of NYpep/A2 positive was most significantly increased in NT2 (about 32%), while a medium increase was observed in NT3 (approx. 25%) and a decreased was observed in the NT1a and NT1b groups (about 17-19%).

### Example 8. Analysis of 2^{nd} Gen TCR-CAR and 3^{rd} Gen TCR-CAR

In this example, assays were carried out to examine and compare anti-NY-ESO-1 TCRs of a native form and TCR-CARs of 2^{nd} or 3^{rd} gen on transduced activated human T cells.

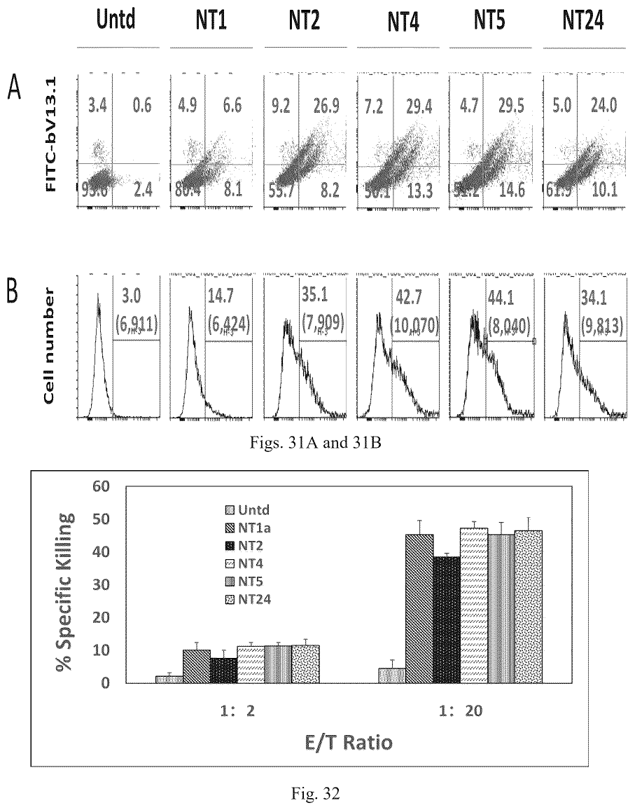

First, surface expression of the TCRs or TCR-CARs on transduced activated human T cells were examined. Briefly, anti-CD3 antibody-activated human T cells were untransduced (Untd), or transduced with virus containing supernatant derived from previously sorted-anti-NY-ESO-1 TCR+ (NT1 (also named NT1a) or anti-NY-ESO-1 TCR-CAR+ (NT2; 2^{nd} gen), NT4 (Vector 5; 2^{nd} gen), NT5 (Vector 6; 2^{nd} gen), and NT24 (Vector 11; 3^{rd} gen) cell lines as describe above. Seven to ten days post transduction, the transduced T cells were analyzed for surface expression of TCRVb13.1 and binding of NYpep/A2 tetramer by FACS. Live cells were gated for analysis. The results are shown in FIGS. 31A and B. As shown in the figures, T cells transduced with each of the three 2^{nd} gen anti-NY-ESO-1 TCR-CARs (NT2, NT4 and NT5) expressed the most and similar highest levels of TCRVb13.1 an NYpep/A2 tetramer-binding TCR or TCR-CARs as determined by TCRVb13.1 an NYpep/A2 tetramer staining by FACS. Those with the 3^{rd} gen TCR-CAR NT24 exhibited relatively lower double TCRVb13.1 and NYpep/A2 tetramer-binding+ cells, while those with the native form TCR (NT1) exhibited the lowest percentage of double TCRVb13.1 and NYpep/A2 tetramer-binding+ cells.

Second, assays were carried out to examine the ability of killing of target cells by human ATC transduced with anti-NY ESO-1 TCR or one of the four TCR-CARs post engagement with target cells. Briefly, ATC transduced with NT1 (also named NT1a), NT2, NT4, NT5, or NT24 were prepared as described above. Killing of antigen-specific target cells for these activated human T cells was carried out in vitro killing assay in which transduced human T cells (effector) were co-cultured with Saos-2 tumor cells (target) in the presence of cytokine IL-2. In the experiment, human T cells transduced individually with NT1a, NT2, NT4, NT5, or NT24 were adjusted to 15% NYpep/A+ with untd T cells, then co-cultured with Saos-2 tumor cells (target) for 12 hrs in the presence of cytokine IL-2 in the E/T ratios of 1:2 and 1:20, respectively. At the end of incubation, the T cell-mediated cytotoxicity was measured using the Cayman's 7-AAD/CFSE Cell-Mediated Cytotoxicity Assay Kit. The results are shown in FIG. 32, where the data are presented as the mean number±S.D. from three samples in each group. As shown in FIG. 32, T cells transduced with one of NT1a or one of the three TCR-based CARs (NT4, NT5 and NT24) that comprising TCR signal 2 derived from 4-1BB show the highest and similar target cell killing ability at both E/T ratios (1:2 and 1:20). Those with the NT2 (TCR-CAR of 2^{nd} gen comprising only TCR signal 2 derived from CD28) exhibited a slightly lower killing ability. No significant killing of target cells was seen with untransduced cells (Untd).

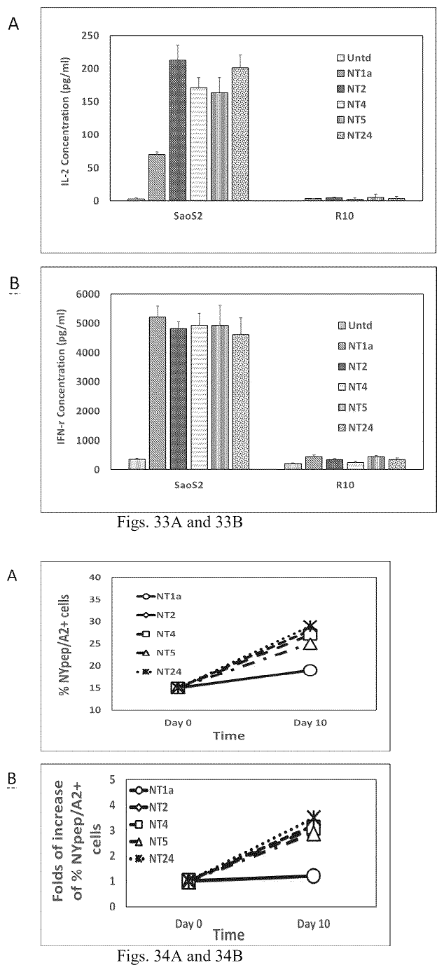

Third, cytokine secretion of human ATC transduced with the TCR or TCR-based CARs post engagement with target cells were examined. The ATC transduced with NT1a, NT2, NT4, NT5, or NT24 were prepared as described above. The transduced T cells were again adjusted to 15% NYpep/A+ with untd T cells. Then, 0.5×10⁶ target Saos-2 cells or media only (R10) were co-cultured with 2×10⁶ of the transduced ATC or untransduced human T cells (Untd) in a 24-flat well tissue culture plate in a total volume of 1.5 ml of R10 (without any IL-2 added) for 24 hours. Cytokine concentrations in the cell supernatants were measured by ELISA as described above. The results are shown in FIGS. 33A and B, where the data are presented as the mean number±S.D. from three samples in each group. As shown in FIG. 33A, those with NT2 or NT24 (both comprising one (NT24) or two (NT2) TCR signaling 2 derived from human CD28) secreted the highest amount of IL-2 (about 200 pg/ml, and 215 pg/ml, respectively). Those with the NT4 or NT5 (with TCR signal 2 from 4-1BB) produced moderate amounts (about 170 pg/ml and 165 pg/ml, respectively). Those with the native form of anti-NY-ESO-1/A2 TCR (NT1) secreted the lowest amount. For IFN-γ as shown FIG. 33B, the ATC transduced with NT1 (also named NT1a), NT2, NT4, NT5, and NT24 secreted similar levels of IFN-γ upon engagement with target Saos-2 cells, but not in the absence of target cells (R10 control). The negative control cells Untd did not produce significant amount of IL-2 or IFN-r in the presence or absence of target cells.

Finally, assays were carried out to examine changes of percentages and expansion of NYpep/A2+ ATC transduced with anti-NY ESO-1 TCR or one of the four TCR-based CARs post engagement with target cells. Briefly, on day 1, ATC transduced with NT1a, NT2, NT4, NT5 or NT24 were adjusted to 15% NYpep/A+ with untd T cells. Then, 1×10⁶ target Saos-2 cells were co-cultured with 6×10⁶ of ATC that were previously adjusted to 15% NYpep/A+ cells and consisted of 0.9×10⁶ NYpep/A+ cells in a 12-flat well tissue culture plate in a total volume of 5 ml of R10 containing 90 IU IL-2/ml for 48 hours. On day 3, all cells were transferred to a new 12-well plate. On day 6 (6 days post the initial of co-culture), cells in each well were split into two new wells with R10 containing 180 IU IL-2/ml in a total volume of 5 ml/well. On day 10, the suspended cells were harvested, counted under microscopic examination with trypan blue staining, and analyzed for binding of FITC-anti-human CD3 antibody and APC-NYpep/A2 tetramer by FACS. It was found that over 95-98% of live cells were CD3+ (data not shown). Live and CD3+ cells were gated for analysis. The results are shown in FIGS. 34A and B. The total numbers of NYpep/A2+ ATC were calculated based on formula:

total number of live cells (by trypan blue staining)×% of NYpep/A2+cells as shown in FIG. 34A/total number of NYpep/A2+ATC.

As shown in FIG. 34A, percentage of NYpep/A2 positive cells significantly increased from 15% to similar levels in all four TCR-CARs (NT2, NT4, NT5 and NT24) ranging from 25.3% to 29.2%; 28.4%, 27.1%, 25.3% and 29.2%, respectively. That for the NT1a increased slightly increased to 19.1%. As shown in FIG. 34B, the folds of increase for NT2, NT4, NT5 and NT24 were 3.2, 3.1, 2.9, and 3.5, respectively, while that for NT1a was 1.2.

### Example 9. In Vivo Anti-Tumor Activities

In this example, xenograft tumor murine models were used to examine in vivo anti-tumor activities of T-cells transduced with vectors described herein.

Briefly, 5 days prior to adoptive cell transfer of effector T cells (Day-5), Saos-2 cells were injected subcutaneously into the hind flank of SCID female mice in 100 ul PBS at e.g., 3×10⁶ cells/mouse. Activated human T cells transduced individually with retrovirus encoding NT1a, NT2, NT4, and NT24 or un-transduced human T cells (Untd) were prepared as described above for FIG. 31.

On Day 0, the mice were divided into five groups, six mice in each. ATC transduced with NT1a, NT2, NT4, NT5, or NT24 were adjusted to 15% NYpep/A+ with un-transduced T cells. The cells (containing both CD4+ and CD8+ T cells) were then administered intravenously into the tail vein at a dose of 1×10⁷ T cells/100 ul PBS/mouse.

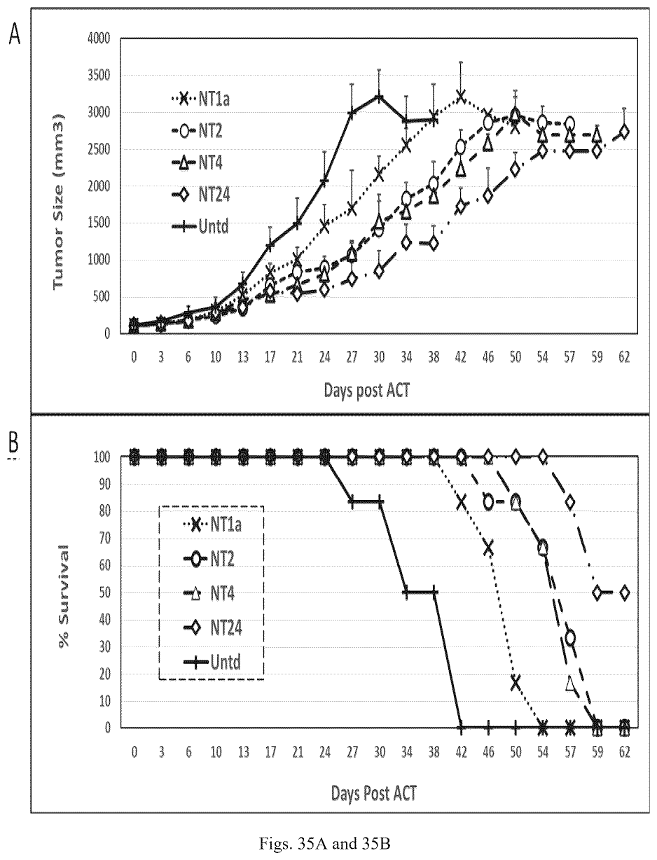

The mice were monitored for tumor growth. Tumor size was measured with calipers, and tumor volume was calculated using the formula (Tumor Measurement (mm³)=Length×Width×Height×0.5236). Mice sacrificed due to oversize of tumor or found dead were excluded from collection and analysis of data on tumor sizes. The results are shown in FIG. 35. As shown in the figures, only the NT24 group had all 6 mice (100%) still alive on day 54 post ACT administration. In contrast, 0, 4, 4, and 0 of the mice in the NT1, NT2, NT4 and Untd groups respectively were alive. In addition, at the end of the experiment on day 62 post ATC, while none of the six was alive in the NT1, NT2, NT4 and Untd group, respectively, 3 out of 6 (50%) mice in the NT24 group were alive.

These in vivo data indicate that the 3^{rd} gen anti-NY-ESO-1/A2 TCR-CAR NT24 is significantly more potent that its counter parts of 2^{nd} gen with only TCR signal 2 only from human CD28 (NT2) or only from 4-1BB (NT4), and even more potent than the native form NT1 (NT1a). The data also suggest that the both of the two 2^{nd} gen anti-NY-ESO-1 TCR-CARs show superior anti-tumor activity than the native from NT1 (NT1a).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. An antigen receptor, comprising (I) a first polypeptide chain that comprises an extracellular domain comprising a T cell receptor (TCR) beta chain or an antigen-binding fragment thereof, a transmembrane domain, and a cytoplasmic domain, and (II) a second polypeptide chain that comprises an extracellular domain comprising a TCR alpha chain or an antigen-binding fragment thereof, a transmembrane domain, and a cytoplasmic domain, wherein
the TCR beta chain and the TCR alpha chain form an antigen binding site, and
the cytoplasmic domain of the first polypeptide chain or of the second polypeptide chain comprises:
0, 1, or 2 copies of a cytoplasmic domain of a human 4-1BB or a fragment thereof, or
0, 1, or 2 copies of a cytoplasmic domain of human CD3zeta or a fragment thereof, or
0, 1, or 2 copies of a cytoplasmic domain of human CD3epsilon or a fragment thereof, or
0, 1, or 2 copies of a cytoplasmic domain of human CD28 or a fragment thereof.

2. The antigen receptor of claim 1, wherein
the first polypeptide chain comprises 1 copy of the transmembrane domain of human CD8, 1 copy of the cytoplasmic domain human CD28, and 1 copy of the cytoplasmic domain of human CD3zeta, and
the second polypeptide chain comprises 1 copy of the transmembrane domain of human CD8, 1 copy of the cytoplasmic domain human 4-1BB, and 1 copy of the cytoplasmic domain of human CD3zeta.

3. The antigen receptor of claim 1, wherein
the first polypeptide chain comprises 1 copy of the transmembrane domain of human CD8, 2 copies of the cytoplasmic domain human CD28, and 1 copy of the cytoplasmic domain of human CD3zeta, and
the second polypeptide chain comprises 1 copy of the transmembrane domain of human CD8, 2 copies of the cytoplasmic domain human 4-1BB, and 1 copy of the cytoplasmic domain of human CD3zeta.

4. The antigen receptor of claim 1, wherein the transmembrane domain of the first or the second polypeptide chain comprises one selected from the group consisting of a transmembrane domain of CD8 and a transmembrane domain of CD28.

5. An isolated nucleic acid or a set of isolated nucleic acids encoding the antigen receptor of claim 1.

6. The isolated nucleic acid or a set of isolated nucleic acids of claim 5, comprising
(I) a first nucleic acid sequence encoding the first polypeptide chain containing a first polypeptide segment of at least 10 amino acids in length and
(II) a second nucleic acid sequence encoding the second polypeptide chain containing a second polypeptide segment of at least 10 amino acids in length, wherein
the first polypeptide segment is at least 90% identical to the second polypeptide segment, and
the first nucleic acid sequence and the second nucleic acid sequence contain at least one non-identical codon within the codons that encode identical amino acid residues in the first polypeptide segment and the second polypeptide segment.

7. The isolated nucleic acid or nucleic acids of claim 6, wherein (a) the first polypeptide chain and the second polypeptide chain comprise an identical polypeptide sequence of greater than 10 amino acids in length; and (b) the first nucleic acid sequence and the second nucleic acid sequence contain at least one non-identical codon within the codons that encode the identical polypeptide sequence of the first and second nucleic acid sequences.

8. The isolated nucleic acid or nucleic acids of claim 6, wherein at least 2% of the codons are non-identical.

9. The isolated nucleic acid or nucleic acids of claim 6, wherein the first nucleic acid sequence and the second nucleic acid sequence are less than 98% identical within the codons.

10. The isolated nucleic acid or nucleic acids of claim 6, wherein the first nucleic acid sequence and the second nucleic acid sequence contain at least one non-identical codon within the codons that encode one or more selected from the group consisting of: (a) the TCR beta chain or an antigen-binding fragment thereof,
(b) the TCR alpha chain or an antigen-binding fragment thereof,
(c) the transmembrane domain of human CD8,
(d) the transmembrane domain of human CD28,
(e) the cytoplasmic domain of human CD3zeta or a fragment thereof,
(f) the cytoplasmic domain of human CD3epsilon or a fragment thereof,
(g) the cytoplasmic domain of human CD28 or a fragment thereof, and
(h) the cytoplasmic domain of human 4-1BB or a fragment thereof.

11. The isolated nucleic acid or nucleic acids of claim 10, wherein the isolated nucleic acid encode one selected from the group consisting of SEQ ID NOs: 9, 64, 10, 11, and 32-41.

12. The isolated nucleic acid or nucleic acids of claim 11, wherein the isolated nucleic acid comprises a sequence selected from the group consisting of SEQ ID NOs: 12, 65, 13, 14, and 54-63.

13. The isolated nucleic acid of claim 6, wherein the first nucleic acid sequence and the second nucleic acid sequence are linked via a nucleic acid sequence comprising an internal ribosomal entry site (IRES), an IRES DNA fragment.

14. A vector comprising the isolated nucleic acid or nucleic acids of claim 5.

15. The vector of claim 14, wherein the vector is an expression vector.

16. The vector of claim 15, wherein the vector is a viral vector.

17. A cell comprising the antigen receptor of claim 1, or a isolated nucleic acid or a set of nucleic acids encoding the antigen receptor, or a vector of comprising the nucleic acid or nucleic acids.

18. The cell of claim 17, wherein the cell is a T cell.

19. A pharmaceutical composition comprising (i) the isolated nucleic acid of claim 5, or a vector comprising the isolated nucleic acid, or a cell comprising the nucleic acid or the vector, and (ii) a pharmaceutically acceptable carrier.

20. A method of treating a tumor, or a viral infection disease, comprising administering to a subject in need thereof an effective amount of the pharmaceutical composition of claim 19.
